(19) United States
(12) Patent Application Publication
(10) Pub. No.: US 2016/0024487 A1
(43) Pub. Date: Jan. 28, 2016

(54) THROMBIN SENSITIVE COAGULATION FACTOR X MOLECULES

(71) Applicant: NOVO NORDISK A/S, Bagsværd (DK)

(72) Inventors: Jens J. Hansen, Jyllinge (DK); Jens Breinholt, Dyssegaerd (DK); Jens Buchardt, Gentofte (DK); Kristoffer Winther Balling, Roedovre (DK); Prafull S. Gandhi, Ballerup (DK); Henrik Oestergaard, Oelstykke (DK); Grant E. Blouse, Koebenhavn S (DK)

(73) Assignee: NOVO NORDISK A/S, Bagsvaerd (DK)

(21) Appl. No.: 14/774,790

(22) PCT Filed: Mar. 12, 2014
(86) PCT No.: PCT/EP2014/054841

§ 371 Date: Sep. 11, 2015

Related U.S. Application Data
(60) Provisional application No. 61/781,156, filed on Mar. 14, 2013.

(30) Foreign Application Priority Data
Mar. 12, 2013 (EP) 13158721.4

(51) Int. Cl. C12N 9/64

(52) U.S. Cl. CPC C12N 9/6432; A61K 38/00

(57) ABSTRACT

The present invention relates to thrombin sensitive coagulation Factor X (FX), as well as use thereof in medicine. In particular the invention relates to FX molecules comprising 2 to 10 amino acid modifications in the activation peptide N-terminally of the FX “IVGG” motif as well as compositions comprising such molecules and use thereof. Such molecules may be useful in connection with convenient and patient friendly treatment regimens in treatment and prophylaxis of haemophilia.

### TECHNICAL FIELD

The present invention relates to thrombin sensitive Factor X molecules as well as therapeutic and/or prophylactic use thereof.

### BACKGROUND OF THE INVENTION

Thrombin (coagulation Factor II/FIIa) is a trypsin like serine protease formed by activation of prothrombin. Thrombin is a central component of the blood coagulation cascade as its protease activity converts soluble fibrinogen into insoluble strands of fibrin, by release of Fibrinopeptide A, as well as catalysing many other coagulation-related reactions, including activation of FV, and FVIII. Thrombin cleavage sites are thus found in nature in proteins involved in coagulation.

Haemophilia is an inherited deficiency in a blood clotting factor—usually Factor VIII (FVIII)—that causes increased bleeding. Current treatment of haemophilia is based on protein replacement therapy. A particular therapeutic conundrum is the development of “inhibitors” (antibodies against coagulation factors).

Activated Factor VII (NovoSeven®) for intravenous (IV) administration has become available as a very effective “by-passing” therapy for patients with haemophilia and haemophilia with inhibitors. Factor Vila has an in vivo circulatory half-life of about 4-5 hours and it is thus desirable to provide alternative and more convenient by-passing treatment options for haemophilia patients with and without inhibitors.

Endogenous Factor X (FX) has a relatively long in vivo circulatory half-life (about 20 hours to 40 hours) and has therefore previously been suggested as a candidate for by-passing treatment of haemophilia and haemophilia with inhibitors. It is known from e.g. WO03035861 and WO2010070137 that recombinant FX variants fused with a 10 amino acid Fibrinopeptide A peptide are thrombin sensitive. Insertion of additional protease cleavage sites in FX is furthermore disclosed in US2009053185A1 and US2006148038.

Thrombin sensitivity of FX molecules will potentially result in improved and more convenient treatment options for haemophilia patients with and without inhibitors. More convenient treatment options for haemophilia patients will potentially also translate into improved compliance of prophylactic and on-demand treatments. There is thus a need in the art for further improving thrombin sensitivity of coagulation factor proteins such as FX. There is furthermore a need in the art for providing thrombin sensitive FX molecules being safe in use with regard to formation of inhibitors. There is furthermore a need in the art for thrombin sensitive FX molecules essentially without auto-activation properties. There is furthermore a need in the art for thrombin sensitive FX molecules with a long in vivo circulatory half-life and thus enabling more convenient treatments options. There is furthermore a need in the art for providing thrombin sensitive FX molecules, wherein the activated form of said molecules is essentially similar to activated wild type FX. Finally, there is a need in the art for thrombin sensitive FX molecules having low major histocompatibility complex class II (MHC II) affinity and thus low risk of inducing neutralizing anti-drug antibodies.

### SUMMARY OF THE INVENTION

The present invention relates to Factor X (FX) molecules comprising 2 to 10 amino acid modifications in the activation peptide N-terminally of the FX “IVGG” motif as well as compositions comprising such molecules and use thereof. Such compounds may be useful in connection with convenient and patient friendly treatment regimens in treatment and prophylaxis of haemophilia.

That is, the invention relates to methods of treating or preventing haemophilia, wherein said methods comprise administering a suitable dose of a thrombin sensitive Factor X molecule of the invention to a patient in need thereof.

In particular the invention provides thrombin sensitive Factor X molecules comprising 2 to 10 amino acid modifications N-terminally of the “IVGG” motif (amino acids 195 to 198 in SEQ ID NO: 1) in wild type Factor X, said modifications being in any of the positions X₁₀ to X₁ upstream of the “IVGG” motif: X₁₀, X₉, X₈, X₇, X₆, X₅, X₄, X₃, X₂, X₁, I, V, G, G wherein X₁₀ to X₁ can be any naturally occurring amino acid.

In one embodiment the thrombin sensitive Factor X molecule comprises a X₈-X₁ sequence wherein X₈ is N, X₇ is N, X₆ is A, X₅ is T, X₄ is selected from the group consisting of L, I, M, F, V, P or W, X₃ is selected from the group consisting of Q, M, R, T, W, K, I, or V, X₂ is P, and X₁ is R.

In another embodiment the thrombin sensitive Factor X molecule comprises a X₈ to X₁ sequence wherein X₈ is R, X₇ is G, X₆ is D, X₅ is N, X₄ is selected from the group consisting of L, I, M, F, V, P or W, X₃ is selected from the group consisting of T or S, X₂ is P and X₁ is R.

In another embodiment the thrombin sensitive Factor X molecule comprises a X₉ to X₁ sequence wherein X₉ is A, X₈ is T, X₇ is N, X₆ is A, X₅ is T, X₄ is selected from the group consisting of F, L, M, W, A, I, V and P, X₃ is selected from the group consisting of T, K, Q, P, S, Y, R, A, V, W, I and H, X₂ is P, and X₁ is R.

In yet another embodiment the thrombin sensitive Factor X molecule comprises a X₁₀ to X₁ sequence wherein X₁₀ is P, X₉ is E, X₈ is R, X₇ is G, X₆ is D, X₅ is N, X₄ is selected from the group consisting of L, I, M, F, V, P or W, X₃ is selected from the group consisting of T or S, X₂ is P, and X₁ is R.

In yet another embodiment the thrombin sensitive Factor X molecule comprises a X₁₀ to X₁ sequence wherein X₁₀ is P, X₉ is E, X₈ is R, X₇ is G, X₆ is D, X₅ is N, X₄ is L, X₃ is T, X₂ is P, and X₁ is R.

In yet another embodiment the thrombin sensitive Factor X molecule comprises a X₁₀ to X₁ sequence wherein X₁₀ is P, X₉ is E, X₈ is R, X₇ is G, X₆ is D, X₅ is N, X₄ is M, X₃ is T, X₂ is P, and X₁ is R.

In yet another embodiment the thrombin sensitive Factor X molecule comprises a X₁₀ to X₁ sequence wherein X₁₀ is P, X₉ is E, X₈ is R, X₇ is G, X₆ is D, X₅ is N, X₄ is M, X₃ is T, X₂ is P, and X₁ is R.

In yet another embodiment the thrombin sensitive Factor X molecule comprises a X₁₀ to X₁ sequence wherein X₁₀ is P, X₉ is E, X₈ is R, X₇ is N, X₆ is A, X₅ is T, X₄ is L, X₃ is T, X₂ is P and X₁ is R.

In yet another embodiment the thrombin sensitive Factor X molecule comprises a X₁₀ to X₁ sequence wherein X₁₀ is G, X₉ is D, X₈ is N, X₇ is N, X₆ is A, X₅ is T, X₄ is L, X₃ is T, X₂ is P and X₁ is R.

In yet another embodiment the thrombin sensitive Factor X molecule comprises a X₁₀ to X₁ sequence wherein X₁₀ is G, X₉ is G, X₈ is G, X₇ is N, X₆ is A, X₅ is T, X₄ is L, X₃ is D, X₂ is P, and X₁ is R.

In yet another embodiment the thrombin sensitive Factor X molecule comprises a X₁₀ to X₁ sequence wherein X₁₀ is S, X₉ is T, X₈ is P, X₇ is S, X₆ is I, X₅ is L, X₄ is L, X₃ is K, X₂ is P, and X₁ is R.

In yet another embodiment the thrombin sensitive Factor X molecule comprises a X₁₀ to X₁ sequence wherein X₁₀ is T, X₉ is R, X₈ is P, X₇ is S, X₆ is I, X₅ is L, X₄ is F, X₃ is T, X₂ is P, and X₁ is R.

In yet another embodiment the thrombin sensitive Factor X molecule comprises a X₁₀-X₁ sequence wherein X₁₀ is D, X₉ is F, X₈ is L, X₇ is A, X₆ is E, X₅ is G, X₄ is G, X₃ is G, X₂ is P, and X₁ is R.

In yet another embodiment the thrombin sensitive Factor X molecule comprises a X₁₀-X₁ sequence wherein X₁₀ is N, X₉ is E, X₈ is S, X₇ is T, X₆ is T, X₅ is K, X₄ is I, X₃ is K, X₂ is P, and X₁ is R.

In one embodiment the thrombin sensitive FX molecules of the invention may be protracted and have increased circulating half-life compared to a non-protracted FX molecule.

### BRIEF DESCRIPTION OF THE FIGURES

FIG. 1 shows the structure of the Factor X zymogen (including the RKR tripeptide).

FIG. 2 shows functionalization of glycyl sialic acid cytidine monophosphate (GSC) with a benzaldehyde group. GSC is acylated with 4-formylbenzoic acid and subsequently reacted with heparosan (HEP)-amine by a reductive amination reaction.

FIG. 3 shows functionalization of heparosan (HEP) polymer with a benzaldehyde group and subsequent reaction with glycyl sialic acid cytidine monophosphate (GSC) in a reductive amination reaction.

FIG. 4 shows functionalization of glycyl sialic acid cytidine monophosphate (GSC) with a thio group and subsequent reaction with a maleimide functionalized heparosan (HEP) polymer.

FIGS. 5-8 show the protein design strategies and illustrate modifications to the wild type Factor X sequence used to generate thrombin sensitive Factor X molecules.

FIG. 9 shows plasma Factor X concentrations versus time in FVIII-KO mice. The concentrations were measured by the antigen based assay after dosing the mice IV with 16.7 nmol/kg (1 mg FX/kg) of pdFX and 40 kDa HEP-[N]-pdFX. Results are mean±SD in a semi-logarithmic plot, n=3.

FIG. 10 shows a graphical representation of the final FX-AP-FpA-HPC4 construct (SEQ ID NO: 6).

### BRIEF DESCRIPTION OF THE SEQUENCE LISTING

SEQ ID NO: 1 shows the amino acid sequence of wild type mature human coagulation Factor X (zymogen).

SEQ ID NO: 2 shows the generic amino acid sequence of wild type IVGG motif and positions 2-10 upstream of the IVGG motif which may be modified.

SEQ ID NO: 3 shows the sequence of a FX-AP-FpA fusion protein disclosed in WO2010070137.

SEQ ID NO: 4 shows the nucleotide sequence used herein of a FX-AP-FpA fusion protein disclosed in WO2010070137.

SEQ ID NOs: 5-236 shows the nucleotide and amino acid sequence of thrombin sensitive mature human coagulation Factor X molecules (zymogen). Sequences are listed pairwise. For example SEQ ID NO: 5 is the nucleotide sequence encoding the polypeptide for which the amino acid sequence is listed in SEQ ID NO: 6 (FX ins[194]>[DFLAEGGGVR]-HPC4) and so forth.

SEQ ID NOs: 237 and 238 shows the sequence of a quenched fluorescence peptide substrate.

SEQ ID NO: 239 shows the open sequence of rationally designed QF-substrates.

SEQ ID NO: 240 shows a Fibrinopeptide A (FpA) substrate sequence.

SEQ ID NO: 241 shows a PAR 1 control substrate sequence.

SEQ ID NO: 242 shows a positional scanning library sequence with open positions X₄ and X₃.

SEQ ID NOs: 243-246 show the nucleotide sequence of the primers used for generating the two PCR fragments and for amplification of the fusion of the two fragments used in the cloning of FX-AP-FpA.

### DESCRIPTION

The present invention relates to thrombin sensitive FX molecules. Such molecules can e.g. be used for prophylaxis and treatment of patients suffering from haemophilia with and without inhibitors.

Thrombin is a “trypsin-like” serine protease encoded by the F2 gene in humans. Prothrombin (coagulation Factor II) is proteolytically cleaved to form thrombin in connection with the coagulation cascade. Thrombin in turn acts as a serine protease that converts soluble fibrinogen into insoluble strands of fibrin, as well as catalysing many other coagulation-related reactions.

Factor X molecules according to the present invention are “thrombin sensitive”, meaning that they can be proteolytically cleaved by thrombin. Preferably, Factor X molecules according to the present invention have thrombin sensitivity with a k_{cat}/K_{M} of at least 4.0E+02 M⁻¹ s⁻¹, preferably at least 4.0E+03 M⁻¹ s⁻¹ or 4.0E+04 M⁻¹ s⁻¹. Thrombin sensitivity of a peptide sequence and/or a coagulation factor according to the invention can be measured in e.g. chromogenic, fluorogenic, or quenched fluorescence assays (examples) generally used for measuring FXa, wherein FXa is proteolytically activated Factor X

Factor X molecules according to the present invention comprise 2 to 10 amino acid modifications which includes but is not limited to mutations/alterations/insertion(-s)/substitution(-s) and/or deletion(-s) (such as e.g. 2, 3, 4, 5, 6, 7, 8, 9, 10, 2-10, 2-9, 2-8, 2-7, 2-6, 2-5 2-4, 2-3, or 3-4 amino acid modifications) N-terminally of the IVGG motif positioned at amino acids 195-198 in the amino acid sequence as set forth in SEQ ID NO: 1. In connection with the present invention, the following numbering scheme is used for the first 10 amino acids N-terminally positioned in relation to the IVGG site (residues 185-194): X₁₀ (corresponding to Arg185 in SEQ ID NO: 1), X₉, X₈, X₇, X₆, X₅, X₄, X₃, X₂, X₁ (corresponding to Arg194 in SEQ ID NO 1), I, V, G, G (SEQ ID NO: 2). It thus follows, that 2 to 10 of the X₁₀-X₁ amino acids according to SEQ ID NO: 2 are modified relative to the corresponding sequence in the wild type Factor X sequence. In one embodiment, the amino acid modification can comprise a conservative amino acid substitution, or more than one conservative amino acid substitutions. In another embodiment, the amino acid modification can comprise a non-conservative amino acid substitution or more than one non-conservative substitution. For the purposes of clarity, the term “conservative amino acid substitution” refers to a substitution of amino acids having side chains with similar biochemical properties (e.g., non-polar and aliphatic, aromatic, hydrophobic, acidic, basic, and polar, uncharged). Conversely, a “non-conservative amino acid substitution” refers to substitution of amino acids having side chains with different biochemical properties. In another embodiment, the amino acid modifications can be in the form of an insertion of an amino acid or more than one amino acids, for example consecutive amino acids or non-consecutive amino acids. In yet another embodiment, the amino acid modifications can be in the form of a deletion of an amino acid, or a deletion of more than one amino acids, for example consecutive amino acids or non-consecutive amino acids. In yet another embodiment, the amino acid modification can comprise multiple amino acid modifications, e.g., a substitution(s), insertion(s), and/or deletion(s). For example, one or more amino acid substitutions can be combined with one or more amino acid insertions and/or deletions—in which the insertions and deletions can be consecutive or non-consecutive. The X₁₀-X₁ amino acids N-terminal of the IVGG motif thus comprise amino acids derived from the native Factor X sequence as well as amino acid substitutions, and/or deletions and/or insertions. The advantage being that FX molecules according to the invention have relatively few amino acid alterations compared to wild type Factor X and thus theoretically a safer profile in relation to e.g. risk of developing inhibitory drug antibodies. Factor X molecules according to the invention, furthermore, preferably have a relatively long in vivo circulatory half-life, enabling administration of said molecule for prophylaxis and/or treatment on a daily basis, three times a week, twice a week, once a week, once every second week, once every third week, or once monthly. FX molecules according to the invention, once activated, preferably resemble the activated form of wild type Factor X.

“MHC affinity”: The affinity of FX molecules according to the present invention towards major histocompatibility complex II molecules (MHCII affinity) can be predicted using either in silico based methods, in vitro assays or in vivo studies. In silico prediction of binding can be performed using software such as NetMHCIIpan-2.0 software (Nielsen et al. (2010) Immunome research, 6(1), 9) or NetMHCIIpan 2.1 for HLA-DR predictions (Nielsen et al., (2010) Immunome Research, 6:9) and NetMHCII 2.2 for HLA-DP/DQ predictions (Nielsen et al., (2009) BMC Bioinformatics 10:296), which estimate how binding of a given peptide sequence ranks among a large set of random peptides. In vitro assessment of binding can encompass measurements of peptide binding to recombinant MHCII molecules or using T-cell stimulation assays in which proteins or peptides are exposed to antigen presenting cells which digest the protein/peptide and present fragments of it on their MHCII molecules for recognition by the T-cell receptor; positive recognition will stimulate proliferation of the T-cell line. In vivo assessment of MHCII binding can be studied in e.g. a break of tolerance model in which animals have been tolerized to human wild type Factor X and are then exposed to thrombin sensitive Factor X variants and the development of anti Factor X variant specific antibodies monitored with respect to e.g. titers and time of occurrence.

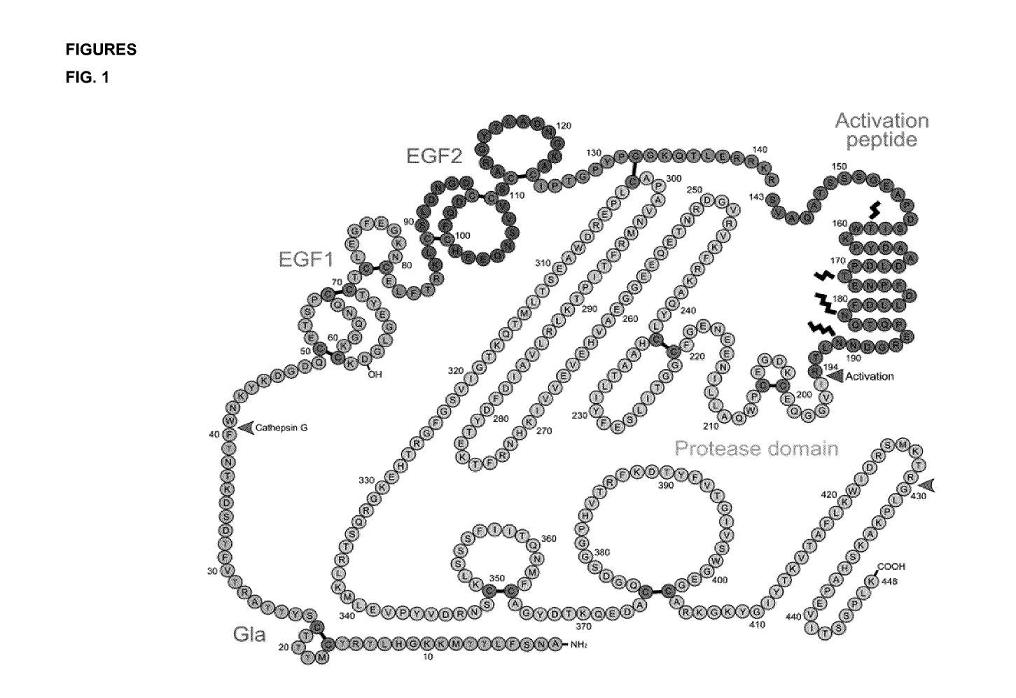

Factor X (FX) is a vitamin K-dependent coagulation factor with structural similarities to Factor VII, prothrombin, Factor IX (FIX), and protein C. It is synthesised with a 40-residue pre-pro-sequence containing a hydrophobic signal sequence (Aa1-31) that targets the protein for secretion. The pro-peptide is important for directing γ-carboxylation to the light chain of Factor X. The circulating human Factor X zymogen consists of 445 amino acids divided into four distinct domains comprising an N-terminal gamma-carboxyglutamic acid rich (Gla) domain, two EGF domains, and a C-terminal trypsin-like serine protease domain. The mature two-chain form of Factor X consists of a light chain (amino acids 41-179 (numbering according to the immature amino acid sequence)) and a heavy chain (amino acids 183-488) held together by a disulfide bridge (Cys¹⁷²-Cys³⁴² (immature amino acid sequence)) and by an excised Arg¹⁸⁰-Lys¹⁸¹-Arg¹⁸² (RKR) tripeptide found at the C-terminal end of the Factor X light chain (immature amino acid sequence). The light chain contains 11 Gla residues, which are important for Ca²⁺-dependent binding of Factor X to negatively charged phospholipid membranes. Wild type human coagulation Factor X has two N-glycosylation sites (Asn²²¹ and Asn²³¹ (immature amino acid sequence)) and two 0-glycosylation sites (Thr¹⁹⁹ and Thr²¹¹ (immature amino acid sequence)) in the activation peptide (AP). It has previously been shown that the N-glycans in the activation peptide appear to be mainly responsible for the relatively long half-life of endogenous Factor X. β-hydroxylation occurs at Asp¹⁰³ in the first EGF domain (immature amino acid sequence), resulting in 6-hydroxyaspartic acid (Hya). FIG. 1 is a structural depiction of the FX zymogen (including the RKR tripeptide) with numbering according to the mature Factor X polypeptide.

Activation of Factor X occurs by limited thrombin proteolysis at Arg²³⁴-Ile²³⁵ releasing a 52 amino acid activation peptide (amino acids 183-234). To resemble wild type Factor X following activation, Factor X molecules according to the present invention preferably comprise the wild type Factor X prime site sequence of IVGG (Ile²³⁵, Val²³⁶, Gly²³⁷, Gly²³⁸—corresponding to amino acids 195-198 according to SEQ ID NO: 1) at the activation cleavage site. Factor X molecules according to the present invention comprise 2 to 10 alterations/modifications in the X₁₀-X₁ amino acid residues according to SEQ ID NO: 2 that result in increased thrombin sensitivity. In assays that measure the rates of thrombin cleavage of quenched fluorescence thrombin substrates with identical X₄-X₁ residues (and prime-site IVGG), but having varied X₈-X₅ amino acids have similar k_{cat}/K_{M} values (see example 3). Preferably, an N-linked glycan corresponding to Asn²³¹ (numbering according to the immature molecule) is retained in the present position (or optionally at a different position if insertions and/or deletions have been introduced).

Administration of thrombin sensitive Factor X molecules according to the present invention is thought to be able to “boost” thrombin generation/production, thereby having the potential to “by-pass” e.g. FVIII and/or FIX deficiency. Molecules according to the present invention are thus being suitable for treatment of haemophilia A or B, with and without inhibitors as well as Factor X deficiency. Use of Factor X molecules according to the present invention is thought to enable convenient and patient friendly regiments where administration can take place e.g. twice a week, once a week, once every second week, once every third week, once a month or once every second month.

“Factor X deficiency” is a rare autosomal recessive bleeding disorder with an incidence of 1:1,000,000 in the general population (Dewerchin et al. (2000) Thromb Haemost 83: 185-190). Although it produces a variable bleeding tendency, patients with a severe Factor X deficiency tend to be the most seriously affected among patients with rare coagulation defects. The prevalence of heterozygous Factor X deficiency is about 1:500, but is usually clinically asymptomatic.

One example of a “wild type Factor X” is the full length mature human FX molecule, as shown in SEQ ID NO: 1.

“Factor X” or “FX” herein refers to any functional Factor X protein molecule capable of activating prothrombin, including functional fragments, analogues and derivatives of SEQ ID NO: 1. “Factor X molecules” or “FX molecules” is used broadly and comprise both wild type FX and the thrombin sensitive FX derivatives according to the present invention.

Factor X molecules according to the present invention preferably have wild type Factor X activity in the activated form. In one embodiment, Factor X molecules according to the invention are at least 90% identical (preferably at least 91%, 92%, 93%, 94%, 95%, 96%, 97%, 98%, or 99% identical) with wild type Factor X—the zymogen amino acid sequence thereof is as set forth in SEQ ID NO: 1. Preferably, activated Factor X molecules according to the invention are identical to wild type activated Factor X, in which case all amino acid modifications are placed e.g. within the activation peptide.

Factor X according to the invention is a recombinant protein produced using well known methods of production and purification. The degree and location of glycosylation, γ-carboxylation and other post-translational modifications may vary depending on the chosen host cell and its growth conditions.

### Further Description of the Sequences

SEQ ID NO: 1 gives the amino acid sequence of wild type mature human coagulation Factor X (zymogen). Activation peptide marked with bold—light chain marked with lower case letters and heavy chain marked with underline, positions corresponding to the X₁₀-X₁ amino acids are marked with bold and underline, and the IVGG motif are shown with enlarged capital bold and underlined letters:

ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQ [symbol]
GGTILSEFYIL
TAAHCLYQAKRFKVRVGDRNTEQEEGGEAVHEVEVVIKHNRFTKETYDF
DIAVLRLKTPITFRMNVAPACLPERDWAESTLMTQKTGIVSGFGRTHEK
GRQSTRLKMLEVPYVDRNSCKLSSSFIITQNMFCAGYDTKQEDACQGDS
GGPHVTRFKDTYFVTGIVSWGEGCARKGKYGIYTKVTAFLKWIDRSMKT
RGLPKAKSHAPEVITSSPLK

SEQ ID NO: 2 gives the amino acid sequence framework for Factor X molecules according to the present invention which comprises the IVGG motif from the wild type molecule and from 2 to 10 amino acid modifications in the region upstream of the IVGG motif: X₁₀, X₉, X₈, X₇, X₆, X₅, X₄, X₃, X₂, X₁, I, V, G, G

SEQ ID NO: 3 gives the amino acid sequence of an FX-FpA fusion protein disclosed in WO2010070137. Activation peptide is shown in bold, the inserted FpA sequence is shown in italics and heavy chain shown in underline.

ansfleemkkghlerecmeetcsyeearevfedsdktnefwnkykdgd
qcetspcqnqgkckdglgeytctclegfegkncelftrklcsldngdc
dqfcheeqnsvvcscargytladngkaciptgpypcgkqtlerrkrSV
AQATSSSGEAPDSITWKPYDAADLDPTENPFDLLDFNQTQPERGDNNL
TRDFLAEGGGVRIVGGQECKDGECPWQALLINEENEGFCGGTILSEFY
ILTAAHCLYQAKRFKVRVGDRNTEQEEGGEAVHEVEVVIKHNRFTKET
YDFDIAVLRLKTPITFRMNVAPACLPERDWAESTLMTQKTGIVSGFGR
THEKGRQSTRLKMLEVPYVDRNSCKLSSSFIITQNMFCAGYDTKQEDA
CQGDSGGPHVTRFKDTYFVTGIVSWGEGCARKGKYGIYTKVTAFLKWI
DRSMKTRGLPKAKSHAPEVITSSPLK

SEQ ID NOs: 5-236 give the amino acid sequences for thrombin sensitive human coagulation Factor X molecules (zymogen). For the selected exemplar mature thrombin sensitive human coagulation Factor X molecules listed below, the activation peptide is shown in bold; light chain marked with lower case letters and heavy chain are shown in underline, positions corresponding to the X₁₀-X₁ amino acids are shown in bold and underline, amino acid modifications (modification/mutations/alterations) are shown in bold, underline and italics and the IVGG motif is shown in enlarged CAPITAL, bold and underlined letters:

SEQ ID NO: 16
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETY
DFDIAVLRLKTPITFRMNVAPACLPERDWAESTLMTQKTGIVSGFGRT
HEKGRQSTRLKMLEVPYVDRNSCKLSSSFIITQNMFCAGYDTKQEDAC
QGDSGGPHVTRFKDTYFVTGIVSWGEGCARKGKYGIYTKVTAFLKWID
RSMKTRGLPKAKSHAPEVITSSPLK
SEQ ID NO: 20
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 24
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 28
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 32
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 36
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
GFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 40
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
GFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 48
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
GFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO 52:
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 56
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 64
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 72
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
EGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 76
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQPER [symbol]
GFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 108
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQ [symbol]
ENEGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 112
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQ [symbol]
ENEGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK
SEQ ID NO: 116
ansfleemkkghlerecmeetcsyeearevfedsdktnefwnkykdgdq
cetspcqnqgkckdglgeytctclegfegkncelftrklcsldngdcdq
fcheeqnsvvcscargytladngkaciptgpypcgkqtlerrkrSVAQA
TSSSGEAPDSITWKPYDAADLDPTENPFDLLDFNQTQ [symbol]
ENEGFCGGTILSEFYI
LTAAHCLYQAKRFKVRVGDRNTEQEEGGEAVHEVEVVIKHNRFTKETYD
FDIAVLRLKTPITFRMNVAPACLPERDWAESTLMTQKTGIVSGFGRTHE
KGRQSTRLKMLEVPYVDRNSCKLSSSFIITQNMFCAGYDTKQEDACQGD
SGGPHVTRFKDTYFVTGIVSWGEGCARKGKYGIYTKVTAFLKWIDRSMK
TRGLPKAKSHAPEVITSSPLK

The term “haemophilia”/“coagulopathy”/“blood clotting disease”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic.

Non-limiting examples of congenital hypocoagulopathies are haemophilia A, haemophilia B, Factor VII deficiency, Factor X deficiency, Factor XI deficiency, von Willebrand's disease and thrombocytopenias such as Glanzmann's thrombasthenia and Bernard-Soulier syndrome. Said haemophilia A or B may be severe, moderate or mild. The clinical severity of haemophilia is determined by the concentration of functional units of FIX/FVIII in the blood and is classified as mild, moderate, or severe. Severe haemophilia is defined by a clotting factor level of <0.01 U/ml corresponding to <1% of the normal level, while moderate and mild patients have levels from 1-5% and >5%, respectively. Haemophilia A with “inhibitors” (that is, allo-antibodies against Factor VIII) and haemophilia B with “inhibitors” (that is, allo-antibodies against Factor IX) are non-limiting examples of coagulopathies that are partly congenital and partly acquired.

In one embodiment of the current invention, haemorrhage is associated with haemophilia A or B. In another embodiment, haemorrhage is associated with haemophilia A or B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intra-cranially, intra-aurally or intraocularly. In another embodiment, haemorrhage is associated with anticoagulant therapy.

The term “treatment”, as used herein, refers to the medical therapy of any human or other vertebrate subject in need thereof. Said treatment may be prophylactic and/or therapeutic.

“Mode of administration”: Compounds according to the invention may be administered parenterally, e.g. intravenously, intramuscularly, subcutaneously, or intradermally. Compounds according to the invention may be administered prophylactically and/or therapeutically (on demand).

Compounds according to the invention may be co-administered with one or more other therapeutic agents or formulations. The other agent may be an agent that enhances the effects of the compounds of the invention. The other agent may be intended to treat other symptoms or conditions of the patient. For example, the other agent may be an analgesic, other types of coagulation factors or compounds modulating haemostasis and/or fibrinolysis.

The compounds according to the invention may be produced by means of recombinant nucleic acid techniques. In general, a DNA sequence encoding a molecule according to the invention is inserted into an expression vector, which is in turn transformed or transfected (transiently or stably) into host cells. The host cell (e.g. a yeast cell, an insect cell or a mammalian cell) is subsequently incubated under conditions suitable for expressing the molecule. The Factor X molecule can subsequently be isolated.

The invention also relates to polynucleotides that encode Factor X molecules of the invention. Thus, a polynucleotide of the invention may encode any Factor X molecule as described herein. The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

A polynucleotide of the invention may encode a polypeptide comprising the sequence of inter alia SEQ ID NOs: 3, 8, 108, 112, 120, 160 or a variant or fragment thereof. Such a polynucleotide may consist of or comprise a nucleic acid sequence of any one of SEQ ID NOs: 4, 7, 107, 111, 119 or 159. A suitable polynucleotide sequence may alternatively be a variant of one of these specific polynucleotide sequences. For example, a variant may be a substitution, deletion or addition variant of any of the above nucleic acid sequences.

In another aspect, the present invention provides pharmaceutical compositions/formulations comprising Factor X molecules according to the invention. For example, the invention provides pharmaceutical compositions formulated together with one or more pharmaceutically acceptable carrier (e.g. the use of preservatives, isotonic agents, chelating agents, stabilizers and surfactants in pharmaceutical compositions is well-known to the skilled person). Preferably, the pharmaceutical formulation is a freeze-dried formulation, to which the physician or the patient adds solvents and/or diluents prior to use. In a further aspect, the pharmaceutical formulation comprises an aqueous solution and a buffer, wherein the coagulation factor is present in a concentration from 1 mg/ml or above, and wherein said formulation has a pH from about 6.0 to about 8.0, such as e.g. about 6.0, 6.1, 6.2, 6.3, 6.3, 6.4, 6.5, 6.5, 6.6, 6.7, 6.8, 6.7, 6.8, 6.9, 7.0, 7.1, 7.2, 7.3, 7.4, 7.5, 7.6, 7.7, 7.8, 7.8, 7, 9, or 8.0.

“FX derivative” according to the present invention, is intended to designate Factor X molecules according to the invention exhibiting substantially the same or improved biological activity relative to wild type Factor X, in which one or more of the amino acids have been chemically modified, e.g. by alkylation, PEGylation, acylation, ester formation or amide formation or the like.

The term “protractive groups”/“half-life extending moieties” is herein understood to refer to one or more chemical groups attached to one or more Factor X amino acid side chain functionalities such as —SH, —OH, —COOH, —CONH₂, —NH₂, or one or more N- and/or O-glycan structures. Said half-life extending moieties can increase in vivo circulatory half-life of a number of therapeutic proteins/peptides when conjugated to these proteins/peptides. Examples of protractive groups/half-life extending moieties include: Biocompatible fatty acids and derivatives thereof, polysaccarides (e.g. Hydroxy Alkyl Starch (HAS) e.g. Hydroxy Ethyl Starch (HES), Hyaluronic acid (HA), Dextran, Poly-sialic acids (PSA) and Heparosan polymers (HEP)), Poly Ethylene Glycol (PEG), Poly (Glyₓ-Ser_{y})ₙ (HAP), Phosphorylcholine-based polymers (PC polymer), Fleximers, polypeptides (e.g. Fc domains, Transferrin, Albumin, Elastin like peptides, XTEN polymers, Albumin binding peptides, and CTP peptides), and any combination thereof.

“PEGylated coagulation factors” according to the present invention may have one or more polyethylene glycol (PEG) molecules attached to any part of the protein, including any amino acid residue or carbohydrate moiety. Chemical and/or enzymatic methods can be employed for conjugating PEG (or other half-life extending moieties) to a glycan on the protein according to the invention. An example of an enzymatic conjugation process is described e.g. in WO03031464, which is hereby incorporated by reference in its entirety.

The glycan may be naturally occurring or it may be inserted via e.g. insertion of an N-linked glycan using recombinant methods well known in the art. According to a preferred embodiment, Factor X molecules/derivatives according to the invention are conjugated with half-life extending moieties at one or more of the glycans present in the activation peptide, in which case said half-life extending moieties are removed upon activation of the molecule.

“HEPylated coagulation factors” according to the present invention may a heparosan (HEP) polymer attached to any part of the protein, including any amino acid residue or carbohydrate moiety.

“Cysteine-conjugated (e.g. acylated/pegylated, etc.) coagulation factors molecules/derivatives” according to the present invention have one or more half-life extending moieties conjugated to a sulfhydryl group of a cysteine that is present or is introduced in the protein. It is, furthermore, possible to link protractive half-life extending moieties to other amino acid residues.

“Cysteine-PEGylated coagulation factors” according to the present invention have one or more PEG molecules conjugated to a sulfhydryl group of a cysteine present or introduced in the protein.

“Cysteine-HEPylated coagulation factors” according to the present invention have one or more HEP molecules conjugated to a sulfhydryl group of a cysteine present or introduced in the protein.

“Heparosan” (HEP) is a natural sugar polymer comprising (-GlcUA-1,4-GlcNAc-1,4-) repeats. It belongs to the glycosaminoglycan polysaccharide family and is a negatively charged polymer at physiological pH. It can be found in the capsule of certain bacteria but it is also found in higher vertebrate where it serves as precursor for the natural polymers heparin and heparan sulphate. HEP can be degraded by lysosomal enzymes such as N-acetyl-a-D-glucosaminidase (NAGLU) and β-glucuronidase (GUSB). A heparosan polymer for use in the present invention is typically a polymer of the formula (-GlcUA-beta1,4-GlcNAc-alpha1,4-)ₙ. The size of the HEP polymer may be defined by the number of repeats n. The number of said repeats n may be, for example, from 2 to about 5,000. The number of repeats may be, for example 50 to 2,000 units, 100 to 1,000 units, 5 to 450 or 200 to 700 units. The number of repeats may be 200 to 250 units, 500 to 550 units or 350 to 400 units. Any of the lower limits of these ranges may be combined with any higher upper limit of these ranges to form a suitable range of numbers of units in the HEP polymer.

The size of the HEP polymer may also be defined by its molecular weight. The molecular weight may be the average molecular weight for a population of HEP polymer molecules, such as the weight average molecular mass. Molecular weight values as described herein in relation to size of the HEP polymer may not, in practise, exactly be the size listed. Due to batch to batch variation during HEP polymer production, some variation is to be expected. To encompass batch to batch variation, it is therefore to be understood, that a variation around +/−10%, 9%, 8%, 7%, 6%, 5%, 4%, 3%, 2% or 1% around target HEP polymer size could to be expected. For example, a HEP polymer size of 40 kDa denotes 40 kDa+/−10%, e.g. 40 kDa could for example in practise mean 38.8 kDa or 41.5 kDa.

The HEP polymer may have a molecular weight of, for example, 500 Da to 1,000 kDa. The molecular weight of the polymer may be 500 Da to 650 kDa, 5 to 750 kDa, 10 to 500 kDa, 15 to 550 kDa, 25 to 250 kDa or 50 to 175 kDa.

For the purposes of the present invention the molecular weight may be selected at particular levels within these ranges in order to achieve a suitable balance between activity of the Factor X molecule and half-life of the conjugate. For example, the molecular weight of the HEP polymer may be in a range selected from 5 to 15 kDa, 15 to 25 kDa, 25 to 35 kDa, 35 to 45 kDa, 45 to 55 kDa, 55 to 65 kDa, 65 to 75 kDa, 75 to 85 kDa, 85 to 95 kDa, 95 to 105 kDa, 105 to 115 kDa, 115 to 125 kDa, 125 to 135 kDa, 135 to 145 kDa, 145 to 155 kDa, 155 to 165 kDa or 165 to 175 kDa. In other embodiments, the molecular weight may be 500 Da to 21 kDa, such as 1 kDa to 15 kDa, such as 5 to 15 kDa, such as 8 to 17 kDa, such as 10 to 14 kDa such as about 12 kDa. The molecular weight may be 20 to 35 kDa, such as 22 to 32 kDa such as 25 to 30 kDa, such as about 27 kDa. The molecular weight may be 35 to 65 kDa, such as 40 to 60 kDa, such as 47 to 57 kDa, such as 50 to 55 kDa such as about 52 kDa. The molecular weight may be 50 to 75 kDa such as 60 to 70 kDa, such as 63 to 67 kDa such as about 65 kDa. The molecular weight may be 75 to 125 kDa, such as 90 to 120 kDa, such as 95 to 115 kDa, such as 100 to 112 kDa, such as 106 to 110 kDa such as about 108 kDa. The molecular weight may be 125 to 175 kDa, such as 140 to 165 kDa, such as 150 to 165 kDa, such as 155 to 160 kDa such as about 157 kDa. The molecular weight may be 5 to 100 kDa, such as 13 to 60 kDa and such as 27 to 40 kDa.

In particularly interesting embodiments, the HEP polymer conjugated to the FX molecule has a size in a range selected from 13 to 65 kDa, 13 to 55 kDa, 13 to 50 kDa, 13 to 49 kDa, 13 to 48 kDa, 13 to 47 kDa, 13 to 46 kDa, 13 to 45 kDa, 13 to 44 kDa, 13 to 43 kDa, 13 to 42 kDa, 13 to 41 kDa, 13 to 40 kDa, 13 to 39 kDa, 13 to 38 kDa, 13 to 37 kDa, 13 to 36 kDa, 13 to 35 kDa, 13 to 34 kDa, 13 to 33 kDa, 13 to 33 kDa, 13 to 32 kDa, 13 to 31 kDa, 13 to 30 kDa, 13 to 29 kDa, 13 to 28 kDa, 13 to 27 kDa, 13 to 26 kDa, 13 to 25 kDa, 13 to 21 kDa, 25 to 55 kDa, 25 to 50 kDa, 25 to 45 kDa, 27 to 40 kDa, 27 to 41 kDa, 27 to 42 kDa, 27 to 43 kDa, 27 to 43 kDa, 27 to 44 kDa, 30 to 45 kDa and 38 to 42 kDa.

Any of the lower limits of these ranges of molecular weight may be combined with any higher upper limit from these ranges to form a suitable range for the molecular weight of the HEP polymer in accordance with the invention.

In connection with FX conjugates as described herein, use of HEP in the side chain offers a very flexible way of prolonging in vivo circulation half-life since a range of HEP sizes result in a significantly improved half-life.

The HEP polymer may have a narrow size distribution (i.e. monodisperse) or a broad size distribution (i.e. polydisperse). The level of polydispersity may be represented numerically based on the formula Mw/Mn, where Mw=weight average molecular mass and Mn=number average molecular weight. The polydispersity value using this equation for an ideal monodisperse polymer is 1. Preferably, a HEP polymer for use in the present invention is monodisperse. The polymer may therefore have a polydispersity that is about 1, the polydispersity may be less than 1.25, preferably less than 1.20, preferably less than 1.15, preferably less than 1.10, preferably less than 1.09, preferably less than 1.08, preferably less than 1.07, preferably less than 1.06, preferably less than 1.05. The molecular weight size distribution of the HEP may be measured by comparison with monodisperse size standards (HA Lo-Ladder, Hyalose LLC) which may be run on agarose gels.

Alternatively, the size distribution of HEP polymers may be determined by high performance size exclusion chromatography-multi angle laser light scattering (SEC-MALLS). Such a method can be used to assess the molecular weight and polydispersity of a HEP polymer. Polymer size may be regulated in enzymatic methods of production. By controlling the molar ratio of HEP acceptor chains to UDP sugar, it is possible to select a final HEP polymer size that is desired.

HEP polymers can be prepared by a synchronised enzymatic polymerisation reaction (US 20100036001). This method use heparan synthetase I from Pasturella multocida (PmHS1) which can be expressed in E. coli as a maltose binding protein fusion constructs. Purified MBP-PmHS1 is able to produce monodisperse polymers in a synchronized, stoichiometrically controlled reaction, when it is added to an equimolar mixture of sugar nucleotides (GlcNAc-UDP and GlcUA-UDP). A trisaccharide initiator (GlcUA-GlcNAc-GlcUA) is used to prime the reaction, and polymer length is determined by the primer:sugar nucleotide ratios. The polymerization reaction will run until about 90% of the sugar nucleotides are consumed. Polymers are isolated from the reaction mixture by anion exchange chromatography, and subsequently freeze-dried into stable powder.

According to the present invention, a Factor X molecule as described herein is conjugated to a HEP polymer as described herein. Any Factor X molecule as described herein may be combined with any HEP polymer as described herein. Common methods for linking half-life extending moieties such as carbohydrate polymers to glycoproteins comprise oxime, hydrazone or hydrazide bond formation. WO2006094810 describes methods for attaching hydroxyethyl starch polymers to glycoproteins such as erythropoietin that circumvent the problems connected to using activated ester chemistry. In these methods, hydroxyethyl starch and erythropoietin are individually oxidized with periodate on the carbohydrate moieties, and the reactive carbonyl groups ligated together using bis-hydroxylamine linking agents. The method will create hydroxyethyl starch linked to the erythropoietin via oxime bonds. Similar oxime based linking methodology can be imagined for attaching carbohydrate polymers to GSC (cf. WO2011101267), however, as such oxime bonds are known to exist in both syn- and anti-isomer forms, the linkage between the polymer and the protein will contain both syn- and anti-isomer combinations. Such isomer mixtures are usually not desirable in proteinaceous medicaments that are used for long term repeating administration since the linker inhomogeneity may pose a risk for antibody generation.

The above mentioned methods have further disadvantages. In the oxidative process required for activating the glycoprotein, parts of the carbohydrate residues are chemically cleaved and the carbohydrates will therefore not present in intact form in the final conjugate. The oxidative process will, furthermore, generate product heterogenicity as the oxidating agent i.e. periodate in most cases is unspecific with regard to which glycan residue is oxidized. Both product heterogenecity and the presence of non-intact glycan residues in the final drug conjugate may impose immunogenicity risks.

Alternatives for linking carbohydrate polymers to glycoproteins involve the use of maleimide chemistry (WO2006094810). For example, the carbohydrate polymer can be furnished with a maleimido group, which selectively can react with a sulfhydryl group on the target protein. The linkage will then contain a cyclic succinimide group.

In connection with the present invention, it is shown that it is possible to link a carbohydrate polymer such as HEP via a maleimido group to a thio-modified GSC molecule and transfer the reagent to an intact glycosyl groups on a glycoprotein by means of a sialyltransferase, thereby creating a linkage that contains a cyclic succinimide group. Succinimide based linkages, however, may undergo hydrolytic ring opening when the conjugate is stored in aqueous solution for extended time periods (Bioconjugation Techniques, G. T. Hermanson, Academic Press, 3^{rd} edition 2013 p. 309) and while the linkage may remain intact, the ring opening reaction will add undesirable heterogeneity in form of regio- and stereo-isomers to the final conjugate.

It follows from the above that it is preferable to link the half-life extending moiety to the glycoprotein in such a way that 1) the glycan residue of the glycoprotein is preserved in intact form, and 2) no heterogeneity is present in the linker part between the intact glycosyl residue and the half-life extending moiety.

There is a need in the art for methods of conjugating a half-life extending moiety such as HEP to a protein glycan such as a Factor X glycan, wherein the compounds are linked such that a stable and isomer free conjugate is obtained.

In one aspect the present invention provides a stable and isomer free linker for use in glycyl sialic acid cytidine monophosphate (GSC) based conjugation of HEP to Factor X. The GSC starting material used in the current invention can be synthesised chemically (Dufner, G. Eur. J. Org. Chem. 2000, 1467-1482) or it can be obtained by chemoenzymatic routes as described in WO07056191. The GSC structure is shown below:

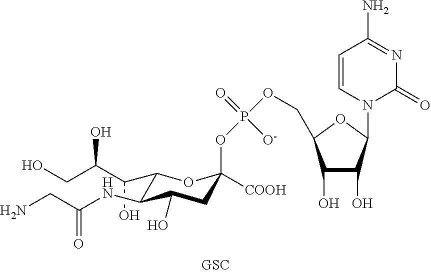

In one embodiment conjugates according to the present invention comprise a linker comprising the following structure:

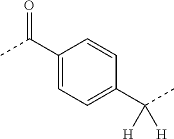

hereinafter also referred to as sublinker or sublinkage—that connects a HEP-amine and GSC in one of the following ways:

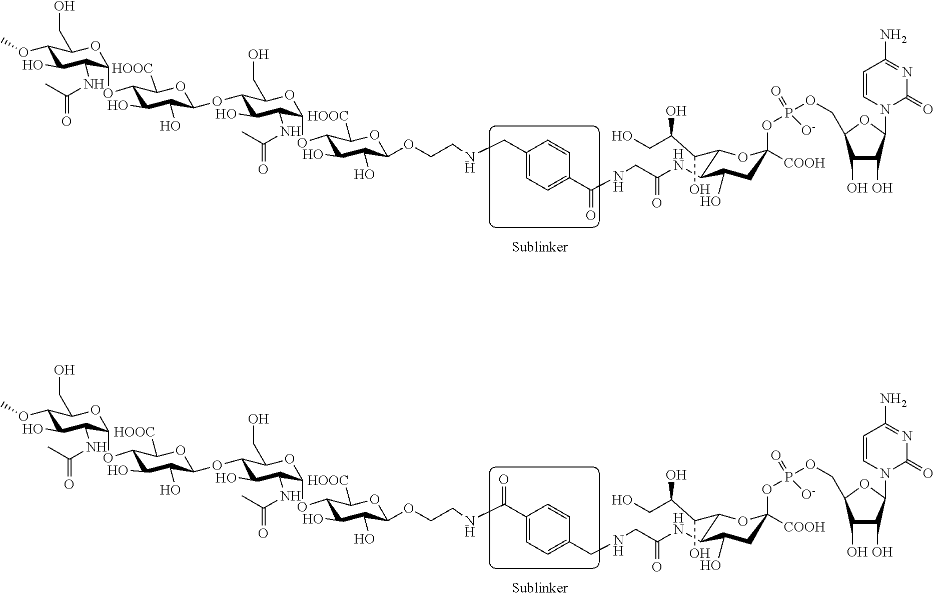

The highlighted 4-methylbenzoyl sublinker thus makes up part of the full linking structure linking the half-life extending moiety to a target protein. The sublinker is as such a stable structure compared to alternatives, such as succinimide based linkers (prepared from maleimide reactions with sulfhydryl groups) since the latter type of cyclic linkage has a tendency to undergo hydrolytic ring opening when the conjugate is stored in aqueous solution for extended time periods (Bioconjugation Techniques, G. T. Hermanson, Academic Press, 3^{rd} edition 2013 p. 309). Even though the linkage in this case (e.g. between HEP and sialic acid on a glycoprotein) may remain intact, the ring opening reaction will add heterogeneity in form of regio- and stereo-isomers to the final conjugate composition. One advantage associated with conjugates according to the present invention is thus that a homogenous composition is obtained, i.e. that the tendency of isomer formation due to linker structure and stability is significantly reduced. Another advantage is that the linker and conjugates according to the invention can be produced in a simple process, preferably a one-step process.

Isomers are undesirable since these can lead to a heterogeneous product and increase the risk for unwanted immune responses in humans. The 4-methylbenzoyl sublinkage as used in the present invention between HEP and GSC is not able to form steno- or regio isomers.

Processes for preparation of functional HEP polymers are described in US 20100036001 that for example lists aldehyde-, amine- and maleimide functionalized HEP reagents. US 20100036001 is hereby incorporated by reference in its entirety as if fully set forth herein. A range of other functionally modified HEP derivatives are available using similar chemistry. HEP polymers used in certain embodiments of the present invention are initially produced with a primary amine handle at the reducing terminal according to methods described in US20100036001.

Amine functionalized HEP polymers (i.e. HEP having an amine-handle) prepared according US20100036001 can be converted into a HEP-benzaldehyde by reaction with N-succinimidyl 4-formylbenzoate and subsequently coupled to the glycylamino group of GSC by a reductive amination reaction. The resulting HEP-GSC product can subsequently be enzymatically conjugated to a glycoprotein using a sialyltransferase.

For example said amine handle on HEP can be converted into a benzaldehyde functionality by reaction with N-succinimidyl 4-formylbenzoate according to the below scheme:

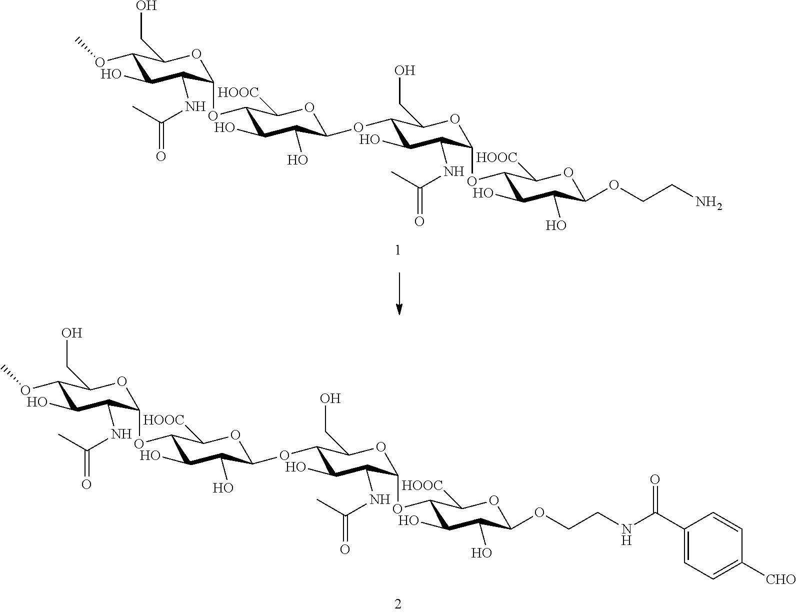

The conversion of HEP amine (1) to the 4-formylbenzamide compound (2) in the above scheme may be carried out by reaction with acyl activated forms of 4-formylbenzoic acid.

N-succinimidyl may be chosen as acyl activating group but a number of other acyl activation groups are known to the skilled person. Non-limited examples include 1-hydroxy-7-azabenzotriazole-, 1-hydroxy-benzotriazole-, pentafluorophenyl-esters as know from peptide chemistry.

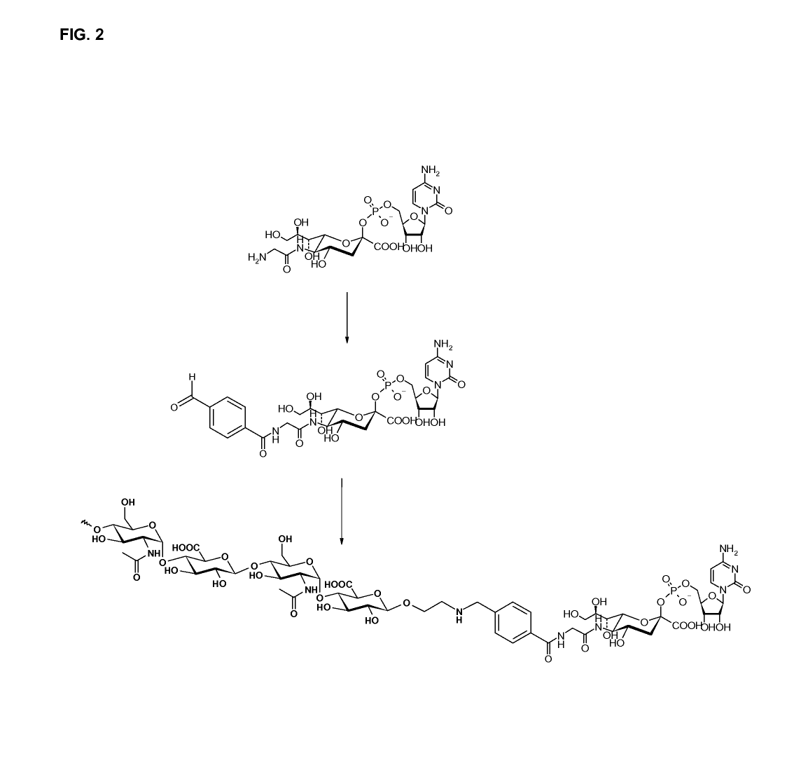

HEP reagents modified with a benzaldehyde functionality can be kept stable for extended time periods when stored frozen (−80° C.) in dry form. Alternatively, a benzaldehyde moiety can be attached to the GSC compound, thereby resulting in a GSC-benzaldehyde compound suitable for conjugation to an amine functionalized HEP moiety. This route of synthesis is depicted in FIG. 2.

For example, GSC can be reacted under pH neutral conditions with N-succinimidyl 4-formylbenzoate to provide a GSC compound that contains a reactive aldehyde group. The aldehyde derivatized GSC compound (GSC-benzaldehyde) can then be reacted with HEP-amine and reducing agent to form a HEP-GSC reagent.

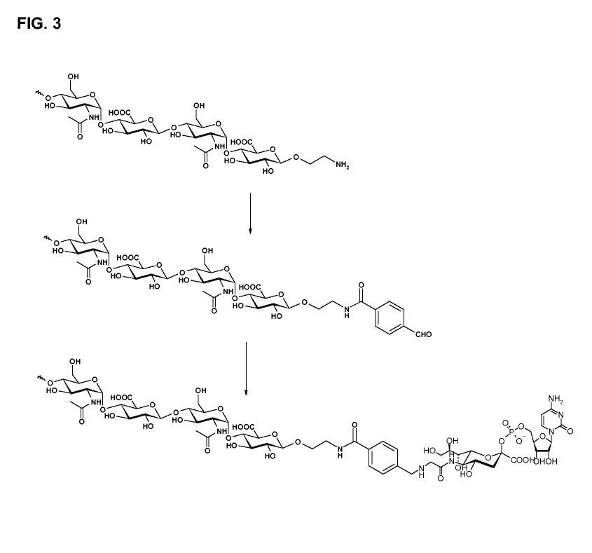

The above mentioned reaction may be reversed, so that the HEP-amine is first reacted with N-succinimidyl 4-formylbenzoate to form an aldehyde derivatized HEP-polymer, which subsequently is reacted directly with GSC in the presence of a reducing agent. In practice this eliminates the tedious chromatographic handling of GSC-CHO. This route of synthesis is depicted in FIG. 3. Thus, in one embodiment of the present invention HEP-benzaldehyde is coupled to GSC by reductive amination.

Reductive amination is a two-step reaction which proceeds as follows: Initially an imine (also known as Schiff-base) is formed between the aldehyde component and the amine component (in the present embodiment the glycyl amino group of GSC). The imine is then reduced to an amine in the second step. The reducing agent is chosen so that it selectively reduces the formed imine to an amine derivative.

A number of suitable reducing reagents are available to the skilled person. Non-limiting examples include sodium cyanoborohydride (NaBH3CN), sodium borohydride (NaBH4), pyridin boran complex (BH3:Py), dimethylsulfide boran complex (Me2S:BH3) and picoline boran complex.

Although reductive amination to the reducing end of carbohydrates (for example to the reducing termini of HEP polymers) is possible, it has generally been described as a slow and inefficient reaction (J C. Gildersleeve, Bioconjug Chem. 2008 July; 19(7): 1485-1490). Side reactions, such as the Amadori reaction, where the initially formed imine rearrange to a keto amine are also possible, and will lead to heterogeneity which as previously discussed is undesirable in the present context.

Aromatic aldehydes such as benzaldehydes derivatives are not able to form such rearrangement reactions as the imine is unable to enolize and also lack the required neighbouring hydroxy group typically found in carbohydrate derived imines. Aromatic aldehydes such as benzaldehydes derivatives are therefore particular useful in reductive amination reactions for generating the isomer free HEP-GSC reagent.

A surplus of GSC and reducing reagent is optionally used in order to drive reductive amination chemistry fast to completion. When the reaction is completed, the excess (non-reacted) GSC reagent and other small molecular components such as excess reducing reagent can subsequently be removed by dialysis, tangential flow filtration or size exclusion chromatography.

Both the natural substrate for sialyltransferases, Sia-CMP, and the GSC derivatives are multifunctional molecules that are charged and highly hydrophilic. In addition, they are not stable in solution for extended time periods especially if pH is below 6.0. At such low pH, the CMP activation group necessary for substrate transfer is lost due to acid catalyzed phosphate diester hydrolysis. Selective modification and isolation of GSC and Sia-CMP derivatives thus require careful control of pH, as well as fast and efficient isolation methods, in order to avoid CMP-hydrolysis.

In one aspect of the present invention, large half-life extending moieties are conjugated to GSC using reductive amination chemistry. Arylaldehydes, such as benzaldehyde modified HEP polymers have been found optimal for this type of modification, as they can efficiently react with GSC under reductive amination conditions.

As GSC may undergo hydrolysis in acid media, it is important to maintain a near neutral or slightly basic environment during the coupling to HEP-benzaldehyde. HEP polymers and GSC are both highly water soluble and aqueous buffer systems are therefore preferable for maintaining pH at a near neutral level. A number of both organic and inorganic buffers may be used; however, the buffer components should preferably not be reactive under reductive amination conditions. This excludes for instance organic buffer systems containing primary and—to lesser extend—secondary amino groups. The skilled person will know which buffers are suitable and which are not. Some examples of suitable buffers are include Bicine (N,N-bis(2-hydroxyethyl)glycine), HEPES (4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (Piperazine-N,N′-bis(2-ethanesulfonic acid)) and MES (2-(N-morpholino)ethanesulfonic acid).

By applying this method, GSC reagents modified with half-life extending moieties such as HEP, having isomer free stable linkages can efficient be prepared, and isolated in a simple process that minimize the chance for hydrolysis of the CMP activation group. By reacting either of said compounds with each other a HEP-GSC conjugate comprising a 4-methylbenzoyl sublinker moiety may be created.

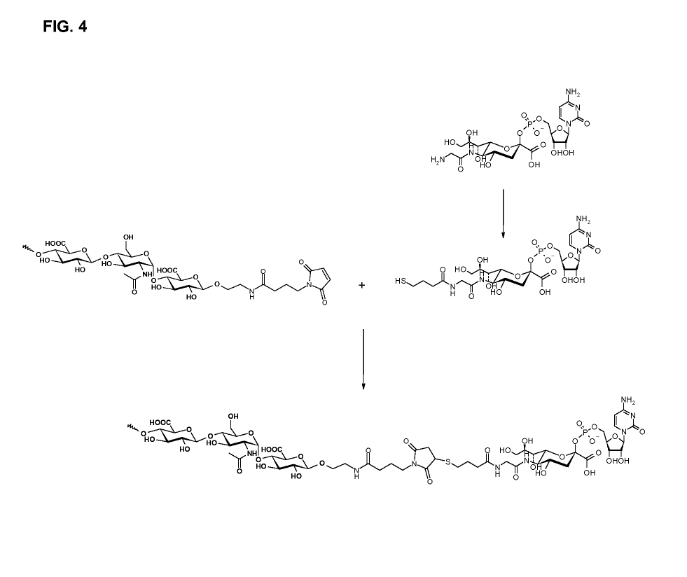

GSC may also be reacted with thiobutyrolactone, thereby creating a thiol modified GSC molecule (GSC-SH). Such reagents may be reacted with maleimide functionalized HEP polymers to form HEP-GSC reagents. This synthesis route is depicted in FIG. 4. The resulting product has a linkage structure comprising succinimide.

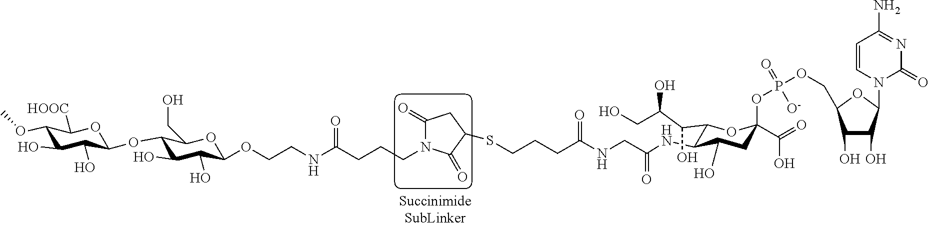

However, succinimide based (sub)linkages may undergo hydrolytic ring opening inter alia when the modified GSC reagent is stored in aqueous solution for extended time periods and while the linkage may remain intact, the ring opening reaction will add undesirable heterogeneity in form of regio- and stereo-isomers.

### Methods of Glycoconjugation

Conjugation of a HEP-GSC conjugate with a polypeptide may be carried out via a glycan present on residues in the polypeptide backbone. This form of conjugation is also referred to as glycoconjugation.

In contrast to conjugation methods based on cysteine alkylations, lysine acylations and similar conjugations involving amino acids in the protein backbone, conjugation via glycans is an appealing way of attaching larger structures such as a HEP polymer to bioactive proteins with less disturbance of bioactivity. This is because glycans being highly hydrophilic generally tend to be oriented away from the protein surface and out in solution, leaving the binding surfaces that are important for the proteins activity free. The glycan may be naturally occurring or it may be inserted via e.g. insertion of an N-linked glycan using methods well known in the art.

Methods for glycoconjugation of HEP polymers include galactose oxidase based conjugation (WO2005014035) and periodate based conjugation (WO08025856). Methods based on sialyltransferase have over the years proven to be mild and highly selective for modifying N-glycans or O-glcyans on blood coagulation factors, such as Factor X.

GSC is a sialic acid derivative that can be transferred to glycoproteins by the use of sialyltransferases. It can be selectively modified with substituents such as PEG or HEP on the glycyl amino group and still be enzymatically transferred to glycoproteins by use of sialyltransferases. GSC can be efficiently prepared by an enzymatic process in large scale (WO07056191).

In one aspect of the present invention, terminal sialic acids on Factor X glycans can be removed by sialidase treatment to provide asialoFX. AsialoFX and GSC modified with HEP together will act as substrates for sialyltransferases. The product of the sialyltransferase reaction is a HEP-FX conjugate having HEP linked via an intact glycosyl linking group on the glycan.

### Sialyltransferases

Sialyltransferases are a class of glycosyltransferases that transfer sialic acid from naturally activated sialic acid (Sia)-CMP (cytidine monophosphate) compounds to galactosyl-moieties on e.g. proteins. Many sialyltransferases (ST3GaIIII, ST3GaII, ST6GaINAcI) are capable of transfer of sialic acid-CMP (Sia-CMP) derivatives that have been modified on the C5 acetamido group inter alia with large groups such as 40 kDa PEG (WO03031464). An extensive, but non-limited list of relevant sialyltransferases that can be used with the current invention is disclosed in WO2006094810, which is hereby incorporated by reference in its entirety.

In one aspect of the present invention, terminal sialic acids on glycoproteins can be removed by sialidase treatment to provide asialo glycoproteins. Asialo glycoproteins and GSC modified with the half-life extending moiety together will act as substrates for sialyltransferases. The product of the reaction is a glycoprotein conjugate having the half-life extending moiety linked via an intact glycosyl linking group—in this case an intact sialic acid linker group.

### Properties of HEP-FX Conjugates

A conjugate of the invention may show various advantageous biological properties. For example, the conjugate may show one of more of the following non-limiting advantages when compared to a suitable control Factor X molecule: improved circulation half-life in vivo, improved mean residence time in vivo and improved biodegradability in vivo.

Advantages may be seen when a conjugate of the invention is compared to a suitable control Factor X molecule. The control molecule may be, for example, an unconjugated Factor X molecule. The conjugated control may be a Factor X molecule conjugated to a water soluble polymer, or a Factor X molecule chemically linked to a protein. A conjugated Factor X control may be a Factor X polypeptide that is conjugated to a chemical moiety (being protein or water soluble polymer) of a similar size as the HEP molecule in the conjugate of interest. The water-soluble polymer can for example be PEG, branched PEG, dextran, poly(l-hydroxymethylethylene hydroxymethylformal) or 2-methacryloyloxy-2′-ethyltrimethylammoniumphosphate (MPC).

The Factor X molecule in the control Factor X molecule is preferably the same Factor X molecule that is present in the conjugate of interest. For example, the control Factor X molecule may have the same amino acid sequence as the Factor X polypeptide in the conjugate of interest. The control Factor X may have the same glycosylation pattern as the Factor X polypeptide in the conjugate of interest.

The presently disclosed conjugates preferably show an improvement in circulatory half-life, or in mean residence time when compared to a suitable control. Conjugates according to the present invention have a modified circulatory half-life compared to the wild type protein molecule, preferably an increased circulatory half-life. Circulatory half-life is preferably increased at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, more preferably at least 125%, more preferably at least 150%, more preferably at least 175%, more preferably at least 200%, and most preferably at least 250% or 300%. Even more preferably, such molecules have a circulatory half-life that is increased at least 400%, 500%, 600%, or even 700%.

Where the activity being compared is a biological activity of Factor X, such as clotting activity or proteolysis, the control can be a suitable Factor X molecule conjugated to a water soluble polymer of comparable size to the HEP conjugate of the current invention.

The conjugate may not retain the level of biological activity seen in Factor X that is not modified by the addition of HEP. Preferably the conjugate of the invention retains as much of the biological activity of unconjugated Factor X as possible. For example, the conjugate may retain at least 15%, at least 20%, at least 25%, at least 30%, at least 35%, at least 40%, at least 45%, at least 50%, at least 60%, at least 70%, at least 80% or at least 90% of the biological activity of an unconjugated Factor X control. As discussed above, the control may be a Factor X molecule having the same amino acid sequence as the Factor X molecule in the conjugate, but lacking HEP. The conjugate may, however, show an improvement in biological activity when compared to a suitable control. The biological activity here may be any biological activity of Factor X as described herein such as clotting activity or proteolysis activity.

An advantage of the conjugates of the invention is that HEP polymers are enzymatically biodegradable. A conjugate of the invention is therefore preferably enzymatically degradable in vivo.

The term “sialic acid” refers to any member of a family of nine-carbon carboxylated sugars. The most common member of the sialic acid family is N-acetylneuraminic acid (2-keto-5-acetamido-3,5-dideoxy-D-glycero-D-galactononulopyranos-1-onic acid (often abbreviated as Neu5Ac, NeuAc, NeuNAc, or NANA). A second member of the family is N-glycolyl-neuraminic acid (Neu5Gc or NeuGc), in which the N-acetyl group of NeuNAc is hydroxylated. A third sialic acid family member is 2-keto-3-deoxy-nonulosonic acid (KDN) (Nadano et al. (1986) J. Biol. Chem. 261: 11550-11557; Kanamori et al., (1990) J. Biol. Chem. 265: 21811-21819). Also included are 9-substituted sialic acids such as a 9-O-C1-C6 acyl-Neu5Ac like 9-O-lactylNeu5Ac or 9-O-acetyl-Neu5Ac. The synthesis and use of sialic acid compounds in a sialylation procedure is disclosed in international application WO92/16640, published Oct. 1, 1992.

The term “sialic acid derivative” refers to sialic acids as defined above that are modified with one or more chemical moieties. The modifying group may for example be alkyl groups such as methyl groups, azido- and fluoro groups, or functional groups such as amino or thiol groups that can function as handles for attaching other chemical moieties. Examples include 9-deoxy-9-fluoro-Neu5Ac and 9-azido-9-deoxy-Neu5Ac. The term also encompasses sialic acids that lack one of more functional groups such as the carboxyl group or one or more of the hydroxyl groups. Derivatives where the carboxyl group is replaced with a carboxamide group or an ester group are also encompassed by the term. The term also refers to sialic acids where one or more hydroxyl groups have been oxidized to carbonyl groups. Furthermore the term refers to sialic acids that lack the C9 carbon atom or both the C9-C8 carbon chain for example after oxidative treatment with periodate.

Glycyl sialic acid is a sialic acid derivative according to the definition above, where the N-acetyl group of NeuNAc is replaced with a glycyl group also known as an amino acetyl group. Glycyl sialic acid may be represented with the following structure:

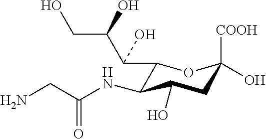

The term “CMP-activated” sialic acid or sialic acid derivatives refer to a sugar nucleotide containing a sialic acid moiety and a cytidine monophosphate (CMP). In the present description, the term “glycyl sialic acid cytidine monophosphate” is used for describing GSC, and is a synonym for alternative naming of same CMP activated glycyl sialic acid. Alternative naming include CMP-5′-glycyl sialic acid, cytidine-5′-monophospho-N-glycylneuraminic acid, cytidine-5′-monophospho-N-glycyl sialic acid. The term “intact glycosyl linking group” refers to a linking group that is derived from a glycosyl moiety in which the saccharide monomer interposed between and covalently attached to the polypeptide and the HEP moiety is not degraded, e.g., oxidized, e.g., by sodium metaperiodate during conjugate formation. “Intact glycosyl linking groups” may be derived from a naturally occurring oligosaccharide by addition of glycosyl unites or removal of one or more glycosyl unit from a parent saccharide structure.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

Open-ended dotted lines in structure formulas denotes open valence bond (i.e. bonds that connect the structures to other chemical moieties).

“Fusion proteins” according to the invention are proteins created through the in-frame joining of two or more DNA sequences which originally encode separate proteins or peptides or fragments hereof. Translation of the DNA sequence encoding a fusion protein will result in a protein sequence which may have functional properties derived from each of the original proteins or peptides. DNA sequences encoding fusion proteins may be created artificially by standard molecular biology methods such as overlapping PCR or DNA ligation and the assembly is performed excluding the stop codon in the first 5′-end DNA sequence while retaining the stop codon in the 3′end DNA sequence. The resulting fusion protein DNA sequence may be inserted into an appropriate expression vector that supports the heterologous fusion protein expression in host organisms such as e.g. bacteria, yeast, fungus, insect cells or mammalian cells.

Fusion proteins may contain a linker or spacer peptide sequence that separates the protein or peptide parts of the fusion protein. The linker or spacer peptide sequence may facilitate the correct folding of the individual protein or peptide parts and may make it more likely for the individual protein or peptide parts to retain their individual functional properties. Linker or spacer peptide sequences may be inserted into fusion protein DNA sequences during the in frame assembly of the individual DNA fragments that make up the complete fusion protein DNA sequence i.e. during overlapping PCR or DNA ligation.

The term “Fc fusion protein” is herein meant to encompass coagulation factors according to the invention fused to an Fc domain that can be derived from any antibody isotype. An IgG Fc domain will often be preferred due to the relatively long circulatory half-life of IgG antibodies. The Fc domain may furthermore be modified in order to modulate certain effector functions such as e.g. complement binding and/or binding to certain Fc receptors. Fusion with an Fc domain, which has the capacity to bind to FcRn receptors, will generally result in a prolonged circulatory half-life of the fusion protein compared to the half-life of the wild type coagulation factor. Mutations in amino acid positions 234, 235 and 237 in an IgG Fc domain will generally result in reduced binding to the FcγRI receptor and possibly also the FcγRIIa and the FcγRIII receptors. These mutations do not alter binding to the FcRn receptor, which promotes a long circulatory half-life by an endocytic recycling pathway. Preferably, a modified IgG Fc domain of a fusion protein according to the invention comprises one or more of the following mutations that will result in decreased affinity to certain Fc receptors (L234A, L235E, and G237A) and in reduced C1q-mediated complement fixation (A330S and P331S), respectively. Alternatively, the Fc domain may be an IgG4 Fc domain, preferably comprising the S241P/S228P mutation.

### The Following are Non-Limiting Aspects of the Invention

1. A thrombin sensitive Factor X molecule comprising 2 to 10 amino acid modifications N-terminally of the “IVGG” motif (amino acids 195 to 198 in SEQ ID NO: 1) in wild type Factor X, said modifications being in any of the positions X₁₀ to X₁:
   X₁₀, X₉, X₈, X₇, X₆, X₅, X₄, X₃, X₂, X₁, I, V, G, G
   wherein X₁₀ to X₁ can be any naturally occurring amino acid.
2. The thrombin sensitive Factor X molecule according to aspect 1, wherein
   X₈ is N
   X₇ is N
   X₆ is A
   X₅ is T
   X₄ is selected from the group consisting of L, I, M, F, V, P or W
   X₃ is selected from the group consisting of Q, M, R, T, W, K, I, or V
   X₂ is P, and
   X₁ is R.
3. The thrombin sensitive Factor X molecule according to aspect 1, wherein
   X₈ is R
   X₇ is G
   X₆ is D
   X₅ is N
   X₄ is selected from the group consisting of L, I, M, F, V, P or W
   X₃ is selected from the group consisting of T or S
   X₂ is P, and
   X₁ is R.
4. The thrombin sensitive Factor X molecule according to aspect 3, wherein X₄ is selected from the list consisting of: F, L, M and W.
5. The thrombin sensitive Factor X molecule according to aspect 3, wherein X₃ is T and X₄ is F.
6. The thrombin sensitive Factor X molecule according to aspect 3, wherein X₃ is T and X₄ is M.
7. The thrombin sensitive Factor X molecule according to aspect 3, wherein X₃ is T and X₄ is W.
8. The thrombin sensitive Factor X molecule according to aspect 3, wherein X₃ is T and X₄ is L.
9. The thrombin sensitive Factor X molecule according to aspect 1, wherein
   X₉ is A
   X₈ is T
   X₇ is N
   X₆ is A
   X₅ is T
   X₄ is selected from the group consisting of F, L, M, W, A, I, V and P
   X₃ is selected from the group consisting of T, K, Q, P, S, Y, R, A, V, W, I and H
   X₂ is P, and
   X₁ is R.
10. The thrombin sensitive Factor X molecule according to aspect 9, wherein X₃ is selected from the list consisting of: T, K and Q.
11. The thrombin sensitive Factor X molecule according to aspect 9, wherein X₄ is selected from the list consisting of: F, L and M.
12. The thrombin sensitive Factor X molecule according to aspect 9, wherein X₃ is T and X₄ is F.
13. The thrombin sensitive Factor X molecule according to aspect 9, wherein X₃ is T and X₄ is M.
14. The thrombin sensitive Factor X molecule according to aspect 9, wherein X₃ is T and X₄ is W.
15. The thrombin sensitive Factor X molecule according to aspect 9, wherein X₃ is T and X₄ is L.
16. The thrombin sensitive Factor X molecule according to aspect 9, wherein X₃ is K and X₄ is L.
17. The thrombin sensitive Factor X molecule according to aspect 9, wherein X₃ is K and X₄ is F.
18. The thrombin sensitive Factor X molecule according to aspect 9, wherein X₃ is K and X₄ is M.
19. The thrombin sensitive Factor X molecule according to aspect 9, wherein X₃ is Q and X₄ is W.
20. The thrombin sensitive Factor X molecule according to aspect 9, wherein X₃ is P and X₄ is W.
21. The thrombin sensitive Factor X molecule according to aspect 1, wherein
   X₁₀ is P
   X₉ is E
   X₈ is R
   X₇ is G
   X₆ is D
   X₅ is N
   X₄ is selected from the group consisting of L, I, M, F, V, P or W
   X₃ is selected from the group consisting of T or S
   X₂ is P, and
   X₁ is R.
22. The thrombin sensitive Factor X molecule according to aspect 1, wherein
   X₁₀ is P
   X₉ is E
   X₈ is R
   X₇ is G
   X₆ is D
   X₅ is N
   X₄ is L
   X₃ is T
   X₂ is P, and
   X₁ is R.
23. The thrombin sensitive Factor X molecule according to aspect 1, wherein
   X₁₀ is P
   X₉ is E
   X₈ is R
   X₇ is N
   X₆ is A
   X₅ is T
   X₄ is L
   X₃ is T
   X₂ is P, and
   X₁ is R.
24. The thrombin sensitive Factor X molecule according to aspect 1, wherein
   X₁₀ is G
   X₉ is G
   X₈ is G
   X₇ is N
   X₆ is A
   X₅ is T
   X₄ is L
   X₃ is D
   X₂ is P, and
   X₁ is R.
25. The thrombin sensitive Factor X molecule according to aspect 1, wherein
   X₁₀ is S
   X₉ is T
   X₈ is P
   X₇ is S
   X₆ is I
   X₅ is L
   X₄ is L
   X₃ is K
   X₂ is P, and
   X₁ is R.
26. The thrombin sensitive Factor X molecule according to aspect 1, wherein
   X₁₀ is S
   X₉ is T
   X₈ is P
   X₇ is S
   X₆ is 1
   X₅ is L
   X₄ is F
   X₃ is K
   X₂ is P, and
   X₁ is R.
27. The thrombin sensitive Factor X molecule according to aspect 1, wherein
   X₁₀ is T
   X₉ is R
   X₈ is P
   X₇ is S
   X₆ is I
   X₅ is L
   X₄ is F
   X₃ is T
   X₂ is P, and
   X₁ is R.
28. The thrombin sensitive Factor X molecule according to aspect 1, wherein
   X₁₀ is D
   X₉ is F
   X₈ is L
   X₇ is A
   X₆ is E
   X₅ is G
   X₄ is G
   X₃ is G
   X₂ is P, and
   X₁ is R.
29. The thrombin sensitive Factor X molecule according to aspect 1, wherein
   X₁₀ is N
   X₉ is E
   X₈ is S
   X₇ is T
   X₆ is T
   X₅ is K
   X₄ is I
   X₃ is K
   X₂ is P, and
   X₁ is R.
30. The thrombin sensitive Factor X molecule according to any one of the previous aspects, wherein the amino acid sequence of the Factor X molecule differs from the sequence of wild type Factor X by insertion, deletion, and/or substitution of one or more amino acids in Factor X regions outside X₁₀ to X₁.
31. A pharmaceutical formulation comprising the Factor X molecule according to any one of aspects 1 to 30 and optionally one or more pharmaceutically acceptable excipients.
32. The thrombin sensitive Factor X molecule according to any one of aspects 1 to 30 for use in treatment of haemophilia.
33. The thrombin sensitive Factor X molecule according to aspect 1, wherein the Ile in the IVGG motif (amino acid 195 in SEQ ID NO: 1) is selected from the list consisting of: I, L, T and V.
34. A method of treating haemophilia in a patient in need thereof comprising administering the thrombin sensitive Factor X molecule according any one of aspects 1 to 30.
35. A method of preparing the thrombin sensitive Factor X molecule according to any one of aspects 1 to 30.
36. The thrombin sensitive Factor X molecule according to any one of aspects 1 to 30, wherein said Factor X molecule is covalently conjugated to a half-life extending moiety via a glycan in the activation peptide.
37. The thrombin sensitive Factor X molecule according to any one of aspects 1 to 30, wherein said Factor X molecule is covalently conjugated to a half-life extending moiety via a cysteine residue in the activation peptide.
38. A FX molecule according to any one of aspects 1 to 30 for use in treatment of Factor X deficiency.
39. A DNA sequence encoding a recombinant Factor X molecule according to any one of aspects 1 to 30.
40. An expression vector comprising the DNA sequence according to any one of aspects 1 to 30.
41. A host cell comprising an expression vector according to aspect 40 or a DNA sequence according to aspect 39.
42. A method of producing the thrombin sensitive Factor X molecule according to any one of aspects 1 to 30, wherein said method comprises incubating a host cell according to the invention under suitable conditions and subsequently isolating said Factor X molecule.

The following list of further embodiments is not to be understood in any limiting sense. All embodiments can be combined.

A Factor X molecule comprising 2 to 10 amino acid modifications (such as 2, 3, 4, 5, 6, 7, 8, 9 or 10 amino acid modifications) in the activation peptide (N-terminally of the FX “IVGG” motif). IVGG motif positions: amino acids 195-198 in SEQ ID NO: 1.

A Factor X molecule according to the invention, comprising the following amino acid sequence: X₁₀, X₉, X₈, X₇, X₆, X₅, X₄, X₃, X₂, X₁ I, V, G, G (SEQ ID NO: 2), wherein X₁, X₂, X₃, X₄, X₅, X₆, X₇, and X₈ can be any naturally occurring amino acid. The list of naturally occurring amino acids include: G, A, V, L, I, S, T, C, M, P, D, N, E, Q, K, R, H, F, Y, and W.

A Factor X molecule according to the invention, wherein said Factor X molecule comprises 2-4 amino acid substitutions, such as 2, 3, or 4 amino acid substitutions.

A Factor X molecule according to the invention, wherein no modifications are made to X₈-X₅. Thus: X₈ is R, X₇ is G, X₆ is D, and X₅ is N. (X₄, X₃, X₂, and X₁ can be any naturally occurring amino acid) wherein the preferred X₁ is R, the preferred X₂ is P, the preferred X₃ is selected from Q, M, R, T, W, K, I, or V and the preferred X₄ is selected from L, I, M, F, V, P or W.

A Factor X molecule according to the invention, wherein no modifications are made to X₁₀-X₅ and X₂-X₁. Thus: said FX molecule preferably comprises two amino acid substitutions and X₁₀ is P, X₉ is E, X₈ is R, X₇ is G X₆ is D, X₅ is N, X₂ is T, X₁ is R (wherein X₃ and X₄ can be any naturally occurring amino acid, except L at X₃ and N at X₄).

A Factor X molecule according to the invention, wherein said molecule comprises proline at position X₂.

A Factor X molecule according to the invention, wherein X₄ is substituted with a hydrophobic or aliphatic amino acid, preferably selected from the list consisting of: L, M, I, F, V, P, and W and X₃ is not a negatively charged amino acid, preferably selected from the list consisting of: Q, M, R, T, W, K, I, and V.

A Factor X molecule according to the invention, wherein X₄ is selected from the list consisting of: L, M, I, F, V, P, W.

A Factor X molecule according to the invention, wherein no modifications are made to X₁₀, X₉, X₈, X₇, and X₆ and X₃, X₂, and X₁. Thus, said FX molecule preferably comprises two amino acid substitutions, wherein X₅ and X₄ can be any naturally occurring amino acid, except N at X₅ and N at X₄.

A Factor X molecule according to the invention, wherein X₂ and X₃ can be any naturally occurring amino acid, except T at position X₂ and L at position X₃.

A Factor X molecule according to the invention, wherein X₃ and X₄ can be any naturally occurring amino acid, except L at position X₃ and N at position X₄.

A Factor X molecule according to the invention, wherein no modifications are made to X₁₀, X₉, X₈, X₇, X₆, X₅, X₄, and X₃. Thus said Factor X molecule preferably comprises two amino acid substitutions, wherein X₂ and X₁ can be any naturally occurring amino acid, except T at X₂ and R at X₁. Preferably, X₁ is R. Preferably, X₂ is P.

A Factor X molecule according to the invention, wherein the Ile in the IVGG motif (amino acid 195 in SEQ ID NO. 1) is substituted with L, T or V.

A Factor X molecule according to the invention, wherein X₁ is preferably R.

A Factor X molecule according to the invention, wherein X₂ is preferably P.

A Factor X molecule according to the invention, wherein said molecule comprises no amino acid insertions.

A Factor X molecule according to the invention, wherein X₃ is T or S, X₂ is P, and X₁ is R.

A Factor X molecule according to the invention, wherein said molecule comprises two amino acid substitutions in the activation peptide

A Factor X molecule according to the invention, wherein said molecule comprises three amino acid substitutions in the activation peptide.

A Factor X molecule according to the invention, wherein said molecule comprises four amino acid substitutions in the activation peptide.

A Factor X molecule according to the invention, wherein said molecule comprises an N glycosylation sequence motif (N, X, T/S) in the X₁-X₁₀ motif N-terminally of the IVGG site.

A Factor X molecule according to the invention, wherein said molecule comprises at least one additional glycosylation site. Preferably, said at least one additional glycosylation site is inserted in the activation peptide and is preferably an N-glycosylation site.

A Factor X molecule according to the invention, wherein X₈ is N, X₇ is N, X₆ is A, X₅ is T, X₄ is selected from L, I, M, F, V, P or W, X₃ is selected from Q, M, R, T, W, K, I, or V, X₂ is P and X₁ is R.

A Factor X molecule according to the invention, wherein where X₈ is R, X₇ is G, X₆ is D, X₅ is N, X₄ is selected from L, I, F, M or W, X₃ is T or S, X₂ is P and X₁ is R.

A Factor X molecule according to the invention, wherein said molecule is conjugated with a half-life extending moiety.

A Factor X molecule according to the invention, wherein said half-life extending moiety is a polysaccharide such as e.g. PSA or HEP.

A Factor X molecule according to any one of the preceding embodiments, wherein said half-life extending moiety is selected from the list consisting of: Biocompatible fatty acids and derivatives thereof, Hydroxy Alkyl Starch (HAS) e.g. Hydroxy Ethyl Starch (HES), Poly Ethylene Glycol (PEG), Poly (Glyₓ-Ser_{y})ₙ (HAP), Hyaluronic acid (HA), Heparosan polymers (HEP), Phosphorylcholine-based polymers (PC polymer), Fleximers, Dextran, Poly-sialic acids (PSA), an Fc domain, Transferrin, Albumin, Elastin like peptides, XTEN polymers, Albumin binding peptides, and CTP peptides.

A Factor X molecule according to the invention, wherein said half-life extending moiety is covalently conjugated to FX via a glycan in the activation peptide.

A Factor X molecule according to the invention, wherein said half-life extending moiety is covalently conjugated to FX via a sialic acid.

A Factor X molecule according to the invention, wherein essentially no auto-activation of said molecule occurs. This can be measured in e.g. a buffered solution or in a plasma sample (e.g. as disclosed in the examples).

A Factor X molecule according to the invention, wherein said molecule has increased activity and/or rate of activation (e.g. as disclosed in the examples).

A Factor X molecule according to the invention, wherein the in silico predicted MHC II affinity of the altered sequence and flanking 15 amino acids on both sides of the insertion, deletion, and/or substitution in said coagulation factor ranks lower than the top 3% of a large set of random peptides. Preferably, the affinity is lower than the altered region and flanking 15 amino acids in SEQ ID NO: 3.

A Factor X molecule according to the invention, wherein the in vitro MHC II affinity in a cell-free system is lower than the MHC II affinity of wild type Factor X.

A Factor X molecule according to the invention, wherein the in vivo MHC II affinity is lower than the MHC II affinity of wild type Factor X.

A Factor X molecule according to the invention, wherein said molecules does not stimulate T cell proliferation in a cell based assay.

A Factor X molecule according to the invention, wherein activation of said molecule results in removal of X₈-X₁.

A Factor X molecule according to the invention, wherein activation of said molecule results in removal of X₁₀-X₁.

A Factor X molecule according to the invention, wherein X₄-X₁ comprises at least two amino acids substitutions.

A pharmaceutical formulation comprising a Factor X molecule according to the invention and optionally one or more pharmaceutically acceptable excipients.

A liquid aqueous formulation comprising a Factor X molecule according to the invention and one or more excipients, wherein one or more of said excipients have inhibitory effects on Factor X activity.

A Factor X molecule according to the invention, or a pharmaceutical formulation according to the invention for use in treatment of haemophilia.

A Factor X molecule according to the invention, or a pharmaceutical formulation according to the invention for use in treatment of haemophilia with inhibitors.

A Factor X molecule according to the invention, or a pharmaceutical formulation according to the invention for use in treatment of blood loss in connection with surgery and/or trauma.

A Factor X molecule according to the invention, or a pharmaceutical formulation according to the invention for use in treatment of Factor X deficiency.

A DNA sequence encoding a recombinant coagulation factor according to the invention.

An expression vector comprising the DNA sequence according to the invention.

A host cell comprising an expression vector according to the invention or a DNA sequence according to the invention.

A method of producing a Factor X molecule according to the invention, wherein said method comprises incubating a host cell according to the invention under suitable conditions and subsequently isolating said Factor X molecule.

A pharmaceutical composition according to the invention, wherein said composition is for IV administration.

A pharmaceutical composition according to the invention, wherein said composition is for subcutaneous or intradermal administration.

A method of making a pharmaceutical composition according to the invention, wherein said method comprises mixing a Factor X molecule according to the invention with one or more pharmaceutically acceptable excipients.

A method of treating haemophilia in a subject, wherein said method comprises administering a therapeutic amount of a Factor X molecule according to the invention, or a pharmaceutical composition according to the invention.

A method of treating haemophilia with inhibitors in a subject, wherein said method comprises administering a therapeutic amount of a Factor X molecule according to the invention, or a pharmaceutical composition according to the invention.

### EXAMPLES

The invention is further illustrated in the following non-limiting examples.

Abbreviations used in examples:

AUS: Arthrobacter ureafaciens sialidase
CMP: Cytidine monophosphate
EDTA: Ethylenediaminetetraacetic acid
Gla: (γ)-carboxyglutamic acid
GlcUA: Glucuronic acid

### GlcNAc: N-acetylglucosamine

### Grx2: Glutaredoxin II

### GSH: Glutathione

GSSG: Glutathione disulfide

### HEP: HEParosan

HEP-FX: Heparosan conjugated to Factor X polypeptide (used interchangeably with FX-HEP)
HEP-[N]-FX: HEParosan conjugated via N-glycan to FX.
HEP-[C]-FX: HEParosan conjugated via cysteine to a FX cysteine mutant.
HEP-GSC: GSC-functionalized heparosan polymers
HEP-NH₂: Amine functionalized HEParosan polymer
HEPES: 2-[4-(2-hydroxyethyl)piperazin-1-yl]ethanesulfonic acid

### His: Histidine

### IV: Intravenous

### KO: Knock-out

### MRT: Mean Residence Time

pdFX: Plasma derived human Factor X
PmHS1: Pasteurella mutocida Heparosan Synthase I
pNA: para-nitroaniline
SXa-11: Factor Xa chromogenic substrate
UDP: Uridine diphosphate

### Example 1

### Protein Design of Thrombin Sensitive Factor X Molecules and Nomenclature

### A. Protein Engineering Strategy for Thrombin Sensitive Factor X Molecules

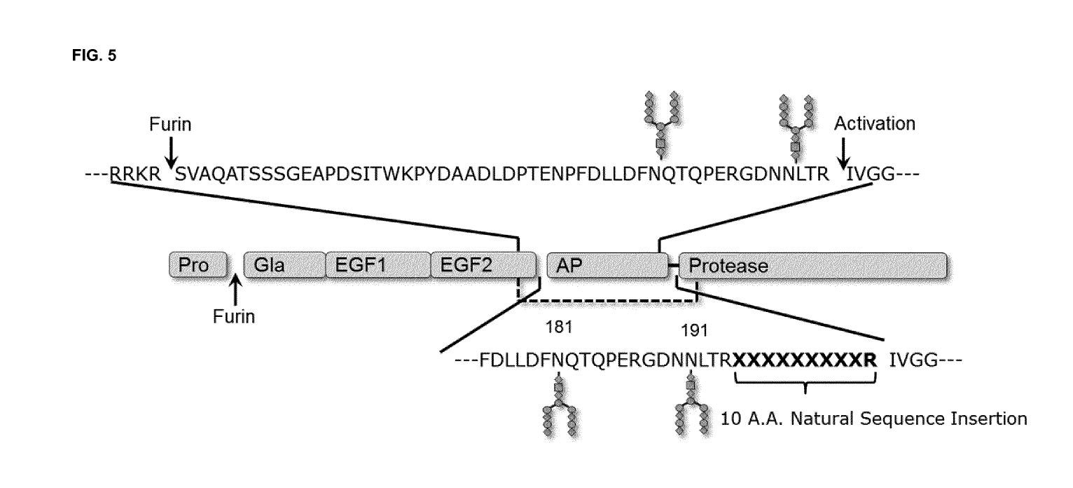

Introduction of thrombin sensitive cleavage sequences into the activation peptide of Factor X was accomplished using the four protein engineering strategies described below. It is known that two N-glycans located on amino acids 181 and 191 of wild type Factor X (SEQ ID NO: 1) are important for maintaining the optimal pharmacokinetic profile of Factor X and modified Factor X molecules (US 2011/0293597). Thus a deliberate factor in all of the design concepts was to retain two N-linked glycosylations sites within the activation peptide, with a preference for preserving the same distance between glycosylation sites. FIGS. 5 through 8 set forth the protein design strategies and illustrate modifications to the wild type Factor X sequence used to generate thrombin sensitive Factor X molecules. As shown in FIGS. 5 through 8, the sequence of Factor X is divided into four different regions, which correspond, according to the mature amino acid sequence numbering system in wild type Factor X (SEQ ID NO: 1) to:
1) The pro-peptide between positions −27 to −1, which is cleaved by furin to release the two-chain 448 amino acid mature zymogen form of Factor X (including the RKR sequence at the C-terminus of the light chain).
2) The light chain between positions 1 to 142, which is comprised of an N-terminal γ-carboxyglutamic acid rich (Gla) domain and two epidermal growth factor (EGF) domains.
3) The activation peptide region located between positions 143 and 194.
4) The heavy chain serine protease domain between positions 195-448. Processing at the Arg¹⁹⁴-Ile¹⁹⁵ peptide bond results in activation of zymogen Factor X to active Factor Xa with an associated release of the 52 amino acid activation peptide.

FIG. 5 illustrates a strategy (hereby designated Strategy 1) where 10 amino acids from the natural thrombin substrate of fibrinopeptide A was inserted directly after the NLTR sequence in wild type Factor X (amino acids 191 to 194; numbering according to the mature amino acid sequence (SEQ ID NO: 1))(cf. also US 2011/0293597). The term “fibrinopeptide A” has its general meaning in the art and refers to a small peptide of 16 amino acids cleaved from the N-terminus of fibrinogen by thrombin. Thrombin sensitive Factor X molecules were designed such that a 10 amino acid sequence (X₁₀-X₁) upstream of thrombin cleavage sites in known substrates were inserted directly after the NLTR sequence in wild type Factor X (amino acids 191 to 194; numbering according to the mature amino acid sequence (SEQ ID NO: 1)) and before the amino acids of the IVGG motif (amino acids 195-198; numbering according to the mature amino acid sequence). All natural inserted sequences are such that the X₁ residue is restricted to arginine (R) giving an inserted sequence of the form X₁₀X₉X₈X₇X₆X₅X₄X₃X₂R₁ where amino acids X₁₀-X₂ were selected from all naturally occurring amino acids: G, A, V, L, I, S, T, C, M, P, Q, N, E, D, K, R, H, F, Y, and W.

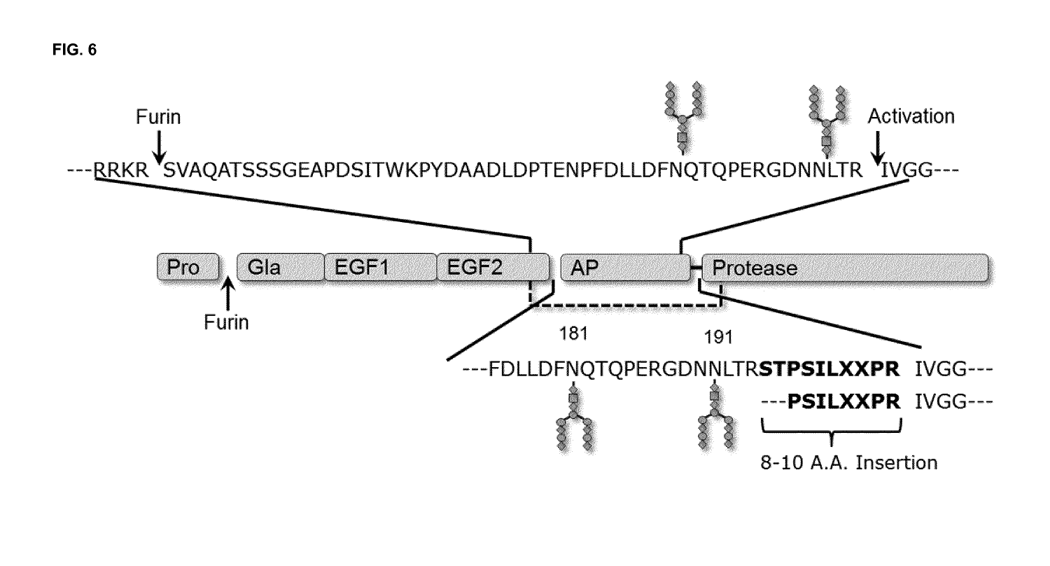

FIG. 6 illustrates a strategy (hereby designated Strategy 2) in which thrombin sensitive Factor X molecules were designed such that an 8-10 amino acid sequence (X₁₀-X₁ or X₈-X₁) was inserted directly after the NLTR sequence in wild type Factor X (amino acids 191 to 194; numbering according to the mature amino acid sequence) and before the amino acids of the IVGG motif (amino acids 195-198; numbering according to the mature amino acid sequence (SEQ ID NO: 1)). All inserted sequences are such that the X₁₀-X₅ or X₈-X₅ amino acids represent the corresponding amino acids N-terminally positioned in relation to the α-thrombin cleavage site in human protease activated receptor 4 (PAR4) where X₁₀-X₁ represent amino acids 21 through 30 in the mature PAR4 sequence (Wu et al. (1998) PNAS, 95: 6642-6646 and Nieman and Schmaier (2007) Biochemistry, 46: 8603-8610). The corresponding insertion sequence was thus of the form S₁₀T₉P₈S₇I₆L₅X₄X₃P₂R₁ or P₈S₇I₆L₅X₄X₃P₂R₁ where amino acids X₄ and X₃ were selected from all naturally occurring amino acids: G, A, V, L, I, S, T, C, M, P, Q, N, E, D, K, R, H, F, Y, and W. The preferred amino acid at X₃ is selected from the following amino acids: Q, M, R, K, T, W, L, I, S and V and is preferably non-negative. The preferred amino acid at X₄ is aliphatic or hydrophobic and selected from the following amino acids: L, I, M, F, V, P and W. Amino acids X₂ and X₁ are restricted to P and R, respectively.

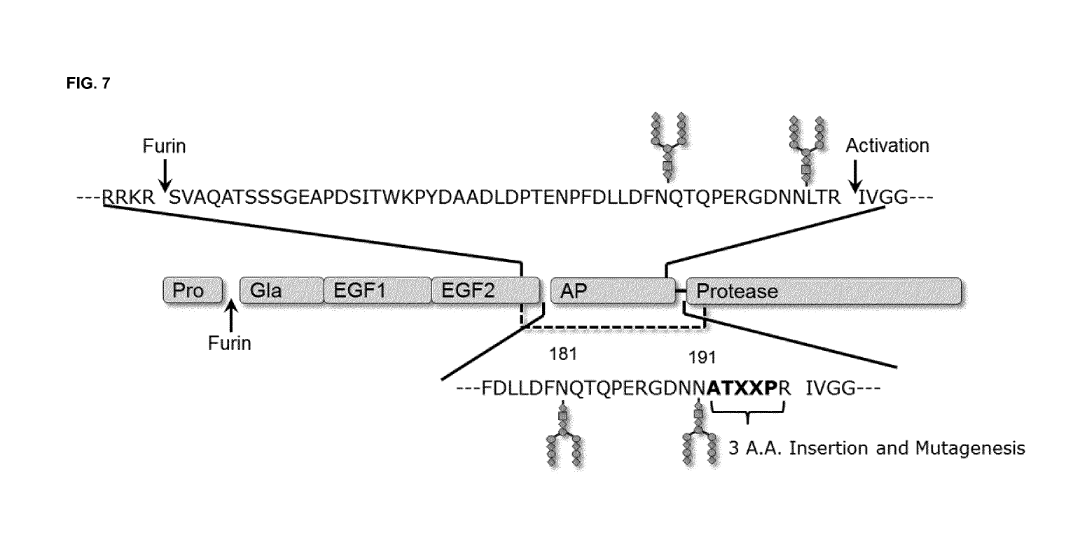

FIG. 7 illustrates a strategy (hereby designated Strategy 3) in which thrombin sensitive Factor X molecules were designed such that the LTR sequence in wild type Factor X (amino acids 192 to 194; numbering according to the mature amino acid sequence (SEQ ID NO: 1)) was replaced by a 6 amino acid sequence (X₆-X₁) of the form A₆T₅X₄X₃P₂R₁ where amino acids X₄ and X₃ were selected from all naturally occurring amino acids: G, A, V, L, I, S, T, C, M, P, Q, N, E, D, K, R, H, F, Y, and W. The preferred amino acid at X₃ is selected from the following amino acids: Q, M, R, K, T, W, L, I, S and V and is preferably non-negative. The preferred amino acid at X₄ is aliphatic or hydrophobic and selected from the following amino acids: L, I, M, F, V, P and W. Amino acids X₂ and X₁ are restricted to P and R, respectively with the R¹⁹⁴ (X₁) being unmodified from the original sequence. In order to preserve an N-glycosylation motif of N×T/S and full glycosylation of N¹⁹¹ (X₇), X₆ and X₅ are fixed as A and T, respectively. This protein design approach minimizes the alterations to the natural Factor X sequence of the activation peptide such that the final construct is fully embodied by a three amino acid insert and two amino acid mutagenesis as set forth in the following exemplar: insertion of A₆T₅X₄ and mutagenesis of L¹⁹² and T¹⁹³ to X₃P₂ with retention of R¹⁹⁴ as R₁.

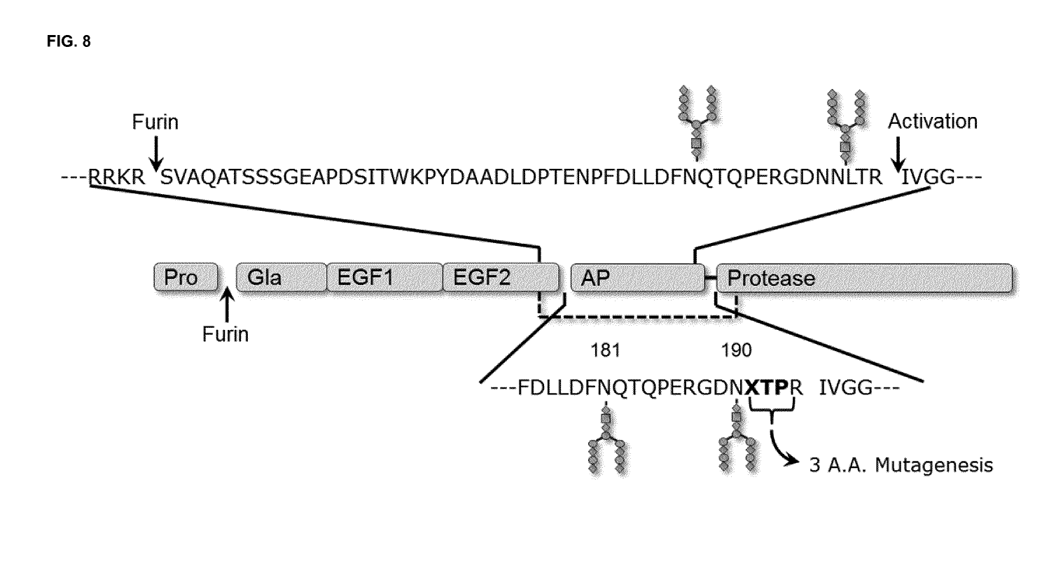

FIG. 8 illustrates a strategy (hereby designated Strategy 4) in which thrombin sensitive Factor X molecules were designed such that the NLTR sequence in wild type Factor X (amino acids 191 to 194; numbering according to the mature amino acid sequence (SEQ ID NO: 1)) is replaced by a 4 amino acid sequence (X₄-X₁) of the form X₄T₃P₂R₁ where the amino acids acid X₄ was selected from naturally occurring amino acids: G, A, V, L, I, S, T, C, M, P, Q, N, E, D, K, R, H, F, Y, and W. The preferred amino acid at X₄ is aliphatic or hydrophobic and selected from the following amino acids: L, I, M, F, V, P and W. Amino acids X₃, X₂ and X₁ are restricted to T, P and R, respectively with the R¹⁹⁴ (X₁) being unmodified from the original sequence. In order to preserve an N-glycosylation motif of N×T/S, X₃ was fixed as T such that an N-linked glycosylation site is introduced at N¹⁹⁰ (X₅). This protein design approach minimizes the alterations to the natural Factor X sequence of the activation peptide such that the final construct is fully embodied by three amino acid modifications as set forth in the following exemplar: mutagenesis of N¹⁹¹, L¹⁹² and T¹⁹³ to X₄T₃P₂ with retention of R¹⁹⁴ as R₁.

### B. Nomenclature for the Naming of Thrombin Sensitive Factor X Molecules

Exemplary thrombin sensitive Factor X molecules provided herein are designated by the following naming nomenclature, which relates to the protein design strategies discussed above in part A. For thrombin sensitive Factor X molecules prepared by either Strategy 1 or Strategy 2 the nomenclature used throughout adhere to the following general terminology: FX ins[194]>[X₁₀X₉X₈X₇X₆X₅X₄X₃X₂X₁], where FX ins[194] refers to the placement of the inserted peptide sequence after amino acid 194 in wild type Factor X (SEQ ID NO: 1) and [X₁₀X₉X₈X₇X₆X₅X₄X₃X₂X₁] or [X₈X₇X₆X₅X₄X₃X₂X₁] refer to the single letter designation amino acid sequence which has been inserted into the activation peptide between R¹⁹⁴ and I¹⁹⁵ of wild type Factor X (SEQ ID NO: 1). For thrombin sensitive Factor X molecules prepared by Strategy 3 the nomenclature used throughout adhere to the following general terminology: FX [191-194]>[X₆X₅X₄X₃X₂X₁], where FX [191-194] refers to substitution of the four amino acids inclusive of the N¹⁹¹ to R¹⁹⁴ sequence of wild type Factor X (SEQ ID NO: 1) with a six amino acid sequence of [X₆X₅X₄X₃X₂X₁] referred to by its single letter amino acid designation. For thrombin sensitive Factor X molecules prepared by Strategy 4 the nomenclature used throughout adhere to the following general terminology: FX [191-194]>[X₄X₃X₂X₁], where FX [191-194] refers to substitution of the four amino acids inclusive of the N¹⁹¹ to R¹⁹⁴ sequence of wild type Factor X (SEQ ID NO: 1) with a four amino acid sequence of [X₄X₃X₂X₁] referred to by its single letter amino acid designation. In particular examples, modified thrombin sensitive Factor X molecules provided herein have further modifications in which a C-terminal HPC4 tag (-HPC4) has been added for purposes of purification (where the term “HPC4” has its general meaning in the art and refers to a small peptide of 11 amino acids, DQVDPRLIDGK, from Protein C used as an affinity purification tag) or the N-terminal γ-carboxyglutamic acid rich (Gla) domain defined by amino acids 1-47 of wild type Factor X (SEQ ID NO: 1) has been deleted (desGla-). Hence, modified thrombin sensitive molecules provided herein can be further described by appending their naming nomenclature with defined N-terminal (desGla-) or C-terminal (-HPC4) modifications.

Table 1 sets forth the thrombin sensitive Factor X molecules that were generated, with nomenclature indicating the modification to create a thrombin sensitive molecule and discussed herein. The provided SEQ ID NOs refer to the listed Factor X molecules. Also listed are the thrombin cleavage sequences (X₄-X_{4′}), wherein the cleavage occurs between X₁ and X_{1′}.

TABLE 1
Thrombin Sensitive Factor X Molecules

| | SEQ | | SEQ | X₄-X_{4′} |
|---|---|---|---|---|
| | ID | Experimental Compound | ID | Cleavage |
| Compound Name | NO | Name | NO | Sequence |
| FX | 1 | FX-HPC4 | 12 | NLTR-IVGG |
| FX | 3 | FX ins[194] > [DFLAEGGGVR]- | 6 | GGVR-IVGG |
| ins[194] > [DFLAEGGGVR] | | HPC4 | | |
| FX | 3 | desGla-FX | 226 | GGVR-IVGG |
| ins[194] > [DFLAEGGGVR] | | ins[194] > [DFLAEGGGVR]- | | |
| | | HPC4 | | |
| FX | 8 | desGla-FX | 228 | GGPR-IVGG |
| ins[194] > [DFLAEGGGPR] | | ins[194] > [DFLAEGGGPR]- | | |
| | | HPC4 | | |
| FX | 8 | FX ins[194] > [DFLAEGGGPR]- | 10 | GGPR-IVGG |
| ins[194] > [DFLAEGGGPR] | | HPC4 | | |
| FX [191-194] > [NATLMPR] | 16 | FX [191-194] > [NATLMPR]- | 14 | LMPR-IVGG |
| | | HPC4 | | |
| FX [191-194] > [NATLRPR] | 20 | FX [191-194] > [NATLRPR]- | 18 | LRPR-IVGG |
| | | HPC4 | | |
| FX [191-194] > [NATMMPR] | 24 | FX [191-194] > [NATMMPR]- | 22 | MMPR-IVGG |
| | | HPC4 | | |
| FX [191-194] > [NATMRPR] | 28 | FX [191-194] > [NATMRPR]- | 26 | MRPR-IVGG |
| | | HPC4 | | |
| FX [191-194] > [NATMTPR] | 32 | FX [191-194] > [NATMTPR]- | 30 | MTPR-IVGG |
| | | HPC4 | | |
| FX [191-194] > [NATIQPR] | 36 | FX [191-194] > [NATIQPR]-HPC4 | 34 | IQPR-IVGG |
| FX [191-194] > [NATIMPR] | 40 | FX [191-194] > [NATIMPR]-HPC4 | 38 | IMPR-IVGG |
| FX [191-194] > [NATIRPR] | 44 | FX [191-194] > [NATIRPR]-HPC4 | 42 | IRPR-IVGG |
| FX [191-194] > [NATITPR] | 48 | FX [191-194] > [NATITPR]-HPC4 | 46 | ITPR-IVGG |
| FX [191-194] > [NATFRPR] | 52 | FX [191-194] > [NATFRPR]- | 50 | FRPR-IVGG |
| | | HPC4 | | |
| FX [191-194] > [NATLSPR] | 56 | FX [191-194] > [NATLSPR]-HPC4 | 54 | LSPR-IVGG |
| FX [191-194] > [NATLDPR] | 60 | FX [191-194] > [NATLDPR]- | 58 | LDPR-IVGG |
| | | HPC4 | | |
| FX [191-194] > [NATLQPR] | 64 | FX [191-194] > [NATLQPR]- | 62 | LQPR-IVGG |
| | | HPC4 | | |
| FX [191-194] > [NATLTPR] | 68 | FX [191-194] > [NATLTPR]-HPC4 | 66 | LTPR-IVGG |
| FX [191-194] > [NATMQPR] | 72 | FX [191-194] > [NATMQPR]- | 70 | MQPR-IVGG |
| | | HPC4 | | |
| FX [191-194] > [NATIKPR] | 76 | FX [191-194] > [NATIKPR]-HPC4 | 74 | IKPR-IVGG |
| FX [191-194] > [NATLEPR] | 80 | FX [191-194] > [NATLEPR]-HPC4 | 78 | LEPR-IVGG |
| FX [191-194] > [NATDTPR] | 84 | FX [191-194] > [NATDTPR]- | 82 | DTPR-IVGG |
| | | HPC4 | | |
| FX [191-194] > [NATLKPR] | 88 | FX [191-194] > [NATLKPR]-HPC4 | 86 | LKPR-IVGG |
| FX [191-194] > [NATFTPR] | 92 | FX [191-194] > [NATFTPR]-HPC4 | 90 | FTPR-IVGG |
| FX [191-194] > [NATFKPR] | 96 | FX [191-194] > [NATFKPR]- | 94 | FKPR-IVGG |
| | | HPC4 | | |
| FX [191-194] > [NATMKPR] | 100 | FX [191-194] > [NATMKPR]- | 98 | MKPR-IVGG |
| | | HPC4 | | |
| FX [191-194] > [NATWQPR] | 104 | FX [191-194] > [NATWQPR]- | 102 | WQPR-IVGG |
| | | HPC4 | | |
| FX [191-194] > [LTPR] | 108 | FX [191-194] > [LTPR]-HPC4 | 106 | LTPR-IVGG |
| FX [191-194] > [MTPR] | 112 | FX [191-194] > [MTPR]-HPC4 | 110 | MTPR-IVGG |
| FX [191-194] > [ITPR] | 116 | FX [191-194] > [ITPR]-HPC4 | 114 | ITPR-IVGG |
| FX [191-194] > [FTPR] | 120 | FX [191-194] > [FTPR]-HPC4 | 118 | FTPR-IVGG |
| FX | 124 | FX ins[194] > [STPSILLKPR]- | 122 | LKPR-IVGG |
| ins[194] > [STPSILLKPR] | | HPC4 | | |
| FX | 128 | FX ins[194] > [STPSILFTPR]- | 126 | FTPR-IVGG |
| ins[194] > [STPSILFTPR] | | HPC4 | | |
| FX | 132 | FX ins[194] > [STPSILFKPR]- | 130 | FKPR-IVGG |
| ins[194] > [STPSILFKPR] | | HPC4 | | |
| FX | 136 | FX ins[194] > [STPSILMKPR]- | 134 | MKPR-IVGG |
| ins[194] > [STPSILMKPR] | | HPC4 | | |
| FX | 140 | FX ins[194] > [STPSILWQPR]1 | 138 | WQPR-IVGG |
| ins[194] > [STPSILWQPR] | | HPC4 | | |
| FX | 144 | FX ins[194] > [STPSILLRPR]- | 142 | LRPR-IVGG |
| ins[194] > [STPSILLRPR] | | HPC4 | | |
| FX | 148 | FX ins[194] > [STPSILMTPR]- | 146 | MTPR-IVGG |
| ins[194] > [STPSILMTPR] | | HPC4 | | |
| FX ins[194] > [PSILLKPR] | 152 | FX ins[194] > [PSILLKPR]-HPC4 | 150 | LKPR-IVGG |
| FX ins[194] > [PSILFTPR] | 156 | FX ins[194] > [PSILFTPR]-HPC4 | 154 | FTPR-IVGG |
| FX ins[194] > [PSILFKPR] | 160 | FX ins[194] > [PSILFKPR]-HPC4 | 158 | FKPR-IVGG |
| FX ins[194] > [PSILMKPR] | 164 | FX ins[194] > [PSILMKPR]-HPC4 | 162 | MKPR-IVGG |
| FX ins[194] > [PSILWQPR] | 168 | FX ins[194] > [PSILWQPR]-HPC4 | 166 | WQPR-IVGG |
| FX ins[194] > [PSILLRPR] | 172 | FX ins[194] > [PSILLRPR]-HPC4 | 170 | LRPR-IVGG |
| FX ins[194] > [PSILMTPR] | 176 | FX ins[194] > [PSILMTPR]-HPC4 | 174 | MTPR-IVGG |
| FX | 180 | desGla-FX | 178 | FNPR-IVGG |
| ins[194] > [SEYQTFFNPR] | | ins[194] > [SEYQTFFNPR]-HPC4 | | |
| FX | 184 | desGla-FX | 182 | IKPR-IVGG |
| ins[194] > [NESTTKIKPR] | | ins[194] > [NESTTKIKPR]-HPC4 | | |
| FX | 188 | desGla-FX | 186 | PAPR-IVGG |
| ins[194] > [STPSILPAPR] | | ins[194] > [STPSILPAPR]-HPC4 | | |
| FX | 192 | desGla-FX | 190 | VVPR-IVGG |
| ins[194] > [TVELQGVVPR] | | ins[194] > [TVELQGVVPR]-HPC4 | | |
| FX | 196 | desGla-FX | 194 | IQIR-IVGG |
| ins[194] > [DNSPSFIQIR] | | ins[194] > [DNSPSFIQIR]-HPC4 | | |
| FX | 200 | desGla-FX | 198 | FSAR-IVGG |
| ins[194] > [DNEEGFFSAR] | | ins[194] > [DNEEGFFSAR]-HPC4 | | |
| FX | 204 | desGla-FX | 202 | WYLR-IVGG |
| ins[194] > [PDNIAAVVYLR] | | ins[194] > [PDNIAAWYLR]-HPC4 | | |
| FX | 208 | desGla-FX | 206 | IEPR-IVGG |
| ins[194] > [LSKNNAIEPR] | | ins[194] > [LSKNNAIEPR]-HPC4 | | |
| FX | 212 | desGla-FX | 210 | QSPR-IVGG |
| ins[194] > [YDEDENQSPR] | | ins[194] > [YDEDENQSPR]- | | |
| | | HPC4 | | |
| FX | 216 | desGla-FX | 214 | LSPR-IVGG |
| ins[194] > [HTHHAPLSPR] | | ins[194] > [HTHHAPLSPR]-HPC4 | | |
| FX | 220 | desGla-FX | 218 | LGIR-IVGG |
| ins[194] > [NRLAAALGIR] | | ins[194] > [NRLAAALGIR]-HPC4 | | |
| FX | 224 | desGla-FX | 222 | LDPR-IVGG |
| ins[194] > [KATNATLDPR ] | | ins[194] > [KATNATLDPR ]-HPC4 | | |
| FX [191- | 232 | FX [191- | 230 | LDPR-IVGG |
| 194] > [GGGSGGGKEEEDI | | 194] > [GGGSGGGKEEEDIEFEE | | |
| EFEEFESSPKPDGSGGG | | FESSPKPDGSGGGSGGGNAT | | |
| SGGGNATLDPR] | | LDPR]-HPC4 | | |
| FX [191- | 236 | FX [191- | 234 | LDPR-IVGG |
| 194] > [GGGSGGGSGDPK | | 194] > [GGGSGGGSGDPKPSSE | | |
| PSSEFEEFEIDEEEKGGG | | FEEFEIDEEEKGGGSGGGNAT | | |
| SGGGNATLDPR] | | LDPR]-HPC4 | | |

### Example 2

### Generation of the Quenched Fluorescence Peptide Substrate Library

### A. Library Construction and Synthesis

Solid phase resin Pal-ChemMatrix was purchased by PCAS BioMatrix and all Fmoc-amino acid were from Protein technologies, except for Fmoc-Lys(Dnp)-OH (IRIS Gmbh, Germany) and Fmoc-Lys(retro Boc)Abz (Bachem). Oxyma Pure was purchased from Merck (Switzerland) N-methyl-pyrrolidinone (NMP), diisopropylcarbodiimide (DIC), trifluoroacetic acid (TFA) were peptide grade and obtained from Biosolve (Netherlands).

A quenched fluorescence peptide substrate library using an o-aminobenzoic acid (Abz) fluorescence donor and a 2,4-dinitrophenyl (Dnp) quencher moiety with the amino acid sequence of Lys(Dnp)-ATNATX₄X₃PRIVGG-Lys(Abz) (SEQ ID NO: 237) was constructed by randomizing every possible natural amino acid combination in X₄ and X₃ with the exception of cysteine. The quenched fluorescence peptide substrates (QF-substrates) were synthesized by a standard Fmoc-strategy on Multipep RS (Intavis, Germany) in 96-well microtiter filter plates (Nunc). In each well was distributed 15 mg resin and three couplings were done in each synthesis cycle. A single coupling step consisted of adding to each well 90 μL Fmoc-amino acid (0.3 M in NMP containing 0.3 M Oxymapure)+30 μL DIC and 30 μL collidine. Before adding the amino acids to the resin, they were preactivated in a mixer vial according to the multipep RS manufacturer instructions. The first coupling step was coupled for 15 minutes, coupling step 2 was coupled for 1 hour and coupling step three was coupled for 3 hours. After coupling step 3, the resin was washed using 300 μL NMP to each well five times using the manifold as described by the manufacture. A deprotection step of the Fmoc group was accomplished by adding 200 μL 25% piperidine twice to each well. The first deprotection step was allowed to proceed for 2 minutes and the second step was allowed to proceed for 8 minutes. After the last deprotection step the resin was washed as previously described.

After the solid phase synthesis reaction, the resin was washed 7 times with ethanol by adding 300 μL to each well. The resin was allowed to dry overnight and subsequently was deprotected with 4% triisopropylsilane, 1% thioanisol and 3% H₂O in 92% TFA. This was done by placing the filter plate on top of a 2 mL deep-well collector plate. Then 250 μL TFA was added to each well and the TFA was allowed to flow through. After 2 minutes this was repeated and after 5 min another 250 μL was added and allowed to stand for 1-2 hours. The resin was washed with 2×250 μL TFA (as described above) and the collected TFA was concentrated to approximately 100-150 μL by argon flow. The peptides were precipitated with diethyl ether and transferred to a filter plate (Solvinert, Millipore) and the precipitated peptides were washed with diethyl ether five times. A Solvinert filter plate was placed on top of a 2 ml deep-well plate (master plate) and the peptides were dissolved in 80% DMSO (in H₂O). The filter plates were shaken gently overnight and then the peptides were transferred to the master plate by evacuation in a Waters vacuum manifold. Five randomly chosen peptides from each of the four library plates were analysed by MALDI and the identity confirmed.

### B. Determination of the Stock Concentration for Quenched Fluorescence Peptide Substrates

Quenched fluorescence substrate (QF-substrate) samples synthesized in house (described above) or by an external supplier (Aurigene, Bangalore, India) were typically stored in 80% DMSO or resuspended from a lyophilized powder in 100% DMSO, respectively. The molar concentration of a stock of QF peptide substrate was typically determined from the absorbance of the 2,4-dinitrophenyl (Dnp) quencher moiety by one of the two following two methods. In the first example, the stock concentration was determined directly from the absorbance of the QF-substrate peptide solution at 365 nm using an extinction coefficient of 17,300 M⁻¹ cm⁻¹ for the Dnp quencher moiety (Carmona et al. (2006) Nature Protocols 1: 1971-1976). In order to determine the concentration, stock samples (˜5-20 mM) were serially diluted in fresh DMSO 1:10 and 1:100 in a 96-well polypropylene plate. The Nanodrop-1000 was used to quantitate the absorbance of a 2 μL sample of the 1:100 or 1:10 dilution to achieve an absorbance reading of 0.1-0.8 AU in UV/Vis mode with a 1 nm path length. Readings were acquired in duplicate from independently diluted samples and averaged. The concentration of a QF-substrate (in mM) was then determined from the following equation which corrects for the 1 mm path length:

[QF-Substrate]=((Abs₃₆₅×dilution×10)/Ext. Coefficient)×1000

The QF-substrate libraries were typically prepared to a stock concentration of 4500 μM (i.e. 4.5 mM). Each substrate plate (96-well) was diluted to an estimated concentration of 500 μM in 100% DMSO (10 μL of stock+80 μL of DMSO). This dilution was used to prepare a dilution plate for quantification by mixing 40 μL with 60 μL of assay buffer (50 mM Hepes, 150 mM NaCl, 10 mM CaCl₂, 0.1% PEG8000, pH 7.4). The absorbance at 365 nm of the diluted QF peptide substrate stock was quantified using a Molecular Devices absorbance spectrometer with duplicate readings that were averaged. The concentration of each QF peptide substrate was subsequently confirmed by comparison to a standard curve (0 to 450 nM) of a control QF peptide substrate diluted in 50% DMSO/assay buffer. The concentration of the control QF peptide stock solution was determined directly from the absorbance at 365 nm as described above.

### Example 3

### Screening the Quenched Fluorescence Peptide Library to Evaluate the Kinetic Rate Constant for Thrombin Cleavage

In order to determine the cleavage rates for the QF peptide substrate library, a progress curve protocol was designed for evaluating the kinetics of substrate cleavage by thrombin. The progress curve method assumed that the reaction followed a simple Michaelis Menten mechanism with the encounter complex of substrate and enzyme being limiting (i.e. psuedo-1^{st} order). Under conditions where [QF-substrate]<<K_{M} this method allowed for an estimation of the k_{cat}/K_{M} from an exponential fit of the complete reaction progress curves (i.e. complete substrate hydrolysis over time). The stock concentrations of each QF substrate (up to 96 per plate) were confirmed to be in the range of ˜3500-4500 μM using the plate method described in Example 2 above. To initiate the cleavage reactions, the QF peptide substrates (in a 96-well format) were first diluted to ˜500 μM in 100% DMSO by mixing 10 μL of stock substrate+80 μL DMSO followed by two subsequent serial dilutions with assay buffer (50 mM Hepes, 150 mM NaCl, 10 mM CaCl₂, 0.1% PEG8000, pH 7.4) taking 20 μl of dilution 1+80 μL assay buffer (˜100 μM in 20% DMSO) and then 20 μL of dilution 2+180 μL assay buffer (˜10 μM in 2% DMSO). Human plasma purified α-thrombin was diluted from the stock to a working concentration of 1 μM in assay buffer. Progress curve reactions were initiated by combining 100 μL of QF substrate dilution three (˜10 μM in 2% DMSO) with 80 μL of assay buffer and 20 μL of 1 μM thrombin in a 96-well black assay plate. Reactions were followed in a Molecular Devices fluorescence spectrometer for 3 hours at 37° C. using an excitation wavelength of 320 nm and an emission wavelength of 420 nm without any cut-off filter.

Data collected using the SoftMax Pro software were exported as .txt files for analysis using Excel analysis templates and non-linear regression analysis using the GraphPad/Prism software suite. Progress curves were fit to the following equation:

Y=F₀+Fₘₐₓ*(1−exp(−E*k*x))

where x=reaction time, F₀=the initial fluorescence intensity, Fₘₐₓ=the maximum fluorescence intensity at complete hydrolysis, k=the kinetic rate constant in the form of k_{cat}/K_{M} with the units of M⁻¹s⁻¹ and E=the enzyme concentration in M units.

Table 2 and Table 3 set forth the data generated from screening the quenched fluorescence positional scanning library and a set of rationally designed quenched fluorescence substrates based on natural thrombin cleavage sequences, respectively. The quenched fluorescence positional scanning library (X₄/X₃) was based on the PAR 1 thrombin cleavage sequence (table 2), however, using a fixed prime side sequence of IVGG, such that the library is of the form Lys(Dnp)-ATNATX₄X₃PRIVGG-Lys(Abz), where Lys(Dnp) and Lys(Abz) (SEQ ID NO: 238) are the fluorescence quencher and donor moieties, respectively. The rationally designed QF-substrates based on natural thrombin cleavage sequences (Table 3) were synthesized by Aurigene (Bangalore, India) and also contained a fixed prime side sequence of IVGG, such that the library is of the form Lys(Dnp)-X₁₀X₉X₈X₇X₆X₅X₄X₃X₂X₁IVGG-Lys(Abz), where Lys(Dnp) and Lys(Abz) (SEQ ID NO: 239) are the fluorescence quencher and donor moieties, respectively. Data are presented in Tables 2 and 3 as the ranked k_{cat}/K_{M} rate constants, standard deviation, % CV as well as the fold improvement over the PAR 1 control substrate sequence [Lys(Dnp)-ATNATLDPRIVGG-Lys(Abz)] (SEQ ID NO: 241) and the Fibrinopeptide A (FpA) substrate sequence [Lys(Dnp)-DFLAEGGGVRIVGG-Lys(Abz)] (SEQ ID NO: 240).

At least 20 sequences were selected from the QF-substrate library with >20-fold improved cleavage rates (k_{cat}/K_{M}) over the parent PAR-1 sequence and up to 120-fold improved cleavage rates over the FpA substrate sequence. Likewise, several natural thrombin sequences (Table 3) demonstrated 5 to 14-fold improved cleavage rates of that of the PAR-1 control and up to 100-fold improved cleavage rates over the FpA control substrate sequence. The most improved natural substrate was shown to be the FpA_P sequence, which has a proline residue at X₂ instead of the naturally occurring valine. Based on the results of the QF-substrate library screenings, the following preferred sequence motifs were determined for X₄, X₃ and X₂ with a fixed X₁ amino acid of arginine (R). The preferred amino acid in X₂ is proline (P), while the preferred amino acid in X₃ is fairly flexible and selected from Q, M, R, T, W, K, I or V, but is not negative or proline. The preferred amino acid in position X₄ is more restricted, being mostly aliphatic or hydrophobic and selected from L, I, M, F, V, P or W, but is not charged or selected from G, S or T.

TABLE 2
X₄/X₃ Positional Scanning Quenched Fluorescence Library
X₄/X₃ Positional Scanning Quenched Fluorescence Library:
form Lys(Dnp)-ATNATX₄X₃PRIVGG-Lys(Abz), where Lys(Dnp)
and Lys(Abz) (SEQ ID NO: 238) are the fluorescence quencher
and donor moieties, respectively. All amino acid variants
herein can form part of FX molecules according to the invention.

| | | | | | | Fold- | Fold- |
|---|---|---|---|---|---|---|---|
| | | k_{cat}/K_{M} | | | | Improved | Improved |
| X₄ | X₃ | (M⁻¹s⁻¹) | S.D. | % CV | n = | (PAR-1) | (FpA) |
| L | Q | 4.9E+04 | 4.9E+03 | 10% | 2 | 17.4 | 119.6 |
| L | M | 4.7E+04 | 2.2E+03 | 5% | 2 | 16.6 | 114.4 |
| L | R | 4.5E+04 | 6.0E+03 | 13% | 2 | 16.0 | 110.4 |
| I | Q | 4.4E+04 | 2.7E+03 | 6% | 2 | 15.7 | 108.2 |
| L | T | 4.4E+04 | 1.0E+03 | 2% | 2 | 15.6 | 107.7 |
| F | R | 4.3E+04 | 5.0E+03 | 12% | 2 | 15.4 | 106.5 |
| I | M | 4.1E+04 | 1.5E+03 | 4% | 2 | 14.6 | 100.8 |
| I | R | 3.7E+04 | 1.3E+02 | 0% | 2 | 13.2 | 91.2 |
| L | W | 3.6E+04 | 2.1E+02 | 1% | 2 | 13.0 | 89.6 |
| M | R | 3.5E+04 | 1.0E+03 | 3% | 2 | 12.3 | 85.1 |
| L | I | 3.4E+04 | 2.1E+03 | 6% | 2 | 12.0 | 82.7 |
| I | W | 3.3E+04 | 1.8E+03 | 5% | 2 | 11.8 | 81.2 |
| M | Q | 3.3E+04 | 8.7E+02 | 3% | 2 | 11.6 | 80.0 |
| M | T | 3.2E+04 | 1.9E+03 | 6% | 2 | 11.4 | 78.7 |
| I | T | 3.2E+04 | 1.2E+03 | 4% | 2 | 11.3 | 78.1 |
| L | K | 3.2E+04 | 2.3E+03 | 7% | 2 | 11.3 | 77.6 |
| F | Q | 3.0E+04 | 1.8E+03 | 6% | 2 | 10.8 | 74.6 |
| L | V | 3.0E+04 | 3.8E+02 | 1% | 2 | 10.5 | 72.6 |
| M | M | 2.8E+04 | 5.7E+02 | 2% | 2 | 9.9 | 67.9 |
| F | T | 2.7E+04 | 1.2E+03 | 5% | 2 | 9.6 | 66.3 |
| P | W | 2.7E+04 | 3.8E+02 | 1% | 2 | 9.5 | 65.4 |
| P | Q | 2.6E+04 | 1.5E+03 | 6% | 2 | 9.4 | 64.6 |
| I | I | 2.6E+04 | 1.9E+03 | 7% | 2 | 9.4 | 64.5 |
| I | H | 2.6E+04 | 1.3E+03 | 5% | 2 | 9.2 | 63.6 |
| L | H | 2.5E+04 | 5.8E+02 | 2% | 2 | 9.0 | 61.7 |
| F | K | 2.5E+04 | 1.6E+03 | 7% | 2 | 8.9 | 61.6 |
| F | M | 2.4E+04 | 1.4E+03 | 6% | 2 | 8.5 | 58.6 |
| I | V | 2.4E+04 | 1.4E+03 | 6% | 2 | 8.4 | 58.2 |
| I | K | 2.4E+04 | 4.6E+02 | 2% | 2 | 8.4 | 58.2 |
| V | W | 2.3E+04 | 7.2E+02 | 3% | 2 | 8.2 | 56.3 |
| W | R | 2.3E+04 | 2.0E+02 | 1% | 2 | 8.1 | 55.5 |
| M | K | 2.3E+04 | 4.7E+02 | 2% | 2 | 8.0 | 55.4 |
| P | T | 2.2E+04 | 2.6E+01 | 0% | 2 | 7.7 | 53.1 |
| L | S | 2.1E+04 | 3.4E+02 | 2% | 2 | 7.5 | 51.4 |
| V | Q | 2.1E+04 | 5.0E+02 | 2% | 2 | 7.4 | 51.3 |
| L | Y | 2.1E+04 | 6.9E+02 | 3% | 2 | 7.4 | 51.2 |
| F | W | 2.0E+04 | 1.1E+03 | 6% | 2 | 7.0 | 48.5 |
| A | R | 2.0E+04 | 3.4E+02 | 2% | 2 | 7.0 | 48.0 |
| W | Q | 1.9E+04 | 8.8E+02 | 5% | 2 | 6.8 | 47.0 |
| M | S | 1.8E+04 | 7.8E+02 | 4% | 2 | 6.6 | 45.3 |
| I | Y | 1.8E+04 | 2.3E+01 | 0% | 2 | 6.4 | 44.3 |
| P | I | 1.8E+04 | 2.8E+02 | 2% | 2 | 6.4 | 44.0 |
| M | W | 1.8E+04 | 2.8E+03 | 16% | 2 | 6.3 | 43.7 |
| M | H | 1.8E+04 | 6.7E+02 | 4% | 2 | 6.3 | 43.5 |
| M | I | 1.8E+04 | 5.3E+02 | 3% | 2 | 6.2 | 43.0 |
| F | H | 1.7E+04 | 9.5E+02 | 6% | 2 | 6.1 | 42.2 |
| P | R | 1.7E+04 | 5.5E+02 | 3% | 2 | 6.0 | 41.3 |
| M | V | 1.7E+04 | 1.9E+02 | 1% | 2 | 6.0 | 41.2 |
| W | S | 1.6E+04 | 7.8E+01 | 0% | 2 | 5.8 | 40.3 |
| W | T | 1.6E+04 | 1.1E+02 | 1% | 2 | 5.8 | 40.1 |
| V | R | 1.6E+04 | 1.5E+02 | 1% | 2 | 5.8 | 39.7 |
| A | T | 1.6E+04 | 1.3E+03 | 8% | 2 | 5.8 | 39.7 |
| V | T | 1.6E+04 | 7.8E+02 | 5% | 2 | 5.6 | 38.9 |
| L | L | 1.6E+04 | 7.4E+02 | 5% | 2 | 5.6 | 38.7 |
| L | F | 1.5E+04 | 2.1E+03 | 14% | 2 | 5.4 | 36.9 |
| V | M | 1.5E+04 | 6.2E+02 | 4% | 2 | 5.3 | 36.8 |
| A | W | 1.5E+04 | 1.6E+03 | 11% | 2 | 5.3 | 36.3 |
| I | L | 1.5E+04 | 3.9E+02 | 3% | 2 | 5.2 | 35.9 |
| P | H | 1.5E+04 | 3.6E+02 | 2% | 2 | 5.2 | 35.8 |
| L | E | 1.4E+04 | 9.5E+02 | 7% | 2 | 5.0 | 34.2 |
| P | Y | 1.4E+04 | 1.6E+02 | 1% | 2 | 5.0 | 34.1 |
| F | G | 1.4E+04 | 5.5E+02 | 4% | 2 | 4.9 | 33.9 |
| I | S | 1.4E+04 | 1.9E+03 | 14% | 2 | 4.9 | 33.8 |
| P | M | 1.4E+04 | 5.4E+02 | 4% | 2 | 4.9 | 33.8 |
| L | N | 1.4E+04 | 7.2E+02 | 5% | 2 | 4.9 | 33.6 |
| W | A | 1.3E+04 | 3.5E+02 | 3% | 2 | 4.7 | 32.5 |
| I | F | 1.3E+04 | 1.8E+02 | 1% | 2 | 4.7 | 32.3 |
| P | V | 1.3E+04 | 1.4E+02 | 1% | 2 | 4.7 | 32.2 |
| W | K | 1.3E+04 | 1.9E+02 | 1% | 2 | 4.7 | 32.2 |
| V | I | 1.3E+04 | 3.5E+02 | 3% | 2 | 4.7 | 32.2 |
| L | A | 1.3E+04 | 1.0E+02 | 1% | 2 | 4.6 | 31.9 |
| V | H | 1.3E+04 | 5.7E+02 | 4% | 2 | 4.6 | 31.6 |
| A | Q | 1.2E+04 | 2.3E+01 | 0% | 2 | 4.3 | 29.8 |
| M | Y | 1.2E+04 | 4.6E+02 | 4% | 2 | 4.3 | 29.7 |
| A | I | 1.2E+04 | 9.0E+02 | 8% | 2 | 4.3 | 29.3 |
| P | S | 1.2E+04 | 3.6E+02 | 3% | 2 | 4.2 | 28.7 |
| F | Y | 1.2E+04 | 2.8E+02 | 2% | 2 | 4.2 | 28.7 |
| M | A | 1.2E+04 | 5.3E+02 | 5% | 2 | 4.1 | 28.6 |
| I | E | 1.2E+04 | 5.5E+02 | 5% | 2 | 4.1 | 28.5 |
| A | Y | 1.1E+04 | 4.1E+03 | 36% | 2 | 4.1 | 28.1 |
| W | G | 1.1E+04 | 4.7E+02 | 4% | 2 | 4.1 | 28.1 |
| V | Y | 1.1E+04 | 6.6E+02 | 6% | 2 | 3.9 | 27.2 |
| V | V | 1.1E+04 | 6.0E+02 | 6% | 2 | 3.9 | 26.8 |
| M | F | 1.1E+04 | 1.4E+02 | 1% | 2 | 3.8 | 26.4 |
| M | L | 1.1E+04 | 1.2E+03 | 12% | 2 | 3.8 | 26.2 |
| A | K | 1.1E+04 | 5.0E+02 | 5% | 2 | 3.8 | 26.2 |
| I | N | 1.0E+04 | 7.3E+02 | 7% | 2 | 3.7 | 25.2 |
| Q | Q | 1.0E+04 | 2.7E+02 | 3% | 2 | 3.6 | 25.0 |
| F | S | 9.9E+03 | 3.6E+03 | 36% | 2 | 3.5 | 24.2 |
| V | K | 9.9E+03 | 6.0E+02 | 6% | 2 | 3.5 | 24.2 |
| A | V | 9.7E+03 | 3.4E+02 | 4% | 2 | 3.5 | 23.9 |
| W | P | 9.7E+03 | 1.4E+04 | 140% | 2 | 3.5 | 23.8 |
| Q | R | 9.6E+03 | 7.8E+02 | 8% | 2 | 3.4 | 23.7 |
| F | F | 9.5E+03 | 6.4E+02 | 7% | 2 | 3.4 | 23.4 |
| Y | R | 9.5E+03 | 3.1E+02 | 3% | 2 | 3.4 | 23.4 |
| F | L | 9.5E+03 | 9.5E+02 | 10% | 2 | 3.4 | 23.4 |
| P | K | 9.4E+03 | 2.1E+01 | 0% | 2 | 3.3 | 23.0 |
| A | S | 9.1E+03 | 7.9E+02 | 9% | 2 | 3.2 | 22.3 |
| A | M | 9.0E+03 | 5.6E+02 | 6% | 2 | 3.2 | 22.1 |
| P | F | 9.0E+03 | 2.5E+02 | 3% | 2 | 3.2 | 22.0 |
| F | A | 8.9E+03 | 7.0E+02 | 8% | 2 | 3.2 | 21.8 |
| I | A | 8.7E+03 | 1.9E+03 | 22% | 2 | 3.1 | 21.4 |
| Y | Q | 8.6E+03 | 3.2E+02 | 4% | 2 | 3.1 | 21.1 |
| I | G | 8.6E+03 | 3.8E+02 | 4% | 2 | 3.1 | 21.0 |
| V | F | 8.4E+03 | 8.1E+02 | 10% | 2 | 3.0 | 20.5 |
| M | N | 8.3E+03 | 5.3E+02 | 6% | 2 | 3.0 | 20.4 |
| Q | M | 8.1E+03 | 9.4E+02 | 12% | 2 | 2.9 | 19.9 |
| L | G | 8.1E+03 | 1.3E+02 | 2% | 2 | 2.9 | 19.9 |
| Q | I | 7.9E+03 | 2.5E+02 | 3% | 2 | 2.8 | 19.3 |
| Q | W | 7.8E+03 | 4.0E+02 | 5% | 2 | 2.8 | 19.0 |
| P | L | 7.7E+03 | 1.4E+01 | 0% | 2 | 2.7 | 18.8 |
| V | S | 7.6E+03 | 8.8E+02 | 12% | 2 | 2.7 | 18.7 |
| P | E | 7.5E+03 | 1.4E+02 | 2% | 2 | 2.7 | 18.5 |
| Y | T | 7.5E+03 | 3.5E+02 | 5% | 2 | 2.7 | 18.5 |
| Q | T | 7.4E+03 | 3.9E+02 | 5% | 2 | 2.6 | 18.1 |
| A | H | 7.4E+03 | 2.5E+02 | 3% | 2 | 2.6 | 18.1 |
| Q | H | 7.3E+03 | 2.0E+03 | 28% | 2 | 2.6 | 18.0 |
| P | G | 7.2E+03 | 1.4E+02 | 2% | 2 | 2.6 | 17.6 |
| W | M | 7.2E+03 | 1.9E+02 | 3% | 2 | 2.6 | 17.6 |
| F | V | 7.1E+03 | 2.4E+02 | 3% | 2 | 2.5 | 17.3 |
| P | A | 7.0E+03 | 6.7E+01 | 1% | 2 | 2.5 | 17.3 |
| F | I | 7.0E+03 | 8.1E+02 | 12% | 2 | 2.5 | 17.1 |
| M | E | 6.8E+03 | 4.8E+02 | 7% | 2 | 2.4 | 16.7 |
| P | N | 6.7E+03 | 6.7E+01 | 1% | 2 | 2.4 | 16.4 |
| Y | S | 6.5E+03 | 3.4E+02 | 5% | 2 | 2.3 | 15.9 |
| Y | W | 6.4E+03 | 1.3E+01 | 0% | 2 | 2.3 | 15.7 |
| A | L | 6.3E+03 | 5.9E+02 | 9% | 2 | 2.2 | 15.4 |
| A | A | 6.2E+03 | 3.2E+01 | 1% | 2 | 2.2 | 15.3 |
| F | N | 6.2E+03 | 8.0E+02 | 13% | 2 | 2.2 | 15.1 |
| A | F | 5.9E+03 | 4.0E+02 | 7% | 2 | 2.1 | 14.5 |
| M | G | 5.9E+03 | 4.7E+02 | 8% | 2 | 2.1 | 14.5 |
| F | E | 5.6E+03 | 2.8E+02 | 5% | 2 | 2.0 | 13.7 |
| Q | V | 5.4E+03 | 6.9E+02 | 13% | 2 | 1.9 | 13.4 |
| V | E | 5.4E+03 | 1.3E+02 | 2% | 2 | 1.9 | 13.2 |
| W | H | 5.4E+03 | 2.8E+02 | 5% | 2 | 1.9 | 13.1 |
| Q | K | 5.3E+03 | 6.4E+02 | 12% | 2 | 1.9 | 13.0 |
| V | N | 5.2E+03 | 3.5E+02 | 7% | 2 | 1.8 | 12.7 |
| V | G | 5.1E+03 | 3.0E+02 | 6% | 2 | 1.8 | 12.5 |
| V | L | 5.0E+03 | 1.9E+02 | 4% | 2 | 1.8 | 12.3 |
| Y | K | 5.0E+03 | 1.2E+02 | 2% | 2 | 1.8 | 12.3 |
| Y | H | 4.8E+03 | 3.6E+02 | 7% | 2 | 1.7 | 11.9 |
| Y | A | 4.8E+03 | 2.4E+02 | 5% | 2 | 1.7 | 11.7 |
| Q | Y | 4.7E+03 | 1.8E+02 | 4% | 2 | 1.7 | 11.5 |
| W | Y | 4.3E+03 | 5.3E+01 | 1% | 2 | 1.5 | 10.7 |
| Y | M | 4.3E+03 | 6.3E+01 | 1% | 2 | 1.5 | 10.4 |
| V | A | 4.2E+03 | 5.2E+02 | 12% | 2 | 1.5 | 10.3 |
| A | N | 4.0E+03 | 2.4E+02 | 6% | 2 | 1.4 | 9.8 |
| W | V | 3.9E+03 | 3.7E+02 | 9% | 2 | 1.4 | 9.7 |
| Y | Y | 3.8E+03 | 1.8E+02 | 5% | 2 | 1.4 | 9.3 |
| W | W | 3.8E+03 | 6.0E+02 | 16% | 2 | 1.3 | 9.2 |
| Y | G | 3.7E+03 | 2.8E+01 | 1% | 2 | 1.3 | 9.0 |
| Q | F | 3.6E+03 | 2.2E+01 | 1% | 2 | 1.3 | 8.9 |
| R | D | 3.6E+03 | 1.8E+03 | 50% | 2 | 1.3 | 8.8 |
| Q | L | 3.5E+03 | 2.3E+01 | 1% | 2 | 1.2 | 8.5 |
| A | G | 3.4E+03 | 1.4E+01 | 0% | 2 | 1.2 | 8.3 |
| A | E | 3.3E+03 | 5.8E+01 | 2% | 2 | 1.2 | 8.2 |
| W | N | 3.2E+03 | 2.2E+02 | 7% | 2 | 1.1 | 7.9 |
| L | D | 3.1E+03 | 4.2E+02 | 13% | 2 | 1.1 | 7.7 |
| Q | S | 3.1E+03 | 2.1E+02 | 7% | 2 | 1.1 | 7.6 |
| L | D | 3.1E+03 | 2.5E+02 | 8% | 2 | 1.1 | 7.5 |
| L | D | 3.0E+03 | 4.1E+02 | 13% | 2 | 1.1 | 7.4 |
| L | D | 3.0E+03 | 4.0E+02 | 13% | 2 | 1.1 | 7.3 |
| L | D | 3.0E+03 | 1.4E+02 | 5% | 2 | 1.1 | 7.3 |
| L | D | 3.0E+03 | 1.9E+02 | 6% | 2 | 1.1 | 7.3 |
| G | W | 2.9E+03 | 1.4E+02 | 5% | 2 | 1.0 | 7.2 |
| L | D | 2.8E+03 | 3.7E+02 | 13% | 2 | 1.0 | 7.0 |
| H | W | 2.8E+03 | 8.3E+01 | 3% | 2 | 1.0 | 6.9 |
| R | P | 2.8E+03 | 4.5E+02 | 16% | 2 | 1.0 | 6.9 |
| G | T | 2.7E+03 | 5.4E+01 | 2% | 2 | 1.0 | 6.7 |
| L | D | 2.7E+03 | 1.1E+01 | 0% | 2 | 1.0 | 6.7 |
| T | W | 2.7E+03 | 2.4E+02 | 9% | 2 | 1.0 | 6.7 |
| L | D | 2.7E+03 | 8.3E+00 | 0% | 2 | 1.0 | 6.6 |
| L | D | 2.7E+03 | 2.1E+02 | 8% | 2 | 1.0 | 6.6 |
| G | R | 2.7E+03 | 2.1E+01 | 1% | 2 | 1.0 | 6.6 |
| L | D | 2.7E+03 | 1.5E+02 | 6% | 2 | 1.0 | 6.6 |
| Y | F | 2.6E+03 | 1.1E+02 | 4% | 2 | 0.9 | 6.5 |
| L | D | 2.6E+03 | 1.8E+02 | 7% | 2 | 0.9 | 6.3 |
| L | D | 2.5E+03 | 1.3E+02 | 5% | 2 | 0.9 | 6.1 |
| W | F | 2.4E+03 | 2.3E+00 | 0% | 2 | 0.9 | 5.9 |
| T | R | 2.3E+03 | 8.8E+00 | 0% | 2 | 0.8 | 5.6 |
| S | R | 2.3E+03 | 2.0E+00 | 0% | 2 | 0.8 | 5.6 |
| Y | N | 2.2E+03 | 8.4E+01 | 4% | 2 | 0.8 | 5.4 |
| H | R | 2.2E+03 | 3.2E+01 | 2% | 2 | 0.8 | 5.3 |
| W | E | 2.1E+03 | 3.7E+02 | 17% | 2 | 0.7 | 5.2 |
| Y | L | 2.1E+03 | 1.3E+02 | 6% | 2 | 0.7 | 5.1 |
| A | D | 2.1E+03 | 1.7E+03 | 80% | 2 | 0.7 | 5.1 |
| Y | I | 2.1E+03 | 5.1E+01 | 2% | 2 | 0.7 | 5.1 |
| I | D | 2.0E+03 | 1.3E+02 | 7% | 2 | 0.7 | 4.9 |
| H | H | 2.0E+03 | 1.3E+02 | 7% | 2 | 0.7 | 4.8 |
| P | D | 2.0E+03 | 1.3E+01 | 1% | 2 | 0.7 | 4.8 |
| Q | N | 2.0E+03 | 5.7E+01 | 3% | 2 | 0.7 | 4.8 |
| W | I | 1.9E+03 | 1.6E−02 | 0% | 2 | 0.7 | 4.6 |
| Q | A | 1.8E+03 | 1.4E+01 | 1% | 2 | 0.7 | 4.5 |
| Q | E | 1.8E+03 | 1.6E+02 | 9% | 2 | 0.7 | 4.5 |
| W | L | 1.8E+03 | 3.0E+02 | 17% | 2 | 0.6 | 4.4 |
| H | Q | 1.8E+03 | 2.5E+02 | 14% | 2 | 0.6 | 4.3 |
| Y | V | 1.7E+03 | 3.0E+01 | 2% | 2 | 0.6 | 4.3 |
| Y | E | 1.7E+03 | 2.7E+01 | 2% | 2 | 0.6 | 4.1 |
| K | D | 1.6E+03 | 1.0E+02 | 6% | 2 | 0.6 | 4.0 |
| M | D | 1.6E+03 | 1.6E+02 | 10% | 2 | 0.6 | 4.0 |
| S | W | 1.5E+03 | 1.3E+02 | 9% | 2 | 0.5 | 3.8 |
| H | T | 1.5E+03 | 3.7E+01 | 2% | 2 | 0.5 | 3.7 |
| A | P | 1.5E+03 | 6.0E+00 | 0% | 2 | 0.5 | 3.6 |
| G | Y | 1.5E+03 | 9.3E+00 | 1% | 2 | 0.5 | 3.6 |
| R | W | 1.5E+03 | 2.0E+02 | 14% | 2 | 0.5 | 3.6 |
| N | R | 1.4E+03 | 8.6E+01 | 6% | 2 | 0.5 | 3.5 |
| S | T | 1.4E+03 | 3.3E+00 | 0% | 2 | 0.5 | 3.5 |
| T | Q | 1.4E+03 | 2.0E+01 | 1% | 2 | 0.5 | 3.4 |
| T | Y | 1.4E+03 | 9.7E+00 | 1% | 2 | 0.5 | 3.4 |
| G | Q | 1.4E+03 | 7.7E+00 | 1% | 2 | 0.5 | 3.4 |
| I | P | 1.3E+03 | 9.9E+01 | 7% | 2 | 0.5 | 3.3 |
| T | H | 1.3E+03 | 9.5E+01 | 7% | 2 | 0.5 | 3.2 |
| G | S | 1.3E+03 | 1.6E+00 | 0% | 2 | 0.5 | 3.2 |
| F | D | 1.3E+03 | 2.6E+01 | 2% | 2 | 0.5 | 3.2 |
| G | I | 1.3E+03 | 1.2E−01 | 0% | 2 | 0.5 | 3.1 |
| H | M | 1.3E+03 | 2.1E+02 | 17% | 2 | 0.5 | 3.1 |
| T | T | 1.3E+03 | 2.7E+00 | 0% | 2 | 0.5 | 3.1 |
| H | Y | 1.3E+03 | 5.6E+01 | 4% | 2 | 0.5 | 3.1 |
| H | S | 1.3E+03 | 2.2E+01 | 2% | 2 | 0.5 | 3.1 |
| T | M | 1.2E+03 | 3.2E+01 | 3% | 2 | 0.4 | 3.1 |
| G | K | 1.2E+03 | 2.0E+01 | 2% | 2 | 0.4 | 3.0 |
| G | H | 1.2E+03 | 5.5E+01 | 5% | 2 | 0.4 | 3.0 |
| G | M | 1.2E+03 | 2.4E+01 | 2% | 2 | 0.4 | 2.9 |
| S | Q | 1.2E+03 | 2.4E+01 | 2% | 2 | 0.4 | 2.9 |
| H | K | 1.2E+03 | 2.1E+01 | 2% | 2 | 0.4 | 2.9 |
| E | D | 1.2E+03 | 1.7E+03 | 141% | 2 | 0.4 | 2.9 |
| T | I | 1.2E+03 | 4.4E+01 | 4% | 2 | 0.4 | 2.9 |
| G | F | 1.2E+03 | 3.9E+01 | 3% | 2 | 0.4 | 2.9 |
| W | D | 1.2E+03 | 4.3E+01 | 4% | 2 | 0.4 | 2.9 |
| N | W | 1.2E+03 | 1.8E+02 | 15% | 2 | 0.4 | 2.8 |
| G | V | 1.2E+03 | 2.0E+01 | 2% | 2 | 0.4 | 2.8 |
| T | K | 1.1E+03 | 1.5E+01 | 1% | 2 | 0.4 | 2.8 |
| K | W | 1.1E+03 | 3.4E+01 | 3% | 2 | 0.4 | 2.8 |
| E | R | 1.1E+03 | 2.5E+01 | 2% | 2 | 0.4 | 2.7 |
| V | D | 1.1E+03 | 8.2E+01 | 8% | 2 | 0.4 | 2.7 |
| S | K | 1.1E+03 | 1.0E+01 | 1% | 2 | 0.4 | 2.7 |
| S | Y | 1.1E+03 | 1.0E+02 | 9% | 2 | 0.4 | 2.6 |
| S | I | 1.1E+03 | 4.6E+00 | 0% | 2 | 0.4 | 2.6 |
| Q | G | 1.1E+03 | 9.4E+01 | 9% | 2 | 0.4 | 2.6 |
| G | L | 1.1E+03 | 2.8E+01 | 3% | 2 | 0.4 | 2.6 |
| S | H | 1.1E+03 | 2.6E+01 | 3% | 2 | 0.4 | 2.6 |
| S | S | 1.0E+03 | 1.6E+01 | 2% | 2 | 0.4 | 2.5 |
| H | F | 1.0E+03 | 3.1E+02 | 30% | 2 | 0.4 | 2.5 |
| T | D | 1.0E+03 | 3.0E+01 | 3% | 2 | 0.4 | 2.5 |
| H | G | 1.0E+03 | 1.7E+02 | 17% | 2 | 0.4 | 2.5 |
| D | H | 1.0E+03 | 6.5E+02 | 65% | 2 | 0.4 | 2.5 |
| R | I | 1.0E+03 | 2.0E+01 | 2% | 2 | 0.4 | 2.4 |
| M | P | 9.9E+02 | 3.8E+02 | 39% | 2 | 0.4 | 2.4 |
| K | I | 9.9E+02 | 1.1E+01 | 1% | 2 | 0.4 | 2.4 |
| T | V | 9.8E+02 | 1.3E+01 | 1% | 2 | 0.3 | 2.4 |
| R | R | 9.8E+02 | 4.6E+01 | 5% | 2 | 0.3 | 2.4 |
| H | A | 9.6E+02 | 7.2E+01 | 7% | 2 | 0.3 | 2.4 |
| S | M | 9.6E+02 | 4.3E+00 | 0% | 2 | 0.3 | 2.4 |
| R | H | 9.6E+02 | 4.5E+01 | 5% | 2 | 0.3 | 2.3 |
| G | A | 9.5E+02 | 1.2E+01 | 1% | 2 | 0.3 | 2.3 |
| S | F | 9.4E+02 | 6.8E+01 | 7% | 2 | 0.3 | 2.3 |
| R | L | 9.2E+02 | 1.9E+01 | 2% | 2 | 0.3 | 2.3 |
| K | H | 9.0E+02 | 2.6E+02 | 29% | 2 | 0.3 | 2.2 |
| T | S | 8.9E+02 | 2.5E+01 | 3% | 2 | 0.3 | 2.2 |
| S | V | 8.9E+02 | 2.1E+01 | 2% | 2 | 0.3 | 2.2 |
| Y | D | 8.9E+02 | 1.1E+01 | 1% | 2 | 0.3 | 2.2 |
| R | G | 8.8E+02 | 3.9E+02 | 44% | 2 | 0.3 | 2.2 |
| K | L | 8.8E+02 | 4.9E+01 | 6% | 2 | 0.3 | 2.2 |
| N | T | 8.7E+02 | 2.4E+01 | 3% | 2 | 0.3 | 2.1 |
| R | E | 8.7E+02 | 1.1E+02 | 12% | 2 | 0.3 | 2.1 |
| E | F | 8.7E+02 | 2.2E+02 | 25% | 2 | 0.3 | 2.1 |
| P | P | 8.5E+02 | 3.5E+01 | 4% | 2 | 0.3 | 2.1 |
| G | G | 8.2E+02 | 1.7E+01 | 2% | 2 | 0.3 | 2.0 |
| T | P | 8.2E+02 | 3.8E+01 | 5% | 2 | 0.3 | 2.0 |
| H | I | 8.2E+02 | 3.6E+02 | 44% | 2 | 0.3 | 2.0 |
| H | N | 8.0E+02 | 1.6E+01 | 2% | 2 | 0.3 | 2.0 |
| K | M | 8.0E+02 | 1.4E+01 | 2% | 2 | 0.3 | 2.0 |
| S | L | 7.9E+02 | 5.5E+01 | 7% | 2 | 0.3 | 1.9 |
| R | M | 7.9E+02 | 4.1E+01 | 5% | 2 | 0.3 | 1.9 |
| S | A | 7.9E+02 | 4.4E+01 | 6% | 2 | 0.3 | 1.9 |
| N | Y | 7.8E+02 | 3.6E+01 | 5% | 2 | 0.3 | 1.9 |
| N | F | 7.8E+02 | 1.3E+02 | 16% | 2 | 0.3 | 1.9 |
| K | R | 7.8E+02 | 1.2E+01 | 2% | 2 | 0.3 | 1.9 |
| S | G | 7.7E+02 | 3.3E+01 | 4% | 2 | 0.3 | 1.9 |
| T | G | 7.7E+02 | 1.3E+02 | 17% | 2 | 0.3 | 1.9 |
| R | F | 7.7E+02 | 5.3E+01 | 7% | 2 | 0.3 | 1.9 |
| K | F | 7.5E+02 | 2.4E+02 | 32% | 2 | 0.3 | 1.8 |
| N | K | 7.5E+02 | 8.5E+00 | 1% | 2 | 0.3 | 1.8 |
| T | L | 7.5E+02 | 4.7E+01 | 6% | 2 | 0.3 | 1.8 |
| N | Q | 7.4E+02 | 9.6E+01 | 13% | 2 | 0.3 | 1.8 |
| H | P | 7.4E+02 | 3.1E+02 | 41% | 2 | 0.3 | 1.8 |
| S | D | 7.4E+02 | 2.0E+02 | 27% | 2 | 0.3 | 1.8 |
| F | P | 7.3E+02 | 4.6E+02 | 63% | 2 | 0.3 | 1.8 |
| N | M | 7.2E+02 | 5.1E+01 | 7% | 2 | 0.3 | 1.8 |
| K | Y | 7.0E+02 | 3.6E+01 | 5% | 2 | 0.2 | 1.7 |
| G | N | 7.0E+02 | 3.8E+01 | 5% | 2 | 0.2 | 1.7 |
| T | E | 7.0E+02 | 5.0E+01 | 7% | 2 | 0.2 | 1.7 |
| R | Y | 6.9E+02 | 2.1E+00 | 0% | 2 | 0.2 | 1.7 |
| E | H | 6.9E+02 | 2.1E+02 | 31% | 2 | 0.2 | 1.7 |
| R | A | 6.7E+02 | 5.7E+01 | 9% | 2 | 0.2 | 1.6 |
| R | N | 6.7E+02 | 3.7E+01 | 6% | 2 | 0.2 | 1.6 |
| R | S | 6.6E+02 | 3.9E+00 | 1% | 2 | 0.2 | 1.6 |
| N | H | 6.6E+02 | 2.9E+00 | 0% | 2 | 0.2 | 1.6 |
| E | K | 6.5E+02 | 2.3E+02 | 35% | 2 | 0.2 | 1.6 |
| N | L | 6.5E+02 | 1.8E+02 | 27% | 2 | 0.2 | 1.6 |
| N | I | 6.4E+02 | 1.0E+02 | 16% | 2 | 0.2 | 1.6 |
| R | Q | 6.4E+02 | 3.2E+01 | 5% | 2 | 0.2 | 1.6 |
| T | F | 6.4E+02 | 5.3E+02 | 83% | 2 | 0.2 | 1.6 |
| R | T | 6.4E+02 | 1.6E+02 | 25% | 2 | 0.2 | 1.6 |
| N | S | 6.4E+02 | 6.0E+01 | 9% | 2 | 0.2 | 1.6 |
| K | Q | 6.4E+02 | 2.1E+01 | 3% | 2 | 0.2 | 1.6 |
| T | A | 6.3E+02 | 3.2E+00 | 1% | 2 | 0.2 | 1.6 |
| K | T | 6.1E+02 | 2.3E+00 | 0% | 2 | 0.2 | 1.5 |
| D | F | 6.0E+02 | 8.5E+02 | 141% | 2 | 0.2 | 1.5 |
| T | N | 6.0E+02 | 1.9E+01 | 3% | 2 | 0.2 | 1.5 |
| R | K | 5.9E+02 | 1.1E+01 | 2% | 2 | 0.2 | 1.5 |
| K | V | 5.9E+02 | 2.6E+01 | 4% | 2 | 0.2 | 1.5 |
| R | V | 5.9E+02 | 1.9E+01 | 3% | 2 | 0.2 | 1.4 |
| N | V | 5.5E+02 | 5.8E+01 | 11% | 2 | 0.2 | 1.4 |
| S | N | 5.4E+02 | 1.7E+01 | 3% | 2 | 0.2 | 1.3 |
| G | D | 5.4E+02 | 9.1E+01 | 17% | 2 | 0.2 | 1.3 |
| H | V | 5.4E+02 | 5.2E+02 | 97% | 2 | 0.2 | 1.3 |
| K | K | 5.2E+02 | 2.9E+01 | 6% | 2 | 0.2 | 1.3 |
| G | E | 5.1E+02 | 1.5E+01 | 3% | 2 | 0.2 | 1.3 |
| N | G | 5.0E+02 | 8.1E+01 | 16% | 2 | 0.2 | 1.2 |
| Q | D | 5.0E+02 | 7.9E+01 | 16% | 2 | 0.2 | 1.2 |
| H | D | 4.9E+02 | 9.0E+01 | 19% | 2 | 0.2 | 1.2 |
| H | L | 4.7E+02 | 8.8E+00 | 2% | 2 | 0.2 | 1.1 |
| K | S | 4.7E+02 | 1.2E+01 | 3% | 2 | 0.2 | 1.1 |
| E | I | 4.6E+02 | 1.6E+02 | 36% | 2 | 0.2 | 1.1 |
| D | V | 4.6E+02 | 6.4E+02 | 140% | 2 | 0.2 | 1.1 |
| K | G | 4.6E+02 | 1.2E+01 | 3% | 2 | 0.2 | 1.1 |
| S | P | 4.5E+02 | 3.6E+01 | 8% | 2 | 0.2 | 1.1 |
| N | A | 4.3E+02 | 1.1E+02 | 27% | 2 | 0.2 | 1.0 |
| G | P | 4.3E+02 | 2.4E+00 | 1% | 2 | 0.2 | 1.0 |
| E | Y | 4.3E+02 | 2.1E+02 | 50% | 2 | 0.2 | 1.0 |
| D | Y | 4.1E+02 | 5.8E+02 | 140% | 2 | 0.1 | 1.0 |
| E | A | 4.0E+02 | 2.1E+02 | 53% | 2 | 0.1 | 1.0 |
| S | E | 3.9E+02 | 9.2E+00 | 2% | 2 | 0.1 | 1.0 |
| E | W | 3.9E+02 | 3.9E+02 | 101% | 2 | 0.1 | 0.9 |
| E | T | 3.9E+02 | 1.0E+02 | 27% | 2 | 0.1 | 0.9 |
| Y | P | 3.8E+02 | 8.1E+01 | 21% | 2 | 0.1 | 0.9 |
| K | N | 3.7E+02 | 2.6E+01 | 7% | 2 | 0.1 | 0.9 |
| K | A | 3.6E+02 | 5.9E+00 | 2% | 2 | 0.1 | 0.9 |
| D | G | 3.5E+02 | 2.7E+02 | 78% | 2 | 0.1 | 0.8 |
| D | R | 3.4E+02 | 1.8E+02 | 54% | 2 | 0.1 | 0.8 |
| N | N | 2.9E+02 | 1.6E+02 | 55% | 2 | 0.1 | 0.7 |
| K | E | 2.7E+02 | 2.1E+02 | 77% | 2 | 0.1 | 0.7 |
| D | T | 2.5E+02 | 3.4E+02 | 140% | 2 | 0.1 | 0.6 |
| H | E | 2.3E+02 | 2.3E+02 | 98% | 2 | 0.1 | 0.6 |
| D | L | 2.1E+02 | 2.8E+02 | 137% | 2 | 0.1 | 0.5 |
| E | L | 2.1E+02 | 2.2E+02 | 107% | 2 | 0.1 | 0.5 |
| E | V | 2.0E+02 | 2.0E+02 | 98% | 2 | 0.1 | 0.5 |
| D | Q | 1.6E+02 | 2.3E+02 | 140% | 2 | 0.1 | 0.4 |
| E | S | 1.6E+02 | 1.2E+02 | 75% | 2 | 0.1 | 0.4 |
| E | M | 1.5E+02 | 1.4E+02 | 89% | 2 | 0.1 | 0.4 |
| N | E | 1.5E+02 | 2.1E+02 | 137% | 2 | 0.1 | 0.4 |
| D | M | 1.4E+02 | 1.9E+02 | 137% | 2 | 0.05 | 0.3 |
| D | N | 1.3E+02 | 1.8E+02 | 141% | 2 | 0.05 | 0.3 |
| E | Q | 1.1E+02 | 8.4E+01 | 77% | 2 | 0.04 | 0.3 |
| E | G | 9.8E+01 | 6.1E+01 | 62% | 2 | 0.03 | 0.2 |
| V | P | 9.5E+01 | 8.2E+01 | 86% | 2 | 0.03 | 0.2 |
| D | I | 3.0E+01 | 3.0E+01 | 101% | 2 | 0.01 | 0.1 |
| E | N | 1.7E+01 | 2.1E+01 | 121% | 2 | 0.01 | 0.04 |
| D | S | 1.5E+01 | 1.8E+01 | 126% | 2 | 0.01 | 0.04 |
| E | E | 1.4E+01 | 1.8E+01 | 126% | 2 | 0.01 | 0.03 |
| D | W | 1.4E+01 | 1.5E+01 | 108% | 2 | 0.01 | 0.03 |
| L | P | 1.1E+01 | 3.2E+00 | 29% | 2 | 0.004 | 0.03 |
| D | K | 1.0E+01 | 1.3E+01 | 121% | 2 | 0.004 | 0.03 |
| N | P | 9.0E+00 | 1.2E+01 | 134% | 2 | 0.003 | 0.02 |
| D | A | 7.7E+00 | 8.8E+00 | 114% | 2 | 0.003 | 0.02 |
| N | D | 3.0E+00 | 3.7E+00 | 123% | 2 | 0.001 | 0.01 |
| E | P | 2.0E+00 | 2.9E+00 | 141% | 2 | 0.001 | 0.005 |
| D | D | 2.2E−03 | 2.2E−03 | 101% | 2 | 0.000 | 0.000 |
| D | P | 6.6E−04 | 1.3E−04 | 19% | 2 | 0.000 | 0.000 |
| K | P | 5.6E−04 | 9.2E−06 | 2% | 2 | 0.000 | 0.000 |
| D | E | 4.0E−04 | 5.6E−04 | 141% | 2 | 0.000 | 0.000 |
| Q | P | 0.0E+00 | 0.0E+00 | n/a | 2 | 0.000 | 0.000 |

TABLE 3
Rationally Designed (Natural Sequence) Quenched Fluorescence Library

| | | | | | | | | | | | | | | Fold- | Fold- |
|---|---|---|---|---|---|---|---|---|---|---|---|---|---|---|---|
| | | | | | | | | | | | k_{cat}/K_{M} | | | Improved | Improved |
| Natural Substrate | X₁₀ | X₉ | X₈ | X₇ | X₆ | X₅ | X₄ | X₃ | X₂ | X₁ | (M⁻¹s⁻¹) | % CV | n = | (PAR-1) | (FpA) |
| FpA_P | D | F | L | A | E | G | G | G | P | R | 4.0E+04 | 23% | 18 | 14.2 | 97.8 |
| Factor V (1) | H | T | H | H | A | P | L | S | P | R | 3.1E+04 | 39% | 12 | 11.2 | 77.2 |
| Thrombin (1) | D | Q | V | T | V | A | M | T | P | R | 3.0E+04 | 11% | 12 | 10.7 | 73.6 |
| Factor XI | N | E | S | T | T | K | I | K | P | R | 2.0E+04 | 11% | 18 | 7.1 | 49.2 |
| Thrombin (2) | S | E | Y | Q | T | F | F | N | P | R | 2.0E+04 | 16% | 6 | 7.1 | 48.7 |
| Factor VIII (1) | L | S | K | N | N | A | I | E | P | R | 1.3E+04 | 6% | 6 | 4.7 | 32.3 |
| Factor XIII | T | V | E | L | Q | G | V | V | P | R | 1.2E+04 | 42% | 11 | 4.2 | 28.7 |
| PAR 4 | S | T | P | S | I | L | P | A | P | R | 9.4E+03 | 18% | 11 | 3.4 | 23.1 |
| TAFI | Q | I | S | N | D | T | V | S | P | R | 5.3E+03 | 26% | 6 | 1.9 | 13.1 |
| PAR 1 | K | A | T | N | A | T | L | D | P | R | 3.9E+03 | 10% | 18 | 1.4 | 9.7 |
| PAR 1 | — | — | T | N | A | T | L | D | P | R | 3.6E+03 | 16% | 11 | 1.3 | 8.9 |
| PAR 1 | — | — | — | — | A | T | L | D | P | R | 3.3E+03 | 15% | 12 | 1.2 | 8.2 |
| Protein C | E | D | Q | E | D | Q | V | D | P | R | 1.8E+03 | 5% | 6 | 0.6 | 4.4 |
| Factor VIII (2) | Y | D | E | D | E | N | Q | S | P | R | 9.3E+02 | 50% | 12 | 0.3 | 2.3 |
| Factor V (2) | N | R | L | A | A | A | L | G | I | R | 7.2E+02 | 137% | 6 | 0.3 | 1.8 |
| Osteopontin | R | G | D | S | V | V | Y | G | L | R | 4.1E+02 | 113% | 6 | 0.1 | 1.0 |
| FpA | D | F | L | A | E | G | G | G | V | R | 4.1E+02 | 79% | 9 | 0.1 | 1.0 |
| FpB | D | N | E | E | G | F | F | S | A | R | 3.0E+02 | 48% | 6 | 0.1 | 0.7 |
| Factor V (3) | P | D | N | I | A | A | W | Y | L | R | 9.8E+01 | 15% | 6 | 0.03 | 0.2 |
| PAR 3 | N | L | A | K | P | T | L | P | I | K | 1.6E+01 | 73% | 5 | 0.01 | 0.04 |
| Antithrombin | A | S | T | A | V | V | I | A | G | R | 3.1E−01 | 75% | 6 | 0.000 | 0.001 |
| Thrombin (3) | E | D | S | D | R | A | I | E | G | R | 0.0E+00 | n/a | 7 | 0.000 | 0.000 |

### Example 4

### Screening the Quenched Fluorescence Peptide Library to Evaluate the Kinetic Rate Constant for Factor Xa Cleavage and Identify Thrombin-Specific Cleavage Sequences

The objective was to identify the preferred thrombin cleavage sequences described herein with respect to Example 3, above, that additionally display the lowest rates for cleavage by Factor Xa. In order to determine the cleavage rates for the QF peptide substrate library, a progress curve protocol was designed for evaluating the kinetics of substrate cleavage by Factor Xa relative to that of thrombin. The protocol was essentially as described above for α-thrombin with only minor modifications. The progress curve method assumed that the reaction followed a simple Michaelis Menten mechanism with the encounter complex of substrate and enzyme being limiting (i.e. psuedo-1^{St}-order). Under conditions where [QF-substrate]<<K_{M} this method allowed for an estimation of the k_{cat}/K_{M} from an exponential fit of the complete reaction progress curves (i.e. complete substrate hydrolysis over time). The stock concentrations of each QF substrate (up to 96 per plate) were confirmed to be in the range of ˜3500-4500 μM using the plate method described in Example 2 above. To initiate the cleavage reactions, the QF peptide substrates (in a 96-well format) were first diluted to ˜500 μM in 100% DMSO by mixing 10 μL of stock substrate+80 μL DMSO followed by two subsequent serial dilutions with assay buffer (50 mM Hepes, 150 mM NaCl, 10 mM CaCl₂, 0.1% PEG8000, pH 7.4) taking 20 μl of dilution 1+80 μL assay buffer (˜100 μM in 20% DMSO) and then 20 μL of dilution 2+180 μL assay buffer (˜10 μM in 2% DMSO). Human plasma purified Factor Xa (Molecular Innovations, Inc, Novi Mich., USA) was diluted from the stock to a working concentration of 4 μM in assay buffer. Progress curve reactions were initiated by combining 100 μL of QF substrate dilution three (˜10 μM in 2% DMSO) with 80 μL of assay buffer and 20 μL of 4 μM thrombin in a 96-well black assay plate. Reactions were followed in a Molecular Devices fluorescence spectrometer for 3 hours at 37° C. using an excitation wavelength of 320 nm and an emission wavelength of 420 nm without any cut-off filter. Data collected using the SoftMax Pro software were exported as .txt files for analysis using Excel analysis templates and non-linear regression analysis using the GraphPad/Prism software suite. Progress curves were fit to the following equation:

Y=F₀+Fₘₐₓ*(1−exp(−E*k*x))

where x=reaction time, F₀=the initial fluorescence intensity, Fₘₐₓ=the maximum fluorescence intensity at complete hydrolysis, k=the kinetic rate constant in the form of k_{cat}/K_{M} with the units of M⁻¹s⁻¹ and E=the enzyme concentration in M units.

Tables 4 and Table 5 set forth the data generated from screening the quenched fluorescence positional scanning library and a set of rationally designed quenched fluorescence substrates based on natural thrombin cleavage sequences, respectively. The quenched fluorescence positional scanning library (X₄/X₃) was based on the PAR 1 thrombin cleavage sequence (Table 4), however, using a fixed prime side sequence of IVGG, such that the library is of the form Lys(Dnp)-ATNATX₄X₃PRIVGG-Lys(Abz), where Lys(Dnp) and Lys(Abz) (SEQ ID NO: 238) are the fluorescence quencher and donor moieties, respectively. The rationally designed QF-substrates based on natural thrombin cleavage sequences (Table 5) were synthesized by Aurigene (Bangalore, India) and also contained a fixed prime side sequence of IVGG, such that the library is of the form Lys(Dnp)-X₁₀, X₉X₈X₇X₆X₅X₄X₃X₂X₁IVGG-Lys(Abz), where Lys(Dnp) and Lys(Abz) (SEQ ID NO: 239) are the fluorescence quencher and donor moieties, respectively. Data are presented in Tables 4 and 5 as the ranked functional selectivity calculation, wherein functional selectivity is defined as the k_{cat}/K_{M} value for α-thrombin (FIIa) cleavage multiplied by the specificity ratio (Ala k_{cat}/K_{M} divided by FXa k_{cat}/K_{M}). As described herein, the QF-substrate sequences with a high functional selectivity value are a representation of those sequences which have the highest rate of α-thrombin cleavage paired to the greatest specificity for cleavage by α-thrombin compared to Factor Xa. Also shown are the specificity ratio and k_{cat}/K_{M} values for Factor Xa cleavage and thrombin (FIIa) cleavage of the substrate library (data reproduced from Example 3) with standard deviation and % CV shown for the FXa cleavage data.

Five sequences were selected from the QF-substrate library with >400-fold specificity ratio (Table 4). Thus of the QF-substrate sequences provided herein, the sequences (X₄-X₁) of FTPR, FKPR, LKPR, WQPR and WPPR showed the highest specificity ratio. In addition to these five sequences, the sequences; MTPR WTPR and MKPR demonstrated a ˜300-fold specificity ratios, but also functional selectivity values of 7.0E+06 to 1.0E+07.

TABLE 4
X₄/X₃ Positional Scanning Quenched Fluorescence Library
X₄/X₃ Positional Scanning Quenched Fluorescence Library:
form Lys(Dnp)-ATNATX₄X₃PRIVGG-Lys(Abz), where Lys(Dnp)
and Lys(Abz) (SEQ ID NO 238) are the fluorescence quencher
and donor moieties, respectively. All amino acid variants
herein can form part of FX molecules according to the invention.

| | | | | | | | | Functional |
|---|---|---|---|---|---|---|---|---|
| | | FXa | | | | FIIa | Specificity | Selectivity |
| | | k_{cat}/K_{M} | | | | k_{cat}/K_{M} | Ratio | (Specificity*FIIa |
| X₄ | X₃ | (M⁻¹s⁻¹) | S.D. | % CV | n | (M⁻¹s⁻¹) | (FIIa/FXa) | k_{cat}/K_{M}) |
| F | T | 4.9E+01 | 1.5E+01 | 31% | 2 | 2.7E+04 | 549 | 1.5E+07 |
| L | K | 7.2E+01 | 1.1E+01 | 16% | 2 | 3.2E+04 | 438 | 1.4E+07 |
| F | K | 5.5E+01 | 4.8E+00 | 9% | 2 | 2.5E+04 | 459 | 1.2E+07 |
| M | T | 9.7E+01 | 1.6E+01 | 17% | 2 | 3.2E+04 | 332 | 1.1E+07 |
| W | Q | 3.7E+01 | 2.2E+01 | 60% | 2 | 1.9E+04 | 511 | 9.8E+06 |
| M | K | 7.2E+01 | 4.0E+00 | 6% | 2 | 2.3E+04 | 312 | 7.0E+06 |
| W | T | 4.6E+01 | 3.0E−01 | 1% | 2 | 1.6E+04 | 355 | 5.8E+06 |
| W | P | 1.7E+01 | 2.2E+00 | 13% | 2 | 9.7E+03 | 581 | 5.6E+06 |
| W | S | 5.2E+01 | 8.8E+00 | 17% | 2 | 1.6E+04 | 318 | 5.2E+06 |
| A | Y | 2.6E+01 | 3.7E+00 | 15% | 2 | 1.1E+04 | 448 | 5.1E+06 |
| I | K | 1.2E+02 | 3.4E+01 | 28% | 2 | 2.4E+04 | 197 | 4.7E+06 |
| F | R | 4.3E+02 | 2.8E+02 | 66% | 2 | 4.3E+04 | 101 | 4.4E+06 |
| W | K | 4.1E+01 | 1.8E+00 | 4% | 2 | 1.3E+04 | 317 | 4.2E+06 |
| W | A | 4.2E+01 | 5.7E+00 | 13% | 2 | 1.3E+04 | 314 | 4.2E+06 |
| A | K | 3.0E+01 | 5.6E+00 | 19% | 2 | 1.1E+04 | 360 | 3.8E+06 |
| V | T | 6.8E+01 | 9.8E+00 | 14% | 2 | 1.6E+04 | 234 | 3.7E+06 |
| L | T | 5.4E+02 | 5.4E+01 | 10% | 2 | 4.4E+04 | 82 | 3.6E+06 |
| F | Q | 2.6E+02 | 1.8E+02 | 70% | 2 | 3.0E+04 | 117 | 3.5E+06 |
| L | R | 6.1E+02 | 2.8E+01 | 5% | 2 | 4.5E+04 | 74 | 3.3E+06 |
| M | V | 1.0E+02 | 2.2E+01 | 22% | 2 | 1.7E+04 | 167 | 2.8E+06 |
| V | W | 1.9E+02 | 5.4E+00 | 3% | 2 | 2.3E+04 | 122 | 2.8E+06 |
| L | I | 4.2E+02 | 6.9E+01 | 16% | 2 | 3.4E+04 | 80 | 2.7E+06 |
| L | Y | 1.6E+02 | 1.2E+01 | 7% | 2 | 2.1E+04 | 128 | 2.7E+06 |
| V | K | 3.8E+01 | 1.7E+00 | 5% | 2 | 9.9E+03 | 261 | 2.6E+06 |
| V | H | 6.7E+01 | 4.0E+00 | 6% | 2 | 1.3E+04 | 193 | 2.5E+06 |
| L | V | 3.6E+02 | 2.9E+01 | 8% | 2 | 3.0E+04 | 83 | 2.4E+06 |
| M | R | 4.9E+02 | 3.4E+01 | 7% | 2 | 3.5E+04 | 70 | 2.4E+06 |
| P | Y | 8.3E+01 | 1.0E+00 | 1% | 2 | 1.4E+04 | 168 | 2.3E+06 |
| L | F | 9.9E+01 | 9.8E+00 | 10% | 2 | 1.5E+04 | 151 | 2.3E+06 |
| A | V | 4.2E+01 | 5.4E+00 | 13% | 2 | 9.7E+03 | 230 | 2.2E+06 |
| V | V | 5.4E+01 | 5.4E+00 | 10% | 2 | 1.1E+04 | 203 | 2.2E+06 |
| W | G | 6.1E+01 | 1.3E+01 | 22% | 2 | 1.1E+04 | 188 | 2.1E+06 |
| M | F | 5.5E+01 | 9.6E+00 | 17% | 2 | 1.1E+04 | 195 | 2.1E+06 |
| V | Y | 6.1E+01 | 1.8E+00 | 3% | 2 | 1.1E+04 | 181 | 2.0E+06 |
| Y | T | 2.9E+01 | 5.3E+00 | 18% | 2 | 7.5E+03 | 263 | 2.0E+06 |
| L | Q | 1.2E+03 | 7.5E+01 | 6% | 2 | 4.9E+04 | 41 | 2.0E+06 |
| F | H | 1.5E+02 | 2.2E+01 | 15% | 2 | 1.7E+04 | 115 | 2.0E+06 |
| F | Y | 7.1E+01 | 8.3E+00 | 12% | 2 | 1.2E+04 | 164 | 1.9E+06 |
| F | F | 4.8E+01 | 1.4E+01 | 29% | 2 | 9.5E+03 | 200 | 1.9E+06 |
| F | G | 1.0E+02 | 1.0E+02 | 102% | 2 | 1.4E+04 | 134 | 1.8E+06 |
| I | R | 7.6E+02 | 4.0E+01 | 5% | 2 | 3.7E+04 | 49 | 1.8E+06 |
| V | Q | 2.5E+02 | 8.1E+00 | 3% | 2 | 2.1E+04 | 83 | 1.7E+06 |
| A | F | 2.2E+01 | 3.4E+00 | 15% | 2 | 5.9E+03 | 264 | 1.6E+06 |
| I | V | 3.6E+02 | 1.3E+02 | 37% | 2 | 2.4E+04 | 66 | 1.6E+06 |
| I | Y | 2.2E+02 | 1.6E+01 | 7% | 2 | 1.8E+04 | 81 | 1.5E+06 |
| M | I | 2.2E+02 | 2.6E+01 | 12% | 2 | 1.8E+04 | 81 | 1.4E+06 |
| V | I | 1.2E+02 | 2.9E+00 | 2% | 2 | 1.3E+04 | 108 | 1.4E+06 |
| I | I | 4.9E+02 | 7.1E+01 | 14% | 2 | 2.6E+04 | 53 | 1.4E+06 |
| L | W | 1.0E+03 | 1.2E+02 | 11% | 2 | 3.6E+04 | 36 | 1.3E+06 |
| I | F | 1.3E+02 | 2.8E+01 | 21% | 2 | 1.3E+04 | 99 | 1.3E+06 |
| V | F | 5.4E+01 | 7.3E+00 | 14% | 2 | 8.4E+03 | 155 | 1.3E+06 |
| M | Y | 1.1E+02 | 4.4E+01 | 39% | 2 | 1.2E+04 | 106 | 1.3E+06 |
| Y | Q | 6.0E+01 | 1.5E+01 | 26% | 2 | 8.6E+03 | 144 | 1.2E+06 |
| I | T | 8.4E+02 | 3.2E+01 | 4% | 2 | 3.2E+04 | 38 | 1.2E+06 |
| P | K | 7.4E+01 | 1.3E+01 | 17% | 2 | 9.4E+03 | 126 | 1.2E+06 |
| L | H | 5.4E+02 | 5.8E+01 | 11% | 2 | 2.5E+04 | 47 | 1.2E+06 |
| I | Q | 1.7E+03 | 1.9E+02 | 12% | 2 | 4.4E+04 | 27 | 1.2E+06 |
| P | F | 6.9E+01 | 6.2E+00 | 9% | 2 | 9.0E+03 | 129 | 1.2E+06 |
| R | P | 7.0E+00 | 2.5E+00 | 36% | 2 | 2.8E+03 | 405 | 1.1E+06 |
| M | Q | 9.5E+02 | 1.8E+01 | 2% | 2 | 3.3E+04 | 34 | 1.1E+06 |
| M | H | 2.9E+02 | 8.2E+01 | 28% | 2 | 1.8E+04 | 62 | 1.1E+06 |
| A | R | 3.5E+02 | 5.5E+01 | 16% | 2 | 2.0E+04 | 56 | 1.1E+06 |
| F | W | 3.7E+02 | 1.3E+02 | 35% | 2 | 2.0E+04 | 54 | 1.1E+06 |
| Y | S | 4.1E+01 | 2.3E+00 | 6% | 2 | 6.5E+03 | 158 | 1.0E+06 |
| Y | K | 2.5E+01 | 9.2E+00 | 37% | 2 | 5.0E+03 | 199 | 9.9E+05 |
| A | A | 4.5E+01 | 7.4E−01 | 2% | 2 | 6.2E+03 | 138 | 8.6E+05 |
| I | H | 8.1E+02 | 1.9E+01 | 2% | 2 | 2.6E+04 | 32 | 8.3E+05 |
| I | M | 2.1E+03 | 5.4E+02 | 26% | 2 | 4.1E+04 | 20 | 8.2E+05 |
| L | M | 2.7E+03 | 2.4E+02 | 9% | 2 | 4.7E+04 | 17 | 8.1E+05 |
| A | G | 1.4E+01 | 2.0E+00 | 14% | 2 | 3.4E+03 | 238 | 8.1E+05 |
| V | M | 2.8E+02 | 2.1E+01 | 8% | 2 | 1.5E+04 | 54 | 8.1E+05 |
| A | T | 3.3E+02 | n/a | n/a | 1 | 1.6E+04 | 49 | 7.9E+05 |
| M | M | 1.0E+03 | 2.7E+02 | 27% | 2 | 2.8E+04 | 27 | 7.6E+05 |
| Y | W | 5.4E+01 | 1.2E+01 | 23% | 2 | 6.4E+03 | 118 | 7.6E+05 |
| L | A | 2.3E+02 | 1.6E+01 | 7% | 2 | 1.3E+04 | 57 | 7.5E+05 |
| I | W | 1.5E+03 | 1.2E+00 | 0% | 2 | 3.3E+04 | 22 | 7.2E+05 |
| F | V | 7.1E+01 | 9.8E+00 | 14% | 2 | 7.1E+03 | 99 | 7.0E+05 |
| W | H | 4.2E+01 | 7.9E+00 | 19% | 2 | 5.4E+03 | 127 | 6.8E+05 |
| F | M | 8.4E+02 | 3.9E+01 | 5% | 2 | 2.4E+04 | 28 | 6.8E+05 |
| A | I | 2.2E+02 | 3.4E+01 | 16% | 2 | 1.2E+04 | 55 | 6.6E+05 |
| V | S | 8.8E+01 | 1.0E+01 | 11% | 2 | 7.6E+03 | 86 | 6.5E+05 |
| W | R | 8.0E+02 | 2.7E+01 | 3% | 2 | 2.3E+04 | 28 | 6.4E+05 |
| M | G | 5.4E+01 | 8.9E+00 | 16% | 2 | 5.9E+03 | 108 | 6.4E+05 |
| L | S | 6.9E+02 | 6.8E+01 | 10% | 2 | 2.1E+04 | 30 | 6.3E+05 |
| P | Q | 1.1E+03 | 2.3E+02 | 21% | 2 | 2.6E+04 | 23 | 6.2E+05 |
| P | V | 2.8E+02 | 4.8E+01 | 17% | 2 | 1.3E+04 | 47 | 6.1E+05 |
| F | A | 1.3E+02 | 1.6E+02 | 123% | 2 | 8.9E+03 | 68 | 6.1E+05 |
| V | R | 4.3E+02 | 1.8E+01 | 4% | 2 | 1.6E+04 | 37 | 6.0E+05 |
| M | S | 5.7E+02 | 4.2E+01 | 7% | 2 | 1.8E+04 | 33 | 6.0E+05 |
| P | H | 3.6E+02 | 6.9E+01 | 19% | 2 | 1.5E+04 | 41 | 5.9E+05 |
| Q | K | 4.9E+01 | 7.7E+00 | 16% | 2 | 5.3E+03 | 108 | 5.8E+05 |
| P | R | 5.0E+02 | 1.7E+01 | 3% | 2 | 1.7E+04 | 34 | 5.6E+05 |
| P | W | 1.3E+03 | 3.7E+01 | 3% | 2 | 2.7E+04 | 21 | 5.6E+05 |
| P | T | 8.4E+02 | 3.0E+01 | 4% | 2 | 2.2E+04 | 26 | 5.6E+05 |
| Y | H | 4.3E+01 | 5.7E+00 | 13% | 2 | 4.8E+03 | 114 | 5.5E+05 |
| P | I | 5.9E+02 | 4.5E+01 | 8% | 2 | 1.8E+04 | 30 | 5.4E+05 |
| V | G | 4.9E+01 | 1.3E+00 | 3% | 2 | 5.1E+03 | 103 | 5.2E+05 |
| P | G | 1.0E+02 | 9.9E+00 | 10% | 2 | 7.2E+03 | 71 | 5.1E+05 |
| F | S | 1.9E+02 | 2.5E+02 | 132% | 2 | 9.9E+03 | 51 | 5.1E+05 |
| M | W | 6.3E+02 | 3.4E+01 | 5% | 2 | 1.8E+04 | 28 | 5.0E+05 |
| A | W | 4.6E+02 | 3.8E+01 | 8% | 2 | 1.5E+04 | 32 | 4.7E+05 |
| L | G | 1.4E+02 | 3.5E+01 | 25% | 2 | 8.1E+03 | 58 | 4.7E+05 |
| Y | A | 4.9E+01 | 1.3E+00 | 3% | 2 | 4.8E+03 | 98 | 4.7E+05 |
| W | N | 2.2E+01 | 2.4E+00 | 11% | 2 | 3.2E+03 | 145 | 4.7E+05 |
| I | G | 1.7E+02 | 1.7E+01 | 10% | 2 | 8.6E+03 | 51 | 4.4E+05 |
| L | L | 5.9E+02 | 1.2E+01 | 2% | 2 | 1.6E+04 | 27 | 4.2E+05 |
| A | H | 1.4E+02 | 3.8E+01 | 27% | 2 | 7.4E+03 | 52 | 3.9E+05 |
| V | N | 7.0E+01 | 1.2E+01 | 17% | 2 | 5.2E+03 | 74 | 3.8E+05 |
| M | A | 3.6E+02 | 3.3E+01 | 9% | 2 | 1.2E+04 | 32 | 3.8E+05 |
| T | W | 2.1E+01 | 4.9E+00 | 24% | 2 | 2.7E+03 | 132 | 3.6E+05 |
| P | S | 4.2E+02 | 1.0E+01 | 2% | 2 | 1.2E+04 | 28 | 3.3E+05 |
| Y | G | 4.1E+01 | 5.6E+00 | 14% | 2 | 3.7E+03 | 88 | 3.2E+05 |
| A | Q | 4.6E+02 | 5.1E+01 | 11% | 2 | 1.2E+04 | 27 | 3.2E+05 |
| W | Y | 6.0E+01 | 8.6E+00 | 14% | 2 | 4.3E+03 | 73 | 3.2E+05 |
| F | I | 1.5E+02 | 2.0E+01 | 13% | 2 | 7.0E+03 | 45 | 3.2E+05 |
| I | S | 6.1E+02 | 2.6E+01 | 4% | 2 | 1.4E+04 | 23 | 3.1E+05 |
| I | L | 7.0E+02 | 1.0E+02 | 15% | 2 | 1.5E+04 | 21 | 3.1E+05 |
| Y | Y | 4.8E+01 | 3.1E+00 | 6% | 2 | 3.8E+03 | 79 | 3.0E+05 |
| P | A | 1.7E+02 | 6.7E+01 | 38% | 2 | 7.0E+03 | 40 | 2.8E+05 |
| V | A | 6.2E+01 | 2.8E+01 | 46% | 2 | 4.2E+03 | 67 | 2.8E+05 |
| L | E | 6.9E+02 | 1.6E+02 | 23% | 2 | 1.4E+04 | 20 | 2.8E+05 |
| V | E | 1.1E+02 | 8.0E+00 | 7% | 2 | 5.4E+03 | 50 | 2.7E+05 |
| A | S | 3.1E+02 | 7.1E+00 | 2% | 2 | 9.1E+03 | 29 | 2.7E+05 |
| W | M | 2.0E+02 | 2.3E+01 | 12% | 2 | 7.2E+03 | 37 | 2.6E+05 |
| M | P | 4.1E+00 | 1.2E+00 | 29% | 2 | 9.9E+02 | 241 | 2.4E+05 |
| F | E | 1.4E+02 | 4.6E+01 | 33% | 2 | 5.6E+03 | 40 | 2.2E+05 |
| M | L | 5.3E+02 | 2.4E+01 | 5% | 2 | 1.1E+04 | 20 | 2.2E+05 |
| I | P | 8.5E+00 | 8.1E−01 | 9% | 2 | 1.3E+03 | 157 | 2.1E+05 |
| Y | F | 3.4E+01 | 2.0E+00 | 6% | 2 | 2.6E+03 | 77 | 2.0E+05 |
| A | N | 7.8E+01 | 8.5E+00 | 11% | 2 | 4.0E+03 | 51 | 2.0E+05 |
| W | W | 7.0E+01 | 9.2E+00 | 13% | 2 | 3.8E+03 | 54 | 2.0E+05 |
| D | H | 5.0E+00 | 1.8E−02 | 0% | 2 | 1.0E+03 | 200 | 2.0E+05 |
| L | N | 9.4E+02 | 7.4E+01 | 8% | 2 | 1.4E+04 | 14 | 2.0E+05 |
| P | M | 9.9E+02 | 7.4E+01 | 7% | 2 | 1.4E+04 | 14 | 1.9E+05 |
| F | N | 2.0E+02 | 1.6E+02 | 81% | 2 | 6.2E+03 | 31 | 1.9E+05 |
| W | E | 2.4E+01 | 5.5E+00 | 23% | 2 | 2.1E+03 | 89 | 1.9E+05 |
| W | V | 8.6E+01 | 1.2E+00 | 1% | 2 | 3.9E+03 | 46 | 1.8E+05 |
| P | L | 3.3E+02 | 4.0E+01 | 12% | 2 | 7.7E+03 | 23 | 1.8E+05 |
| A | M | 4.6E+02 | 1.4E+02 | 31% | 2 | 9.0E+03 | 20 | 1.8E+05 |
| F | L | 5.3E+02 | 1.6E+02 | 30% | 2 | 9.5E+03 | 18 | 1.7E+05 |
| M | N | 4.3E+02 | 1.8E+01 | 4% | 2 | 8.3E+03 | 19 | 1.6E+05 |
| H | H | 2.5E+01 | 3.7E+00 | 15% | 2 | 2.0E+03 | 78 | 1.5E+05 |
| Y | R | 6.1E+02 | 1.8E+02 | 29% | 2 | 9.5E+03 | 16 | 1.5E+05 |
| T | R | 3.7E+01 | 5.2E+01 | 141% | 2 | 2.3E+03 | 63 | 1.4E+05 |
| Q | R | 6.6E+02 | 2.4E+01 | 4% | 2 | 9.6E+03 | 15 | 1.4E+05 |
| A | L | 3.0E+02 | 6.5E+01 | 22% | 2 | 6.3E+03 | 21 | 1.3E+05 |
| G | T | 5.9E+01 | 1.3E+01 | 22% | 2 | 2.7E+03 | 46 | 1.3E+05 |
| T | H | 1.4E+01 | 9.1E−01 | 7% | 2 | 1.3E+03 | 95 | 1.2E+05 |
| T | M | 1.3E+01 | 1.8E+01 | 141% | 2 | 1.2E+03 | 99 | 1.2E+05 |
| I | N | 8.7E+02 | 8.6E+01 | 10% | 2 | 1.0E+04 | 12 | 1.2E+05 |
| W | F | 4.9E+01 | 7.6E+00 | 15% | 2 | 2.4E+03 | 49 | 1.2E+05 |
| I | A | 6.5E+02 | 4.0E+02 | 62% | 2 | 8.7E+03 | 14 | 1.2E+05 |
| T | Q | 1.7E+01 | 6.4E+00 | 38% | 2 | 1.4E+03 | 82 | 1.2E+05 |
| Y | M | 1.6E+02 | 1.5E+02 | 98% | 2 | 4.3E+03 | 27 | 1.2E+05 |
| Q | Y | 2.0E+02 | 1.1E+01 | 5% | 2 | 4.7E+03 | 23 | 1.1E+05 |
| G | W | 8.2E+01 | 7.0E+00 | 8% | 2 | 2.9E+03 | 36 | 1.0E+05 |
| I | E | 1.3E+03 | 9.8E+01 | 7% | 2 | 1.2E+04 | 9 | 1.0E+05 |
| Q | I | 6.3E+02 | 9.1E+01 | 15% | 2 | 7.9E+03 | 13 | 9.8E+04 |
| Q | H | 5.5E+02 | 1.8E+01 | 3% | 2 | 7.3E+03 | 13 | 9.7E+04 |
| Y | N | 5.0E+01 | 6.8E+00 | 14% | 2 | 2.2E+03 | 44 | 9.6E+04 |
| T | T | 1.7E+01 | 9.4E+00 | 54% | 2 | 1.3E+03 | 74 | 9.4E+04 |
| T | D | 1.1E+01 | 8.9E−01 | 8% | 2 | 1.0E+03 | 91 | 9.3E+04 |
| S | K | 1.3E+01 | 1.1E+00 | 9% | 2 | 1.1E+03 | 85 | 9.3E+04 |
| T | K | 1.4E+01 | 1.9E−01 | 1% | 2 | 1.1E+03 | 81 | 9.1E+04 |
| Q | F | 1.5E+02 | 1.9E+01 | 13% | 2 | 3.6E+03 | 25 | 9.1E+04 |
| S | Y | 1.3E+01 | 4.4E+00 | 34% | 2 | 1.1E+03 | 82 | 8.7E+04 |
| F | P | 6.2E+00 | 2.4E+00 | 39% | 2 | 7.3E+02 | 118 | 8.6E+04 |
| T | Y | 2.3E+01 | 5.3E+00 | 23% | 2 | 1.4E+03 | 60 | 8.3E+04 |
| W | D | 1.6E+01 | 7.7E−01 | 5% | 2 | 1.2E+03 | 71 | 8.2E+04 |
| H | P | 7.0E+00 | 1.5E+00 | 21% | 2 | 7.4E+02 | 106 | 7.9E+04 |
| A | D | 5.6E+01 | 2.5E+01 | 45% | 2 | 2.1E+03 | 37 | 7.8E+04 |
| P | N | 5.8E+02 | 3.4E+01 | 6% | 2 | 6.7E+03 | 12 | 7.7E+04 |
| M | E | 6.0E+02 | 5.0E+01 | 8% | 2 | 6.8E+03 | 11 | 7.6E+04 |
| V | L | 3.3E+02 | 2.2E+01 | 7% | 2 | 5.0E+03 | 15 | 7.5E+04 |
| Q | Q | 1.4E+03 | 7.6E+01 | 5% | 2 | 1.0E+04 | 7 | 7.3E+04 |
| Q | V | 4.3E+02 | 1.6E+02 | 37% | 2 | 5.4E+03 | 13 | 6.8E+04 |
| Y | I | 6.6E+01 | 2.1E+01 | 31% | 2 | 2.1E+03 | 31 | 6.4E+04 |
| S | T | 3.2E+01 | 2.2E+00 | 7% | 2 | 1.4E+03 | 45 | 6.4E+04 |
| G | Y | 3.4E+01 | 1.2E+00 | 4% | 2 | 1.5E+03 | 43 | 6.3E+04 |
| Y | V | 4.9E+01 | 6.3E+00 | 13% | 2 | 1.7E+03 | 35 | 6.2E+04 |
| E | H | 7.7E+00 | 5.9E−01 | 8% | 2 | 6.9E+02 | 89 | 6.2E+04 |
| A | E | 1.8E+02 | 3.4E+00 | 2% | 2 | 3.3E+03 | 18 | 6.0E+04 |
| Q | T | 9.2E+02 | 1.5E+02 | 17% | 2 | 7.4E+03 | 8 | 5.9E+04 |
| K | D | 4.5E+01 | 2.8E+00 | 6% | 2 | 1.6E+03 | 36 | 5.9E+04 |
| G | R | 1.3E+02 | 4.3E+01 | 34% | 2 | 2.7E+03 | 21 | 5.6E+04 |
| S | W | 4.3E+01 | 2.9E+00 | 7% | 2 | 1.5E+03 | 36 | 5.5E+04 |
| P | E | 1.0E+03 | 1.4E+01 | 1% | 2 | 7.5E+03 | 7 | 5.4E+04 |
| E | F | 1.4E+01 | 1.2E+00 | 8% | 2 | 8.7E+02 | 63 | 5.4E+04 |
| E | K | 7.9E+00 | 3.5E−01 | 4% | 2 | 6.5E+02 | 83 | 5.4E+04 |
| S | D | 1.0E+01 | 1.4E+00 | 14% | 2 | 7.4E+02 | 72 | 5.3E+04 |
| P | P | 1.4E+01 | 3.2E+00 | 24% | 2 | 8.5E+02 | 63 | 5.3E+04 |
| S | F | 1.7E+01 | 4.2E+00 | 26% | 2 | 9.4E+02 | 57 | 5.3E+04 |
| N | T | 1.5E+01 | 4.0E+00 | 27% | 2 | 8.7E+02 | 60 | 5.2E+04 |
| Y | E | 5.4E+01 | 3.2E+00 | 6% | 2 | 1.7E+03 | 31 | 5.1E+04 |
| H | K | 2.8E+01 | 1.5E+01 | 55% | 2 | 1.2E+03 | 43 | 5.0E+04 |
| T | S | 1.6E+01 | 5.8E+00 | 37% | 2 | 8.9E+02 | 56 | 5.0E+04 |
| T | V | 2.0E+01 | 1.3E+01 | 66% | 2 | 9.8E+02 | 50 | 4.9E+04 |
| S | H | 2.4E+01 | 3.9E+00 | 17% | 2 | 1.1E+03 | 44 | 4.7E+04 |
| H | T | 5.0E+01 | 4.5E+00 | 9% | 2 | 1.5E+03 | 30 | 4.6E+04 |
| G | K | 3.4E+01 | 2.8E+00 | 8% | 2 | 1.2E+03 | 37 | 4.5E+04 |
| Q | W | 1.4E+03 | 6.3E+01 | 5% | 2 | 7.8E+03 | 6 | 4.4E+04 |
| N | F | 1.4E+01 | 1.8E+00 | 13% | 2 | 7.8E+02 | 56 | 4.3E+04 |
| G | G | 1.6E+01 | 2.9E−01 | 2% | 2 | 8.2E+02 | 51 | 4.2E+04 |
| N | K | 1.4E+01 | 4.1E−01 | 3% | 2 | 7.5E+02 | 55 | 4.1E+04 |
| N | Y | 1.5E+01 | 2.8E+00 | 18% | 2 | 7.8E+02 | 52 | 4.1E+04 |
| S | R | 1.3E+02 | 7.2E+01 | 55% | 2 | 2.3E+03 | 18 | 4.0E+04 |
| H | Y | 4.1E+01 | 9.4E+00 | 23% | 2 | 1.3E+03 | 31 | 3.9E+04 |
| G | F | 3.5E+01 | 1.3E+01 | 36% | 2 | 1.2E+03 | 33 | 3.8E+04 |
| E | A | 4.3E+00 | 1.9E−01 | 4% | 2 | 4.0E+02 | 92 | 3.7E+04 |
| D | F | 1.1E+01 | 1.5E+00 | 14% | 2 | 6.0E+02 | 57 | 3.4E+04 |
| G | S | 4.9E+01 | 1.0E+01 | 20% | 2 | 1.3E+03 | 26 | 3.4E+04 |
| G | V | 4.0E+01 | 6.2E−01 | 2% | 2 | 1.2E+03 | 29 | 3.3E+04 |
| S | A | 1.9E+01 | 7.9E−02 | 0% | 2 | 7.9E+02 | 41 | 3.3E+04 |
| T | E | 1.5E+01 | 4.7E−01 | 3% | 2 | 7.0E+02 | 47 | 3.3E+04 |
| H | R | 1.4E+02 | 8.9E+01 | 62% | 2 | 2.2E+03 | 15 | 3.2E+04 |
| S | Q | 4.5E+01 | 2.7E+00 | 6% | 2 | 1.2E+03 | 27 | 3.2E+04 |
| Q | M | 2.2E+03 | 1.2E+02 | 5% | 2 | 8.1E+03 | 4 | 3.1E+04 |
| T | G | 2.1E+01 | 1.0E+00 | 5% | 2 | 7.7E+02 | 37 | 2.9E+04 |
| N | H | 1.5E+01 | 4.2E+00 | 28% | 2 | 6.6E+02 | 43 | 2.8E+04 |
| T | N | 1.3E+01 | 1.0E−03 | 0% | 2 | 6.0E+02 | 46 | 2.8E+04 |
| N | G | 9.2E+00 | 4.5E−01 | 5% | 2 | 5.0E+02 | 55 | 2.8E+04 |
| G | M | 5.2E+01 | 2.3E+00 | 5% | 2 | 1.2E+03 | 23 | 2.8E+04 |
| S | S | 4.0E+01 | 2.0E+00 | 5% | 2 | 1.0E+03 | 26 | 2.7E+04 |
| H | S | 6.0E+01 | 8.6E+00 | 14% | 2 | 1.3E+03 | 21 | 2.7E+04 |
| F | D | 6.3E+01 | 8.0E−01 | 1% | 2 | 1.3E+03 | 21 | 2.7E+04 |
| Q | L | 4.8E+02 | 1.9E+01 | 4% | 2 | 3.5E+03 | 7 | 2.5E+04 |
| H | F | 4.2E+01 | 2.6E+00 | 6% | 2 | 1.0E+03 | 25 | 2.5E+04 |
| H | W | 3.2E+02 | 4.6E+01 | 14% | 2 | 2.8E+03 | 9 | 2.5E+04 |
| G | H | 5.8E+01 | 7.1E−01 | 1% | 2 | 1.2E+03 | 21 | 2.5E+04 |
| S | G | 2.4E+01 | 8.1E−01 | 3% | 2 | 7.7E+02 | 32 | 2.5E+04 |
| N | S | 1.6E+01 | 1.5E+00 | 9% | 2 | 6.4E+02 | 39 | 2.5E+04 |
| G | P | 7.5E+00 | 1.4E+00 | 19% | 2 | 4.3E+02 | 57 | 2.4E+04 |
| T | I | 6.3E+01 | 5.7E+01 | 90% | 2 | 1.2E+03 | 19 | 2.2E+04 |
| H | G | 4.7E+01 | 6.8E+00 | 15% | 2 | 1.0E+03 | 22 | 2.2E+04 |
| Y | D | 3.6E+01 | 2.4E+00 | 6% | 2 | 8.9E+02 | 24 | 2.2E+04 |
| N | Q | 2.7E+01 | 1.0E+00 | 4% | 2 | 7.4E+02 | 28 | 2.1E+04 |
| S | V | 3.8E+01 | 1.3E+00 | 3% | 2 | 8.9E+02 | 23 | 2.1E+04 |
| S | I | 5.5E+01 | 1.6E+01 | 30% | 2 | 1.1E+03 | 19 | 2.1E+04 |
| D | G | 5.8E+00 | 6.3E−01 | 11% | 2 | 3.5E+02 | 59 | 2.1E+04 |
| T | A | 2.0E+01 | 5.7E−01 | 3% | 2 | 6.3E+02 | 32 | 2.0E+04 |
| G | Q | 9.4E+01 | 2.5E+01 | 27% | 2 | 1.4E+03 | 15 | 2.0E+04 |
| S | M | 4.6E+01 | 3.4E+00 | 7% | 2 | 9.6E+02 | 21 | 2.0E+04 |
| H | Q | 1.6E+02 | 9.8E+00 | 6% | 2 | 1.8E+03 | 11 | 2.0E+04 |
| N | M | 2.7E+01 | 7.5E−01 | 3% | 2 | 7.2E+02 | 27 | 1.9E+04 |
| W | I | 1.8E+02 | 1.9E+01 | 10% | 2 | 1.9E+03 | 10 | 1.9E+04 |
| E | T | 7.9E+00 | 6.8E−01 | 9% | 2 | 3.9E+02 | 49 | 1.9E+04 |
| G | I | 8.8E+01 | 8.8E+00 | 10% | 2 | 1.3E+03 | 15 | 1.9E+04 |
| T | F | 2.2E+01 | 8.4E+00 | 38% | 2 | 6.4E+02 | 29 | 1.9E+04 |
| E | W | 8.0E+00 | 1.2E+00 | 15% | 2 | 3.9E+02 | 48 | 1.9E+04 |
| G | A | 5.0E+01 | 2.3E+00 | 5% | 2 | 9.5E+02 | 19 | 1.8E+04 |
| N | V | 1.7E+01 | 1.0E+00 | 6% | 2 | 5.5E+02 | 32 | 1.8E+04 |
| H | A | 5.3E+01 | 5.7E+00 | 11% | 2 | 9.6E+02 | 18 | 1.7E+04 |
| V | D | 7.0E+01 | 5.1E+00 | 7% | 2 | 1.1E+03 | 16 | 1.7E+04 |
| K | F | 3.5E+01 | 3.9E+00 | 11% | 2 | 7.5E+02 | 22 | 1.6E+04 |
| S | P | 1.3E+01 | 2.9E+00 | 23% | 2 | 4.5E+02 | 36 | 1.6E+04 |
| D | Y | 1.1E+01 | 6.3E−01 | 6% | 2 | 4.1E+02 | 39 | 1.6E+04 |
| K | I | 6.2E+01 | 3.5E+00 | 6% | 2 | 9.9E+02 | 16 | 1.6E+04 |
| N | W | 8.6E+01 | 2.7E+01 | 32% | 2 | 1.2E+03 | 13 | 1.6E+04 |
| K | H | 5.6E+01 | 2.1E+01 | 38% | 2 | 9.0E+02 | 16 | 1.4E+04 |
| G | L | 7.8E+01 | 1.3E+01 | 16% | 2 | 1.1E+03 | 14 | 1.4E+04 |
| D | V | 1.5E+01 | 7.7E−01 | 5% | 2 | 4.6E+02 | 30 | 1.3E+04 |
| K | Y | 3.8E+01 | 7.6E+00 | 20% | 2 | 7.0E+02 | 19 | 1.3E+04 |
| Y | L | 3.4E+02 | 3.0E+02 | 89% | 2 | 2.1E+03 | 6 | 1.3E+04 |
| E | Y | 1.5E+01 | 3.1E+00 | 21% | 2 | 4.3E+02 | 29 | 1.2E+04 |
| D | Q | 2.2E+00 | 3.1E+00 | 141% | 2 | 1.6E+02 | 75 | 1.2E+04 |
| S | N | 2.4E+01 | 2.5E+00 | 10% | 2 | 5.4E+02 | 23 | 1.2E+04 |
| K | K | 2.5E+01 | 5.1E+00 | 21% | 2 | 5.2E+02 | 21 | 1.1E+04 |
| N | I | 3.8E+01 | 1.5E+01 | 40% | 2 | 6.4E+02 | 17 | 1.1E+04 |
| N | A | 1.7E+01 | 2.3E+00 | 13% | 2 | 4.3E+02 | 25 | 1.1E+04 |
| Q | S | 9.5E+02 | 1.2E+02 | 13% | 2 | 3.1E+03 | 3 | 1.0E+04 |
| L | D | 1.0E+03 | 4.1E+02 | 40% | 2 | 3.1E+03 | 3 | 9.6E+03 |
| N | R | 2.2E+02 | 8.7E+00 | 4% | 2 | 1.4E+03 | 7 | 9.5E+03 |
| G | N | 5.2E+01 | 6.8E+00 | 13% | 2 | 7.0E+02 | 14 | 9.5E+03 |
| D | T | 6.4E+00 | 3.8E−01 | 6% | 2 | 2.5E+02 | 38 | 9.5E+03 |
| K | W | 1.3E+02 | 4.1E+01 | 30% | 2 | 1.1E+03 | 8 | 9.4E+03 |
| T | P | 7.2E+01 | 9.4E+01 | 130% | 2 | 8.2E+02 | 11 | 9.3E+03 |
| R | D | 1.4E+03 | 2.3E+02 | 16% | 2 | 3.6E+03 | 3 | 9.1E+03 |
| H | N | 7.1E+01 | 6.3E+00 | 9% | 2 | 8.0E+02 | 11 | 9.0E+03 |
| K | L | 8.9E+01 | 1.7E+01 | 20% | 2 | 8.8E+02 | 10 | 8.7E+03 |
| H | I | 7.8E+01 | 2.1E+01 | 26% | 2 | 8.2E+02 | 10 | 8.6E+03 |
| Q | A | 4.1E+02 | 1.9E+02 | 46% | 2 | 1.8E+03 | 5 | 8.3E+03 |
| Q | G | 1.4E+02 | 4.0E+01 | 28% | 2 | 1.1E+03 | 7 | 7.8E+03 |
| S | L | 8.2E+01 | 2.5E+00 | 3% | 2 | 7.9E+02 | 10 | 7.7E+03 |
| Y | P | 2.0E+01 | 9.3E−01 | 5% | 2 | 3.8E+02 | 19 | 7.4E+03 |
| H | V | 3.9E+01 | 9.9E−01 | 3% | 2 | 5.4E+02 | 14 | 7.4E+03 |
| W | L | 4.6E+02 | 4.5E+01 | 10% | 2 | 1.8E+03 | 4 | 7.1E+03 |
| K | V | 5.0E+01 | 1.9E+00 | 4% | 2 | 5.9E+02 | 12 | 7.0E+03 |
| S | E | 2.2E+01 | 2.1E+00 | 9% | 2 | 3.9E+02 | 17 | 6.8E+03 |
| K | R | 9.1E+01 | 1.0E+01 | 11% | 2 | 7.8E+02 | 9 | 6.7E+03 |
| Q | N | 6.1E+02 | 3.1E+01 | 5% | 2 | 2.0E+03 | 3 | 6.3E+03 |
| K | G | 3.3E+01 | 5.6E−02 | 0% | 2 | 4.6E+02 | 14 | 6.2E+03 |
| H | M | 2.7E+02 | 6.3E+00 | 2% | 2 | 1.3E+03 | 5 | 6.0E+03 |
| N | N | 1.5E+01 | 4.1E−01 | 3% | 2 | 2.9E+02 | 19 | 5.5E+03 |
| M | D | 5.0E+02 | 6.3E+01 | 12% | 2 | 1.6E+03 | 3 | 5.2E+03 |
| K | T | 7.2E+01 | 6.8E+00 | 9% | 2 | 6.1E+02 | 8 | 5.1E+03 |
| P | D | 7.9E+02 | 3.4E+01 | 4% | 2 | 2.0E+03 | 2 | 4.9E+03 |
| E | S | 5.3E+00 | 1.4E+00 | 26% | 2 | 1.6E+02 | 30 | 4.7E+03 |
| K | M | 1.4E+02 | 7.3E+01 | 52% | 2 | 8.0E+02 | 6 | 4.5E+03 |
| T | L | 1.4E+02 | 1.1E+02 | 79% | 2 | 7.5E+02 | 6 | 4.1E+03 |
| E | R | 3.0E+02 | 2.7E+00 | 1% | 2 | 1.1E+03 | 4 | 4.1E+03 |
| G | D | 7.3E+01 | 1.6E+01 | 21% | 2 | 5.4E+02 | 7 | 4.0E+03 |
| K | Q | 1.0E+02 | 1.1E+01 | 11% | 2 | 6.4E+02 | 6 | 3.9E+03 |
| E | I | 5.5E+01 | 3.2E+01 | 59% | 2 | 4.6E+02 | 8 | 3.9E+03 |
| N | L | 1.1E+02 | 2.2E+01 | 20% | 2 | 6.5E+02 | 6 | 3.9E+03 |
| Q | E | 9.2E+02 | 2.4E+02 | 26% | 2 | 1.8E+03 | 2 | 3.6E+03 |
| G | E | 8.7E+01 | 1.8E+01 | 21% | 2 | 5.1E+02 | 6 | 3.0E+03 |
| I | D | 1.3E+03 | 6.0E+01 | 5% | 2 | 2.0E+03 | 1 | 3.0E+03 |
| H | D | 8.6E+01 | 5.7E−01 | 1% | 2 | 4.9E+02 | 6 | 2.8E+03 |
| K | S | 8.4E+01 | 9.6E−01 | 1% | 2 | 4.7E+02 | 6 | 2.6E+03 |
| K | A | 5.2E+01 | 2.2E+01 | 42% | 2 | 3.6E+02 | 7 | 2.5E+03 |
| K | N | 6.4E+01 | 1.3E+01 | 21% | 2 | 3.7E+02 | 6 | 2.1E+03 |
| E | M | 1.3E+01 | 6.1E−01 | 5% | 2 | 1.5E+02 | 12 | 1.9E+03 |
| E | V | 2.4E+01 | 1.7E−01 | 1% | 2 | 2.0E+02 | 9 | 1.8E+03 |
| D | M | 1.1E+01 | 1.8E+00 | 16% | 2 | 1.4E+02 | 12 | 1.7E+03 |
| D | L | 2.7E+01 | 1.4E+01 | 51% | 2 | 2.1E+02 | 8 | 1.6E+03 |
| H | L | 1.4E+02 | 1.0E+02 | 73% | 2 | 4.7E+02 | 3 | 1.6E+03 |
| V | P | 6.3E+00 | 1.7E+00 | 27% | 2 | 9.5E+01 | 15 | 1.4E+03 |
| R | I | 7.0E+02 | 3.4E+01 | 5% | 2 | 1.0E+03 | 1 | 1.4E+03 |
| R | G | 6.8E+02 | 6.1E+01 | 9% | 2 | 8.8E+02 | 1 | 1.2E+03 |
| K | E | 6.2E+01 | 2.8E+01 | 45% | 2 | 2.7E+02 | 4 | 1.2E+03 |
| N | E | 2.0E+01 | 5.1E−01 | 3% | 2 | 1.5E+02 | 7 | 1.1E+03 |
| R | R | 9.0E+02 | 1.7E+02 | 19% | 2 | 9.8E+02 | 1 | 1.1E+03 |
| E | Q | 1.1E+01 | 1.6E+00 | 14% | 2 | 1.1E+02 | 10 | 1.0E+03 |
| R | F | 7.3E+02 | 9.7E+01 | 13% | 2 | 7.7E+02 | 1 | 8.0E+02 |
| R | W | 3.0E+03 | 4.3E+02 | 14% | 2 | 1.5E+03 | 0 | 7.0E+02 |
| R | V | 5.1E+02 | 2.5E+02 | 49% | 2 | 5.9E+02 | 1 | 6.7E+02 |
| D | R | 1.9E+02 | 6.2E+00 | 3% | 2 | 3.4E+02 | 2 | 6.2E+02 |
| R | L | 1.4E+03 | 2.4E+02 | 18% | 2 | 9.2E+02 | 1 | 6.1E+02 |
| H | E | 9.6E+01 | 6.3E+00 | 7% | 2 | 2.3E+02 | 2 | 5.5E+02 |
| R | K | 6.4E+02 | 8.3E+01 | 13% | 2 | 5.9E+02 | 1 | 5.5E+02 |
| E | G | 1.8E+01 | 6.6E+00 | 36% | 2 | 9.8E+01 | 5 | 5.3E+02 |
| R | Y | 9.6E+02 | 1.1E+02 | 12% | 2 | 6.9E+02 | 1 | 5.0E+02 |
| E | L | 1.0E+02 | 9.3E+00 | 9% | 2 | 2.1E+02 | 2 | 4.2E+02 |
| Q | D | 6.8E+02 | 1.2E+01 | 2% | 2 | 5.0E+02 | 1 | 3.6E+02 |
| R | H | 2.6E+03 | 5.6E+02 | 21% | 2 | 9.6E+02 | 0 | 3.5E+02 |
| R | E | 2.4E+03 | 5.2E+02 | 22% | 2 | 8.7E+02 | 0 | 3.1E+02 |
| R | T | 1.5E+03 | 6.9E+01 | 5% | 2 | 6.4E+02 | 0 | 2.8E+02 |
| R | N | 1.7E+03 | 1.8E+02 | 11% | 2 | 6.7E+02 | 0 | 2.6E+02 |
| R | A | 2.0E+03 | 2.7E+02 | 14% | 2 | 6.7E+02 | 0 | 2.2E+02 |
| R | S | 2.7E+03 | 1.4E+02 | 5% | 2 | 6.6E+02 | 0 | 1.6E+02 |
| R | M | 4.6E+03 | 1.6E+03 | 35% | 2 | 7.9E+02 | 0 | 1.4E+02 |
| R | Q | 4.5E+03 | 3.9E+02 | 9% | 2 | 6.4E+02 | 0 | 9.1E+01 |
| E | N | 3.3E+00 | 3.3E−01 | 10% | 2 | 1.7E+01 | 5 | 8.8E+01 |
| L | P | 1.6E+00 | 2.3E+00 | 141% | 2 | 1.1E+01 | 7 | 7.5E+01 |
| D | S | 4.4E+00 | 6.8E−02 | 2% | 2 | 1.5E+01 | 3 | 4.9E+01 |
| D | W | 7.2E+00 | 1.9E+00 | 27% | 2 | 1.4E+01 | 2 | 2.8E+01 |
| N | P | 2.9E+00 | 1.1E−01 | 4% | 2 | 9.0E+00 | 3 | 2.7E+01 |
| D | I | 4.1E+01 | 4.0E+00 | 10% | 2 | 3.0E+01 | 1 | 2.2E+01 |
| D | K | 1.1E+01 | 1.8E+00 | 17% | 2 | 1.0E+01 | 1 | 9.9E+00 |
| N | D | 3.0E+01 | 2.1E+01 | 70% | 2 | 3.0E+00 | 0 | 3.0E−01 |
| D | P | 1.6E+00 | 8.7E−01 | 55% | 2 | 6.6E−04 | 0 | 2.8E−07 |
| K | P | 8.7E+00 | 1.5E+00 | 18% | 2 | 5.6E−04 | 0 | 3.6E−08 |
| Q | P | 9.5E+00 | 2.6E+00 | 28% | 2 | 0.0E+00 | 0 | 0.0E+00 |

TABLE 5
Rationally Designed (Natural Sequence) Quenched Fluorescence Library

| | | | | | | | | | | | FXa | | | FIIa | Specificity | Functional Selectivity |
|---|---|---|---|---|---|---|---|---|---|---|---|---|---|---|---|---|
| Natural | | | | | | | | | | | k_{cat}/K_{M} | | | k_{cat}/K_{M} | Ratio | (Specificity*FIIa |
| Substrate | X₁₀ | X₉ | X₈ | X₇ | X₆ | X₅ | X₄ | X₃ | X₂ | X₁ | (M⁻¹s⁻¹) | % CV | n | (M⁻¹s⁻¹) | (FIIa/FXa) | k_{cat}/K_{M}) |
| FpA_P | D | F | L | A | E | G | G | G | P | R | 5.8E+00 | 31% | 3 | 4.0E+04 | 6914 | 2.8E+08 |
| Factor XI | N | E | S | T | T | K | I | K | P | R | 3.6E+01 | 20% | 3 | 2.0E+04 | 559 | 1.1E+07 |
| Factor V (2) | H | T | H | H | A | P | L | S | P | R | 1.0E+02 | 3% | 3 | 3.1E+04 | 302 | 9.5E+06 |
| Thrombin (3) | S | E | Y | Q | T | F | F | N | P | R | 1.4E+02 | 58% | 3 | 2.0E+04 | 141 | 2.8E+06 |
| Thrombin (1) | D | Q | V | T | V | A | M | T | P | R | 4.7E+02 | 13% | 3 | 3.0E+04 | 64 | 1.9E+06 |
| Factor XIII | T | V | E | L | Q | G | V | V | P | R | 1.0E+02 | 11% | 3 | 1.2E+04 | 114 | 1.3E+06 |
| PAR4 | S | T | P | S | I | L | P | A | P | R | 2.3E+02 | 11% | 3 | 9.4E+03 | 40 | 3.8E+05 |
| Protein C | E | D | Q | E | D | Q | V | D | P | R | 1.3E+01 | 5% | 3 | 1.8E+03 | 133 | 2.4E+05 |
| Factor VIII (2) | L | S | K | N | N | A | I | E | P | R | 1.0E+03 | 6% | 3 | 1.3E+04 | 13 | 1.7E+05 |
| Osteopontin | R | G | D | S | V | V | Y | G | L | R | 1.7E+00 | 87% | 3 | 4.1E+02 | 239 | 9.8E+04 |
| TAFI | Q | I | S | N | D | T | V | S | P | R | 3.4E+02 | 30% | 3 | 5.3E+03 | 16 | 8.3E+04 |
| FpA | D | F | L | A | E | G | G | G | V | R | 3.4E+00 | 2% | 3 | 4.1E+02 | 121 | 4.9E+04 |
| PAR1 (2) | — | — | T | N | A | T | L | D | P | R | 8.6E+02 | 1% | 3 | 3.6E+03 | 4 | 1.5E+04 |
| PAR1 (3) | — | — | — | — | A | T | L | D | P | R | 9.4E+02 | 8% | 3 | 3.3E+03 | 4 | 1.2E+04 |
| Factor VIII (3) | Y | D | E | D | E | N | Q | S | P | R | 3.2E+02 | 18% | 3 | 9.3E+02 | 3 | 2.7E+03 |
| Factor V (1) | N | R | L | A | A | A | L | G | I | R | 3.5E+02 | 79% | 3 | 7.2E+02 | 2 | 1.5E+03 |
| Factor V (3) | P | D | N | I | A | A | W | Y | L | R | 1.5E+01 | 57% | 3 | 9.8E+01 | 7 | 6.6E+02 |
| FpB | D | N | E | E | G | F | F | S | A | R | 2.3E+02 | 5% | 3 | 3.0E+02 | 1 | 3.9E+02 |
| PAR3 | N | L | A | K | P | T | L | P | I | K | 3.0E+00 | 5% | 3 | 1.6E+01 | 5 | 8.8E+01 |
| Antithrombin | A | S | T | A | V | V | I | A | G | R | 2.1E+00 | 51% | 3 | 3.1E−01 | 0 | 4.5E−02 |
| Thrombin (2) | E | D | S | D | R | A | I | E | G | R | 1.9E+03 | 10% | 3 | 0.0E+00 | 0 | 0.0E+00 |

### Example 5

### Screening the Thrombin Sensitive FX Molecules to Evaluate the Kinetic Rate Constant for Activation by Thrombin, FXa and FVIIa

In order to determine the activation rates for engineered thrombin sensitive FX molecules, a progress curve protocol will be used for evaluating the kinetics of activation by thrombin, FXa and FVIIa. The progress curve method assumes that the reaction follows a simple Michaelis Menten mechanism with the encounter complex of substrate (e.g. FX molecule) and enzyme (e.g. the activating protease) being limiting (i.e. psuedo-1^{st}-order). Under conditions where [FX molecules]<<K_{M} this method allows for an estimation of the k_{cat}/K_{M} from an exponential fit of the complete reaction progress curves (i.e. complete FX activation over time). The method will be carried out essentially as described by Louvain-Quintard et al. ((2005) JBC, 280: 41352-41359) with minor modifications. Briefly, thrombin sensitive FX molecules (˜10-50 nM) will be diluted in assay buffer (50 mM Hepes, 150 mM NaCl, 10 mM CaCl₂, 0.1% PEG8000, 0.1% BSA, pH 7.4) at 37° C. Activation reactions will be triggered by the addition of human α-thrombin to a final concentration of 2-5 nM. At timed intervals, samples will be removed and quenched with excess hirudin (100-200 nM) for thrombin reactions, EDTA for FVIIa reactions or ecotin, a specific FXa inhibitor, in slight excess for FXa reactions. The progress of FX activation to FXa will be followed by measuring the FXa activity of the quenched samples using a specific fluorogenic FXa substrate, Pefafluor Xa (Pentapharm, Switzerland), and by comparison to a standard curve of known amounts of FXa. Progress curves will be fit to the following equation:

Y=FXa₀+FXaₘₐₓ*(1−exp(−E*k*x))

where x=reaction time, FXa₀=the initial amount of FXa in the sample (if any), FXaₘₐₓ=the maximum amount of FXa at complete activation, k=the kinetic rate constant for activation in the form of k_{cat}/K_{M} with the units of M⁻¹s⁻¹ and E=the enzyme concentration in M units.

### Example 6

### Prediction of Binding to Major Histocompatibility Complex Class II (MHCII) Molecules

In silico prediction of binding of the sequences listed in Table 3 to MHCII molecules was performed using the NetMHCIIpan-2.0 software described in (Nielsen et al. (2010) Immunome research, 6(1), 9). A set of 376 amino acid sequences were constructed from a framework of DFNQTQPERGDNN(X₆)(X₅)AT(X₄)(X₃)(X₂)Rivgggeckdgecpwq (SEQ ID NO: 242) in which X₂ was proline, X₆ was alanine, X₅ was threonine and X₄ and X₃ were selected from A, D, E, F, G, H, I, K, L, M, N, P, Q, R, S, T, V, W and Y. All sequences were analysed using NetMHCIIPan-2.0 software using a Western European population for MHCII frequencies and a cut-off of 50. Only the results of the best predicted bindings (lowest ranking) are shown in in Table 6. The rank represents the top percentile of the query core peptide compared to 200,000 random natural peptides. For example, a rank of 3 indicates the query peptide as being among the top 3% of random peptides with respect to binding to the specific MHCII molecule. A cut-off below 3 was considered of a significant binding. For comparison, Factor X harbouring the FpA insert (SEQ ID NO: 3) had a predicted rank (2) below cut-off against MHC II molecule HLA-DQA10501-DQB10301.

None of the sequences listed in Table 3 had a rank below 3 indicating that the strategy of introducing optimising thrombin cleavage rate by an X₂ proline, X₆ alanine, X₅ being a threonine and X₄ and X₃ being selected from A, D, E, F, G, H, I, K, L, M, N, P, Q, R, S, T, V, W and Y was not predicted to produce novel MHCII binding peptides. Thus, by using this in silico approach, introducing a thrombin sensitive cleavage sequence into Factor X would not be expected to create an immunogenic molecule when using one of the best ranked sequences listed in Table 6.

TABLE 6
X₄/X₃ Positional Scanning Library for Predicted MHC II Binding
X₄/X₃ Positional Scanning Library for predicted MHC II binding: form
DFNQTQPERGDNN(X₆)(X₅)AT(X₄)(X₃)(X₂)Rivggqeckdgecpwq (SEQ
ID NO: 242) in which X₂ was proline, X₆ was Alanine, X₅ was threonine
and X₄ and X₃ were selected from A, D, E, F, G, H, I, K, L, M, N, P, Q,
R, S, T, V, W and Y. All amino acid variants herein can form part of
Factor X molecules according to the invention. For comparison, Factor
X harbouring the FpA insert (SEQ ID NO 3) had a predicted rank (2)
below cut-off against MHC II molecule HLA-DQA10501-DQB10301.

| X₄ | X₃ | MHC II molecule | Rank |
|---|---|---|---|
| L | Q | HLA-DQA10301-DQB10302 | 32 |
| L | M | HLA-DQA10102-DQB10602 | 8 |
| L | R | DRB1_1305 | 32 |
| I | Q | HLA-DQA10102-DQB10602 | 8 |
| L | T | DRB1_0901 | 32 |
| F | R | DRB1_0801 | 32 |
| I | M | HLA-DQA10102-DQB10602 | 8 |
| G | G | HLA-DQA10501-DQB10301 | 16 |
| I | R | DRB1_0804 | 32 |
| L | W | HLA-DQA10301-DQB10302 | 32 |
| M | R | DRB1_0806 | 32 |
| L | I | HLA-DQA10102-DQB10602 | 16 |
| I | W | HLA-DQA10301-DQB10302 | 32 |
| M | Q | HLA-DQA10102-DQB10602 | 32 |
| M | T | DRB1_1302 | 32 |
| I | T | DRB1_0804 | 32 |
| L | K | DRB1_0806 | 32 |
| L | S | HLA-DQA10301-DQB10302 | 32 |
| F | Q | HLA-DQA10301-DQB10302 | 32 |
| M | T | DRB1_1302 | 32 |
| L | V | HLA-DQA10102-DQB10602 | 16 |
| M | M | HLA-DQA10102-DQB10602 | 8 |
| F | T | HLA-DQA10401-DQB10402 | 32 |
| P | W | HLA-DQA10301-DQB10302 | 32 |
| P | Q | DRB1_0407 | 50 |
| I | I | DRB1_0407 | 50 |
| I | H | DRB1_0407 | 50 |
| L | H | DRB1_0701 | 50 |
| F | K | DRB1_0402 | 50 |
| F | M | HLA-DQA10102-DQB10602 | 16 |
| I | V | DRB1_0804 | 32 |
| I | K | DRB1_0407 | 50 |
| V | W | HLA-DQA10301-DQB10302 | 32 |
| W | R | HLA-DQA10301-DQB10302 | 32 |
| M | K | DRB1_0407 | 50 |
| P | T | DRB1_1603 | 50 |
| L | S | HLA-DQA10301-DQB10302 | 32 |
| V | Q | DRB1_1603 | 50 |
| L | Y | HLA-DQA10401-DQB10402 | 32 |
| I | K | DRB1_0407 | 50 |
| F | N | HLA-DQA10401-DQB104023 | 2 |
| F | W | HLA-DQA10401-DQB10402 | 32 |
| W | Q | HLA-DQA10301-DQB10302 | 32 |
| M | S | DRB1_1302 | 32 |
| I | Y | HLA-DQA10401-DQB10402 | 32 |
| P | I | DRB1_1603 | 50 |
| M | W | HLA-DQA10301-DQB10302 | 32 |
| M | H | DRB1_0407 | 50 |
| M | I | HLA-DQA10102-DQB10602 | 16 |
| F | H | DRB1_0802 | 50 |
| P | R | DRB1_0701 | 50 |
| M | V | HLA-DQA10102-DQB10602 | 16 |
| W | S | HLA-DQA10301-DQB10302 | 32 |
| W | T | HLA-DQA10401-DQB10402 | 32 |
| V | R | DRB1_0801 | 32 |
| V | T | DRB1_0801 | 32 |
| L | L | HLA-DQA10102-DQB10602 | 16 |
| L | F | HLA-DQA10401-DQB10402 | 32 |
| V | M | HLA-DQA10102-DQB10602 | 8 |
| I | L | DRB1_0407 | 50 |
| P | H | DRB1_1501 | 50 |
| L | E | HLA-DQA10401-DQB10402 | 32 |
| P | Y | DRB1_1603 | 50 |
| F | G | HLA-DQA10301-DQB10302 | 32 |
| I | S | DRB1_0804 | 32 |
| P | M | HLA-DQA10102-DQB10602 | 32 |
| L | N | DRB1_0402 | 32 |
| W | A | HLA-DQA10401-DQB10402 | 32 |
| I | F | HLA-DQA10501-DQB10301 | 32 |
| I | E | HLA-DQA10501-DQB10301 | 32 |
| P | V | DRB1_1501 | 50 |
| W | K | DRB1_0402 | 50 |
| V | I | HLA-DQA10102-DQB10602 | 16 |
| L | A | HLA-DQA10401-DQB10402 | 32 |
| V | H | DRB1_0406 | 50 |
| M | Y | HLA-DQA10301-DQB10302 | 32 |
| P | S | DRB1_1501 | 50 |
| V | V | DRB1_0801 | 32 |
| F | Y | HLA-DQA10401-DQB10402 | 32 |
| M | A | HLA-DQA10301-DQB10302 | 32 |
| I | E | HLA-DQA10501-DQB10301 | 32 |
| W | G | HLA-DQA10301-DQB10302 | 32 |
| V | Y | HLA-DQA10301-DQB10302 | 32 |
| V | V | DRB1_0801 | 32 |
| M | F | HLA-DQA10401-DQB10402 | 32 |
| M | L | HLA-DQA10102-DQB10602 | 16 |
| I | N | HLA-DQA10102-DQB10602 | 8 |
| Q | Q | DRB1_1201 | 50 |
| F | S | HLA-DQA10401-DQB10402 | 32 |
| V | K | HLA-DQA10102-DQB10602 | 16 |
| W | P | HLA-DQA10301-DQB10302 | 32 |
| Q | R | DRB1_1201 | 50 |
| F | F | HLA-DQA10101-DQB10501 | 32 |
| Y | R | DRB1_0402 | 50 |
| F | L | HLA-DQA10101-DQB10501 | 32 |
| P | A | HLA-DQA10301-DQB10302 | 32 |
| P | K | DRB1_1502 | 50 |
| P | F | HLA-DQA10401-DQB10402 | 32 |
| F | A | HLA-DQA10102-DQB10602 | 16 |
| I | A | HLA-DQA10501-DQB10301 | 32 |
| Y | Q | HLA-DQA10301-DQB10302 | 32 |
| I | G | HLA-DQA10501-DQB10301 | 32 |
| V | F | HLA-DQA10401-DQB10402 | 32 |
| M | N | DRB1_1302 | 32 |
| Q | M | HLA-DQA10401-DQB10402 | 32 |
| L | G | HLA-DQA10301-DQB10302 | 32 |
| Q | I | HLA-DQA10401-DQB10402 | 32 |
| Q | W | HLA-DQA10301-DQB10302 | 32 |
| P | L | DRB1_1603 | 50 |
| V | S | DRB1_0801 | 32 |
| P | E | HLA-DQA10301-DQB10302 | 32 |
| Y | T | HLA-DQA10301-DQB10302 | 32 |
| Q | T | HLA-DQA10301-DQB10302 | 32 |
| Q | H | DRB1_0402 | 50 |
| P | G | HLA-DQA10501-DQB10301 | 32 |
| W | M | HLA-DQA10401-DQB10402 | 32 |
| F | V | HLA-DQA10101-DQB10501 | 32 |
| P | A | HLA-DQA10301-DQB10302 | 32 |
| F | I | DRB1_0802 | 32 |
| M | E | HLA-DQA10401-DQB10402 | 32 |
| P | N | DRB1_1501 | 50 |
| Y | S | HLA-DQA10301-DQB10302 | 32 |
| Y | W | HLA-DQA10301-DQB10302 | 32 |
| F | N | HLA-DQA10401-DQB10402 | 32 |
| M | G | DRB1_0103 | 32 |
| F | E | HLA-DQA10401-DQB10402 | 32 |
| Q | V | HLA-DQA10401-DQB10402 | 32 |
| V | E | HLA-DQA10301-DQB10302 | 16 |
| W | H | HLA-DQA10301-DQB10302 | 32 |
| V | S | DRB1_0801 | 32 |
| Q | K | DRB1_1305 | 50 |
| V | N | HLA-DQA10102-DQB10602 | 8 |
| V | G | HLA-DQA10401-DQB10402 | 32 |
| V | L | HLA-DQA10102-DQB10602 | 16 |
| Y | K | DRB1_0101 | 50 |
| Y | H | DRB1_1501 | 50 |
| Y | A | HLA-DQA10401-DQB10402 | 32 |
| Q | Y | HLA-DQA10301-DQB10302 | 32 |
| W | Y | HLA-DQA10101-DQB10501 | 32 |
| Y | M | HLA-DQA10401-DQB10402 | 32 |
| V | A | DRB1_1302 | 32 |
| W | V | HLA-DQA10101-DQB10501 | 32 |
| L | D | DRB1_0301 | 8 |
| Y | Y | HLA-DQA10401-DQB10402 | 32 |
| W | W | HLA-DQA10101-DQB10501 | 32 |
| Y | G | HLA-DQA10301-DQB10302 | 32 |
| L | D | DRB1_0301 | 8 |
| Q | F | HLA-DQA10401-DQB10402 | 32 |
| R | D | HLA-DQA10401-DQB10402 | 32 |
| Q | L | HLA-DQA10401-DQB10402 | 32 |
| L | D | DRB1_0301 | 8 |
| W | N | HLA-DQA10301-DQB10302 | 32 |
| L | D | DRB1_0301 | 8 |
| Q | S | HLA-DQA10301-DQB10302 | 32 |
| L | D | DRB1_0301 | 8 |
| L | D | DRB1_0301 | 8 |
| L | D | DRB1_0301 | 8 |
| L | D | DRB1_0301 | 8 |
| L | D | DRB1_0301 | 8 |
| G | W | HLA-DQA10301-DQB10302 | 32 |
| L | D | DRB1_0301 | 8 |
| H | W | HLA-DQA10501-DQB10301 | 32 |
| R | P | DRB1_0407 | 50 |
| G | T | HLA-DQA10501-DQB10301 | 32 |
| L | D | DRB1_0301 | 8 |
| T | W | HLA-DQA10301-DQB10302 | 32 |
| L | D | DRB1_0301 | 8 |
| L | D | DRB1_0301 | 8 |
| G | R | HLA-DQA10501-DQB10301 | 32 |
| L | D | DRB1_0301 | 8 |
| Y | F | HLA-DQA10101-DQB10501 | 32 |
| L | D | DRB1_0301 | 8 |
| L | D | DRB1_0301 | 8 |
| W | F | HLA-DQA10101-DQB10501 | 32 |
| T | R | HLA-DQA10301-DQB10302 | 32 |
| S | R | HLA-DQA10501-DQB10301 | 32 |
| Y | N | DRB1_1302 | 32 |
| H | R | HLA-DQA10501-DQB10301 | 32 |
| W | E | HLA-DQA10301-DQB10302 | 16 |
| Y | L | DRB1_0802 | 32 |
| Y | I | DRB1_0802 | 32 |
| I | D | HLA-DQA10501-DQB10301 | 32 |
| H | H | HLA-DQA10501-DQB10301 | 32 |
| P | D | HLA-DQA10401-DQB10402 | 32 |
| Q | N | DRB1_1302 | 32 |
| W | I | HLA-DQA10401-DQB10402 | 32 |
| Q | A | HLA-DQA10301-DQB10302 | 32 |
| Q | E | HLA-DQA10401-DQB10402 | 32 |
| W | L | HLA-DQA10101-DQB10501 | 32 |
| V | D | DRB1_0301 | 16 |
| H | Q | HLA-DQA10501-DQB10301 | 32 |
| Y | V | DRB1_0802 | 32 |
| Y | E | HLA-DQA10401-DQB10402 | 32 |
| K | D | HLA-DQA10401-DQB10402 | 32 |
| M | D | DRB1_0301 | 10 |
| S | W | HLA-DQA10301-DQB10302 | 32 |
| H | T | HLA-DQA10501-DQB10301 | 32 |
| G | Y | HLA-DQA10501-DQB10301 | 32 |
| R | W | HLA-DQA10301-DQB10302 | 32 |
| N | R | HLA-DQA10301-DQB10302 | 32 |
| S | T | DRB1_1302 | 32 |
| T | Q | HLA-DQA10301-DQB10302 | 32 |
| T | Y | HLA-DQA10301-DQB10302 | 32 |
| G | Q | HLA-DQA10501-DQB10301 | 32 |
| I | P | HLA-DQA10102-DQB10602 | 8 |
| T | H | HLA-DQA10301-DQB10302 | 32 |
| G | S | HLA-DQA10501-DQB10301 | 16 |
| F | D | HLA-DQA10101-DQB10501 | 32 |
| G | I | HLA-DQA10501-DQB10301 | 32 |
| H | M | HLA-DQA10501-DQB10301 | 32 |
| T | T | HLA-DQA10301-DQB10302 | 32 |
| H | Y | HLA-DQA10501-DQB10301 | 32 |
| H | S | HLA-DQA10501-DQB10301 | 32 |
| T | M | HLA-DQA10301-DQB10302 | 32 |
| G | K | HLA-DQA10501-DQB10301 | 32 |
| G | H | HLA-DQA10501-DQB10301 | 32 |
| G | M | HLA-DQA10102-DQB10602 | 32 |
| S | Q | DRB1_0701 | 50 |
| H | K | HLA-DQA10501-DQB10301 | 32 |
| E | D | HLA-DQA10301-DQB10302 | 16 |
| T | I | HLA-DQA10301-DQB10302 | 32 |
| G | F | HLA-DQA10301-DQB10302 | 32 |
| W | D | HLA-DQA10301-DQB10302 | 16 |
| N | W | HLA-DQA10301-DQB10302 | 32 |
| G | V | HLA-DQA10501-DQB10301 | 32 |
| T | K | HLA-DQA10301-DQB10302 | 32 |
| K | W | DRB1_0302 | 50 |
| E | R | HLA-DQA10301-DQB10302 | 32 |
| V | D | DRB1_0301 | 16 |
| S | K | HLA-DQA10501-DQB10301 | 32 |
| S | Y | HLA-DQA10301-DQB10302 | 32 |
| S | I | DRB1_1302 | 32 |
| Q | G | HLA-DQA10301-DQB10302 | 32 |
| G | L | HLA-DQA10501-DQB10301 | 32 |
| S | H | DRB1_0701 | 50 |
| S | S | DRB1_1302 | 32 |
| H | F | HLA-DQA10501-DQB10301 | 32 |
| T | D | HLA-DQA10301-DQB10302 | 32 |
| H | G | HLA-DQA10501-DQB10301 | 32 |
| D | H | DRB1_1603 | 50 |
| R | I | DRB1_0804 | 32 |
| M | P | DRB1_0407 | 50 |
| K | I | DRB1_0806 | 32 |
| T | V | HLA-DQA10301-DQB10302 | 32 |
| R | R | DRB1_0806 | 32 |
| H | A | HLA-DQA10501-DQB10301 | 32 |
| S | M | HLA-DQA10401-DQB10402 | 32 |
| R | H | DRB1_0406 | 50 |
| G | A | HLA-DQA10501-DQB10301 | 16 |
| S | F | HLA-DQA10401-DQB10402 | 32 |
| Q | S | HLA-DQA10301-DQB10302 | 32 |
| R | L | HLA-DQA10102-DQB10602 | 32 |
| K | H | DRB1_0407 | 50 |
| T | S | HLA-DQA10301-DQB10302 | 32 |
| S | V | DRB1_1302 | 32 |
| Y | D | HLA-DQA10101-DQB10501 | 32 |
| R | G | HLA-DQA10102-DQB10602 | 32 |
| K | L | DRB1_0801 | 32 |
| N | T | HLA-DQA10301-DQB10302 | 32 |
| R | E | HLA-DQA10301-DQB10302 | 32 |
| E | F | HLA-DQA10301-DQB10302 | 32 |
| P | P | DRB1_1603 | 50 |
| G | G | HLA-DQA10501-DQB10301 | 16 |
| T | P | HLA-DQA10301-DQB10302 | 32 |
| H | I | HLA-DQA10501-DQB10301 | 32 |
| H | N | HLA-DQA10501-DQB10301 | 32 |
| K | M | HLA-DQA10102-DQB10602 | 32 |
| S | L | HLA-DQA10102-DQB10602 | 32 |
| R | M | HLA-DQA10102-DQB10602 | 32 |
| S | A | HLA-DQA10102-DQB10602 | 16 |
| N | Y | HLA-DQA10301-DQB10302 | 32 |
| N | F | HLA-DQA10301-DQB10302 | 32 |
| K | R | DRB1_0407 | 50 |
| S | G | HLA-DQA10301-DQB10302 | 32 |
| T | G | HLA-DQA10301-DQB10302 | 32 |
| R | F | HLA-DQA10301-DQB10302 | 32 |
| K | F | HLA-DQA10101-DQB10501 | 50 |
| N | K | HLA-DQA10301-DQB10302 | 32 |
| T | L | HLA-DQA10301-DQB10302 | 32 |
| N | Q | HLA-DQA10301-DQB10302 | 32 |
| H | P | HLA-DQA10501-DQB10301 | 32 |
| S | D | DRB1_0301 | 32 |
| F | P | DRB1_0407 | 50 |
| N | M | HLA-DQA10301-DQB10302 | 32 |
| L | G | HLA-DQA10301-DQB10302 | 32 |
| K | Y | HLA-DQA10101-DQB10501 | 50 |
| G | N | HLA-DQA10501-DQB10301 | 32 |
| T | E | HLA-DQA10301-DQB10302 | 32 |
| R | Y | DRB1_0302 | 50 |
| E | H | HLA-DQA10401-DQB10402 | 32 |
| R | A | HLA-DQA10102-DQB10602 | 16 |
| R | N | DRB1_1302 | 32 |
| R | S | HLA-DQA10301-DQB10302 | 32 |
| N | H | HLA-DQA10301-DQB10302 | 32 |
| E | K | DRB1_1401 | 50 |
| N | L | HLA-DQA10301-DQB10302 | 32 |
| N | I | HLA-DQA10301-DQB10302 | 32 |
| R | Q | DRB1_0407 | 50 |
| T | F | HLA-DQA10301-DQB10302 | 32 |
| R | T | HLA-DQA10101-DQB10501 | 50 |
| N | S | HLA-DQA10301-DQB10302 | 32 |
| K | Q | DRB1_0406 | 50 |
| T | A | HLA-DQA10301-DQB10302 | 32 |
| K | T | DRB1_0406 | 50 |
| D | F | HLA-DQA10401-DQB10402 | 32 |
| T | N | HLA-DQA10301-DQB10302 | 32 |
| R | K | HLA-DQA10101-DQB10501 | 50 |
| K | V | HLA-DQA10101-DQB10501 | 50 |
| R | V | DRB1_1302 | 32 |
| N | V | HLA-DQA10301-DQB10302 | 32 |
| S | N | DRB1_0402 | 32 |
| G | D | HLA-DQA10401-DQB10402 | 32 |
| H | V | HLA-DQA10501-DQB10301 | 32 |
| K | K | HLA-DQA10501-DQB10201 | 50 |
| G | E | HLA-DQA10301-DQB10302 | 32 |
| N | G | HLA-DQA10301-DQB10302 | 32 |
| Q | D | HLA-DQA10401-DQB10402 | 32 |
| H | D | HLA-DQA10501-DQB10301 | 32 |
| H | L | HLA-DQA10501-DQB10301 | 32 |
| K | S | DRB1_0701 | 32 |
| E | I | HLA-DQA10401-DQB10402 | 32 |
| D | V | HLA-DQA10401-DQB10402 | 32 |
| K | G | HLA-DQA10102-DQB10602 | 32 |
| S | P | HLA-DQA10501-DQB10301 | 32 |
| N | A | HLA-DQA10301-DQB10302 | 32 |
| G | P | HLA-DQA10501-DQB10301 | 32 |
| E | Y | HLA-DQA10401-DQB10402 | 32 |
| D | Y | HLA-DQA10401-DQB10402 | 32 |
| Y | G | HLA-DQA10301-DQB10302 | 32 |
| G | G | HLA-DQA10501-DQB10301 | 16 |
| E | A | HLA-DQA10401-DQB10402 | 32 |
| S | E | HLA-DQA10401-DQB10402 | 32 |
| E | W | HLA-DQA10401-DQB10402 | 32 |
| E | T | HLA-DQA10401-DQB10402 | 32 |
| Y | P | DRB1_0406 | 50 |
| K | N | DRB1_1302 | 32 |
| K | A | HLA-DQA10301-DQB10302 | 32 |
| D | G | HLA-DQA10301-DQB10302 | 32 |
| D | R | DRB1_1502 | 50 |
| F | S | HLA-DQA10401-DQB10402 | 32 |
| N | N | HLA-DQA10301-DQB10302 | 32 |
| K | E | HLA-DQA10301-DQB10302 | 32 |
| D | T | HLA-DQA10401-DQB10402 | 32 |
| H | E | HLA-DQA10501-DQB10301 | 32 |
| D | L | HLA-DQA10301-DQB10302 | 32 |
| E | L | HLA-DQA10301-DQB10302 | 32 |
| E | V | HLA-DQA10401-DQB10402 | 32 |
| D | Q | HLA-DQA10301-DQB10302 | 32 |
| E | S | HLA-DQA10401-DQB10402 | 32 |
| E | M | HLA-DQA10301-DQB10302 | 32 |
| N | E | HLA-DQA10301-DQB10302 | 32 |
| D | M | HLA-DQA10401-DQB10402 | 32 |
| D | N | HLA-DQA10301-DQB10302 | 32 |
| E | Q | HLA-DQA10301-DQB10302 | 32 |
| W | Y | HLA-DQA10101-DQB10501 | 32 |
| E | G | HLA-DQA10301-DQB10302 | 32 |
| V | P | DRB1_1603 | 50 |
| D | I | HLA-DQA10301-DQB10302 | 32 |
| E | N | HLA-DQA10401-DQB10402 | 32 |
| L | P | DRB1_0407 | 50 |
| D | S | HLA-DQA10301-DQB10302 | 32 |
| E | E | HLA-DQA10301-DQB10302 | 16 |
| D | W | HLA-DQA10401-DQB10402 | 32 |
| L | P | DRB1_0407 | 50 |
| D | K | DRB1_1501 | 50 |
| N | P | HLA-DQA10301-DQB10302 | 32 |
| D | A | HLA-DQA10401-DQB10402 | 32 |
| N | D | HLA-DQA10301-DQB10302 | 32 |
| E | P | DRB1_1501 | 50 |
| I | A | HLA-DQA10501-DQB10301 | 32 |
| D | D | HLA-DQA10401-DQB10402 | 32 |
| D | P | DRB1_0406 | 50 |
| K | P | DRB1_0407 | 50 |
| D | E | HLA-DQA10401-DQB10402 | 32 |
| Q | P | DRB1_0407 | 50 |
| I | E | HLA-DQA10501-DQB10301 | 32 |

### Example 7

### Prediction of Factor X Peptide Binding to Major Histocompatibility Complex Class II (MHCII) Molecules

### A. Materials and Methods

In silico prediction of binding of thrombin sensitive Factor X molecules listed in table 8 to MHCII molecules (expressed from HLA-II alleles) was performed using the algorithm NetMHCIIpan 2.1 for HLA-DR predictions (Nielsen et al., (2010) Immunome Research, 6:9) and NetMHCII 2.2 for HLA-DP/DQ predictions (Nielsen et al., (2009) BMC Bioinformatics 10:296). The Immunogenicity Risk Score (IRS) was calculated as the sum of weighted peptide ranks multiplied by population frequency of MHCII/HLA-II alleles (listed in Table 7).

Ranks were assigned as follows: peptide/MHCII combinations with a rank equal to or below 1 was assigned a weight of 2, combinations with a rank above 1 but equal to or below 3 were assigned a weight of 0.5, and combinations with a rank above 3 but equal to or below 10 were assigned a weight of 0.2. Only novel peptides (not present in wild-type Factor X) with predicted ranks equal to or below 10 were included. Sums are reported separately for HLA-DR, HLA-DP and HLA-DQ loci.

TABLE 7
List of HLA-II Alleles and their Population
Frequency (in Westen European populations)
Used in the Prediction of Factor X Peptide
Binding to MHC-II Molecules

| HLA-II Allele | Population Frequency |
|---|---|
| DRB1_0101 | 0.0830 |
| DRB1_0102 | 0.0064 |
| DRB1_0103 | 0.0269 |
| DRB1_0301 | 0.1484 |
| DRB1_0302 | 0.0004 |
| DRB1_0401 | 0.1035 |
| DRB1_0402 | 0.0044 |
| DRB1_0403 | 0.0032 |
| DRB1_0404 | 0.0441 |
| DRB1_0405 | 0.0032 |
| DRB1_0406 | 0.0004 |
| DRB1_0407 | 0.0148 |
| DRB1_0408 | 0.0036 |
| DRB1_0416 | 0.0008 |
| DRB1_0701 | 0.1516 |
| DRB1_0801 | 0.0201 |
| DRB1_0802 | 0.0012 |
| DRB1_0803 | 0.0024 |
| DRB1_0804 | 0.0008 |
| DRB1_0806 | 0.0008 |
| DRB1_0901 | 0.0060 |
| DRB1_1001 | 0.0068 |
| DRB1_1101 | 0.0305 |
| DRB1_1102 | 0.0016 |
| DRB1_1103 | 0.0036 |
| DRB1_1104 | 0.0197 |
| DRB1_1201 | 0.0136 |
| DRB1_1202 | 0.0004 |
| DRB1_1301 | 0.0469 |
| DRB1_1302 | 0.0325 |
| DRB1_1303 | 0.0136 |
| DRB1_1305 | 0.0004 |
| DRB1_1327 | 0.0004 |
| DRB1_1401 | 0.0181 |
| DRB1_1501 | 0.1677 |
| DRB1_1502 | 0.0048 |
| DRB1_1601 | 0.0124 |
| DRB1_1602 | 0.0004 |
| DRB1_1603 | 0.0004 |
| HLA-DPA10103-DPB10201 | 0.2670 |
| HLA-DPA10103-DPB10401 | 0.6700 |
| HLA-DPA10201-DPB10101 | 0.0200 |
| HLA-DPA10201-DPB10501 | 0.0074 |
| HLA-DQA10101-DQB10501 | 0.0878 |
| HLA-DQA10102-DQB10602 | 0.0854 |
| HLA-DQA10301-DQB10302 | 0.0658 |
| HLA-DQA10401-DQB10402 | 0.0244 |
| HLA-DQA10501-DQB10201 | 0.1006 |
| HLA-DQA10501-DQB10301 | 0.1603 |

### B. Results

Table 8 below sets forth the predicted immunogenicity risk score of thrombin sensitive Factor X molecules. The total IRS score ranged from 0 to 0.98, indicating predicted differences in potential immunogenicity of the thrombin-sensitive Factor X molecules. When viewed in the context of the four protein design strategies outlined in Example 1, thrombin sensitive Factor X molecules designed by strategy 4 and most of the cleavage sequences designed by strategy 3 showed IRS scores of 0, suggesting a very low immunogenicity risk. Other thrombin sensitive Factor X molecules designed by strategy 3 demonstrated some elevated IRS scores in the 0.02-0.05 range, whereas Factor X molecules created by strategies 1 and 2 showed the greatest propensity for elevated IRS scores (up to 0.98). Thus, the Factor X molecules generated by the minimalistic approaches (strategies 3 and 4) are predicted to be less immunogenic when compared to Factor X molecules with larger amino acid insertions (strategies 1 and 2).

TABLE 8
In silico Predicted Immunogenicity Risk Score of Thrombin Sensitive FX Molecules Based on
Binding to MHC-II Molecules from HLA-DR, HLA-DP and HLA-DQ Alleles

| Compound Name | X₄-X₄′ | HLA-DR | HLA-DP | HLA-DQ | Total |
|---|---|---|---|---|---|
| desGla-FX ins[194] > [YDEDENQSPR]-HPC4 | QSPR-IVGG | 0 | 0 | 0 | 0 |
| desGla-FX ins[194] > [HTHHAPLSPR]-HPC4 | LSPR-IVGG | 0 | 0 | 0 | 0 |
| FX [191-194] > [NATLRPR]-HPC4 | LRPR-IVGG | 0 | 0 | 0 | 0 |
| FX [191-194] > [NATMRPR]-HPC4 | MRPR-IVGG | 0 | 0 | 0 | 0 |
| FX [191-194] > [NATMTPR]-HPC4 | MTPR-IVGG | 0 | 0 | 0 | 0 |
| FX [191-194] > [NATIQPR]-HPC4 | IQPR-IVGG | 0 | 0 | 0 | 0 |
| FX [191-194] > [NATIRPR]-HPC4 | IRPR-IVGG | 0 | 0 | 0 | 0 |
| FX [191-194] > [NATITPR]-HPC4 | ITPR-IVGG | 0 | 0 | 0 | 0 |
| FX [191-194] > [NATFRPR]-HPC4 | FRPR-IVGG | 0 | 0 | 0 | 0 |
| FX [191-194] > [NATLSPR]-HPC4 | LSPR-IVGG | 0 | 0 | 0 | 0 |
| FX [191-194] > [NATLQPR]-HPC4 | LQPR-IVGG | 0 | 0 | 0 | 0 |
| FX [191-194] > [NATLTPR]-HPC4 | LTPR-IVGG | 0 | 0 | 0 | 0 |
| FX [191-194] > [NATMQPR]-HPC4 | MQPR-IVGG | 0 | 0 | 0 | 0 |
| FX [191-194] > [NATIKPR]-HPC4 | IKPR-IVGG | 0 | 0 | 0 | 0 |
| FX [191-194] > [NATLEPR]-HPC4 | LEPR-IVGG | 0 | 0 | 0 | 0 |
| FX [191-194] > [NATDTPR]-HPC4 | DTPR-IVGG | 0 | 0 | 0 | 0 |
| FX [191-194] > [LTPR]-HPC4 | LTPR-IVGG | 0 | 0 | 0 | 0 |
| FX [191-194] > [MTPR]-HPC4 | MTPR-IVGG | 0 | 0 | 0 | 0 |
| FX [191-194] > [ITPR]-HPC4 | ITPR-IVGG | 0 | 0 | 0 | 0 |
| desGla-FX ins[194] > [NESTTKIKPR]-HPC4 | IKPR-IVGG | 0 | 0 | 0 | 0 |
| FX [191-194] > [FTPR]-HPC4 | FTPR-IVGG | 0 | 0 | 0 | 0 |
| FX [191-194] > [NATLKPR]-HPC4 | LKPR-IVGG | 0 | 0 | 0 | 0 |
| FX [191-194] > [NATFTPR]-HPC4 | FTPR-IVGG | 0 | 0 | 0 | 0 |
| FX [191-194] > [NATFKPR]-HPC4 | FKPR-IVGG | 0 | 0 | 0 | 0 |
| FX [191-194] > [NATMKPR]-HPC4 | MKPR-IVGG | 0 | 0 | 0 | 0 |
| FX [191-194] > [NATWQPR]-HPC4 | WQPR-IVGG | 0 | 0 | 0 | 0 |
| FX [191-194] > [NATLMPR]-HPC4 | LMPR-IVGG | 0 | 0 | 0.0171 | 0.0171 |
| desGla-FX ins[194] > [ATNATLDPR ]-HPC4 | LDPR-IVGG | 0.0297 | 0 | 0 | 0.0297 |
| FX [191-194] > [NATLDPR]-HPC4 | LDPR-IVGG | 0.0297 | 0 | 0 | 0.0297 |
| FX ins[194] > [PSILFKPR]-HPC4 | FKPR-IVGG | 0.0310 | 0.0015 | 0 | 0.0325 |
| FX [191-194] > [NATMMPR]-HPC4 | MMPR-IVGG | 0 | 0 | 0.0341 | 0.0341 |
| FX [191-194] > [NATIMPR]-HPC4 | IMPR-IVGG | 0 | 0 | 0.0341 | 0.0341 |
| FX ins[194] > [PSILMKPR]-HPC4 | MKPR-IVGG | 0.0544 | 0 | 0 | 0.0544 |
| FX ins[194] > [PSILWQPR]-HPC4 | WQPR-IVGG | 0.0377 | 0 | 0.0171 | 0.0548 |
| desGla-FX ins[194] > [DNSPSFIQIR]-HPC4 | IQIR-IVGG | 0.0631 | 0 | 0 | 0.0631 |
| FX ins[194] > [PSILLKPR]-HPC4 | LKPR-IVGG | 0.0676 | 0 | 0 | 0.0676 |
| desGla-FX ins[194] > [DFLAEGGGPR ]-HPC4 | GGPR-IVGG | 0 | 0 | 0.0802 | 0.0802 |
| FX ins[194] > [DFLAEGGGPR]-HPC4 | GGPR-IVGG | 0 | 0 | 0.0802 | 0.0802 |
| desGla-FX ins[194] > [DNEEGFFSAR]-HPC4 | FSAR-IVGG | 0 | 0 | 0.0891 | 0.0891 |
| desGla-FX ins[194] > [PDNI AWYLR]-HPC4 | WYLR-IVGG | 0.0426 | 0 | 0.0571 | 0.0997 |
| desGla-FX ins[194] > [LSKNNAIEPR]-HPC4 | IEPR-IVGG | 0.1150 | 0 | 0 | 0.1150 |
| FX ins[194] > [PSILFTPR]-HPC4 | FTPR-IVGG | 0.1159 | 0 | 0 | 0.1159 |
| FX ins[194] > [PSILLRPR]-HPC4 | LRPR-IVGG | 0.1166 | 0 | 0 | 0.1166 |
| FX ins[194] > [DFLAEGGGVR]-HPC4 | GGVR-IVGG | 0.0054 | 0 | 0.1122 | 0.1176 |
| desGla-FX ins[194] > [DFLAEGGGVR ]-HPC4 | GGVR-IVGG | 0.0054 | 0 | 0.1122 | 0.1176 |
| desGla-FX ins[194] > [TVELQGVVPR]-HPC4 | VVPR-IVGG | 0.0357 | 0 | 0.0854 | 0.1211 |
| desGla-FX ins[194] > [STPSILPAPR]-HPC4 | PAPR-IVGG | 0.2230 | 0 | 0 | 0.2230 |
| FX ins[194] > [PSILMTPR]-HPC4 | MTPR-IVGG | 0.2594 | 0 | 0.0171 | 0.2765 |
| FX ins[194] > [STPSILWQPR]-HPC4 | WQPR-IVGG | 0.5058 | 0 | 0.0171 | 0.5229 |
| FX ins[194] > [STPSILFKPR]-HPC4 | FKPR-IVGG | 0.5955 | 0 | 0 | 0.5955 |
| FX ins[194] > [STPSILFTPR]-HPC4 | FTPR-IVGG | 0.6102 | 0 | 0 | 0.6102 |
| FX ins[194] > [STPSILMKPR]-HPC4 | MKPR-IVGG | 0.6297 | 0 | 0 | 0.6297 |
| FX ins[194] > [STPSILLKPR]-HPC4 | LKPR-IVGG | 0.6623 | 0 | 0 | 0.6623 |
| FX ins[194] > [STPSILLRPR]-HPC4 | LRPR-IVGG | 0.6736 | 0 | 0 | 0.6736 |
| FX [191- | LDPR-IVGG | 0.0297 | 0 | 0.6657 | 0.6954 |
| 194] > [GGGSGGGSGDPKPSSEFEEFEIDEE | | | | | |
| EKGGGSGGGNATLDPR]-HPC4 | | | | | |
| desGla-FX ins[194] > [NRLAAALGIR]-HPC4 | LGIR-IVGG | 0.6979 | 0 | 0.0341 | 0.7320 |
| desGla-FX ins[194] > [SEYQTFFNPR]-HPC4 | FNPR-IVGG | 0.2268 | 0.4853 | 0.0439 | 0.7559 |
| FX ins[194] > [STPSI LMTPR]-HPC4 | MTPR-IVGG | 0.7526 | 0 | 0.0341 | 0.7868 |
| FX [191- | LDPR-IVGG | 0.0297 | 0.7269 | 0.2205 | 0.9770 |
| 194] > [GGGSGGGKEEEDIEFEEFESSPKPD | | | | | |
| GSGGGSGGGNATLDPR]-HPC4 | | | | | |

### Example 8

### Heparosan Conjugates—Quantification Method

The heparosan conjugates of the invention were analysed for purity by HPLC. HPLC was also used for conjugate quantifications. Quantifications were based on area under curve integration using the 280 nm wavelength absorption profile. Plasma derived human Factor X (Lot: HFX 1212, Molecular Innovations, Inc, Novi Mich., USA), was used as reference. A Zorbax 300SB-C3 column (4.6×50 mm; 3.5 μm Agilent, Cat. No.: 865973-909) was used. The column was operated on an Agilent 1100 Series HPLC furnished with fluorescence detector (Ex 280 nm, Em 348 nm). Column temperature was 30° C., with 5 μg sample injection and a flow rate of 1.5 ml/min. Column was eluted with a water (A)—acetonitrile (B) solvent system containing 0.1% trifluoroacetic acid. The gradient program was as follows: 0 min (25% B); 4 min (25% B); 14 min (46% B); 35 min (52% B); 40 min (90% B); 40.1 min (25% B).

### Example 9

### Heparosan Conjugates—SDS-PAGE Analysis

SDS PAGE analysis was performed using precast NuPage 7% tris-acetate gels, NuPage tris-acetate SDS running buffer and NuPage LDS sample buffer all from Invitrogen. Samples were denaturized (70° C. for 10 min.) before analysis. HiMark HMW (Invitrogen) was used as standard. Electrophoresis was run in an XCell Surelock Complete with power station (Invitrogen) for 80 min at 150 V, 120 mA. Gels were stained using SimplyBlue SafeStain from Invitrogen.

### Example 10

### Synthesis of Heparosan-Benzaldehyde Polymers

Functionalized HEP polymers of defined size are prepared by an enzymatic (PmHS1) polymerization reaction using the two sugar nucleotides UDP-GlcNAc and UDP-GlcUA. A priming trisaccharide (GlcUA-GlcNAc-GlcUA)N H₂ is used for initiating the reaction, and polymerization is run until depletion of sugar nucleotide building blocks. The terminal amine (originating from the primer) is then functionalized with a benzaldehyde functionality designed for reductive amination chemistry to GSC. Size of HEP polymers can be pre-determined by variation in sugar nucleotide: primer stoichiometry. The technique is described in detail in US 2010/0036001.

HEP-benzaldehydes can be prepared by reacting amine functionalized HEP polymers with a surplus of N-succinimidyl-4-formylbenzoic acid (Nano Letters (2007) 7(8), pp. 2207-2210) in aqueous neutral solution. The benzaldehyde functionalized polymers may be isolated by ion-exchange chromatography, size exclusion chromatography, or HPLC.

Terminal HEP amines may alternatively be functionalized into a maleimide reagent to facilitate coupling to cysteine in Factor X cysteine mutants. HEP-maleimides can be prepared by reacting amine functionalized HEP polymers with a surplus of N-maleimidobutyryl-oxysuccinimide ester (GMBS; Fujiwara, K., et al. (1988) J. Immunol. Meth. 112, 77-83).

The benzaldehyde functionalized polymers may be isolated by ion-exchange chromatography, size exclusion chromatography, or HPLC. Any HEP polymer functionalized with terminal primary amine functionality (HEP-NH₂) may be used in the present examples. Two options are shown below:

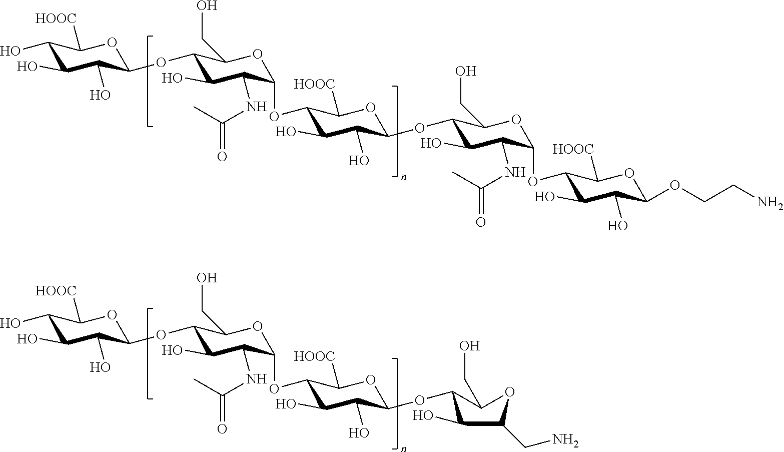

Furthermore the terminal sugar residue in the non-reducing end of the polysaccharide can be either N-acetylglucosamine or glucuronic acid (glucuronic acid is drawn above). Typically a mixture of both is to be expected if equimolar amounts of UDP-GlcNAc and UDP-GlcUA have been used in the polymerization reaction.

### Example 11

### Synthesis of 40 kDa Heparosan-GSC Reagent

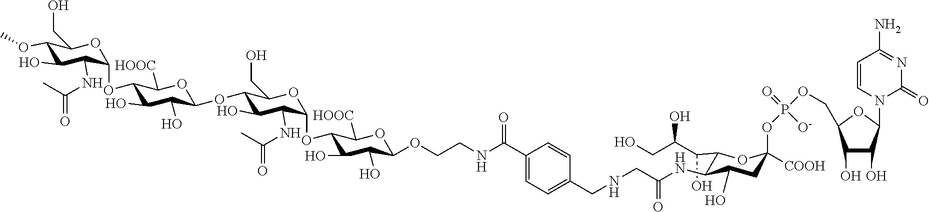

Glycyl sialic acid cytidine monophosphate (GSC) (20 mg; 32 μmol) in 5.0 ml 50 mM Hepes, 100 mM NaCl, 10 mM CaCl₂ buffer, pH 7.0 was added directly to dry 40 kDa HEP-benzaldehyde (99.7 mg; 2.5 μmol, nitrogen quantification). The mixture was gently rotated until all HEP-benzaldehyde had dissolved. During the following 2 hours, a 1M solution of sodium cyanoborohydride in MilliQ water was added in portions (5×50 μl), to reach a final concentration of 48 mM. Reaction mixture was left at room temperature overnight. Excess of GSC was then removed by dialysis as follows: the total reaction volume (5250 μl) was transferred to a dialysis cassette (Slide-A-Lyzer Dialysis Cassette, Thermo Scientific Prod#66810 with cut-off 10 kDa capacity: 3-12 ml). Solution was dialysed for 2 hours against 2000 ml of 25 mM Hepes buffer (pH 7.2) and once more for 17 h against 2000 ml of 25 mM Hepes buffer (pH 7.2). Complete removal of excess GSC from inner chamber was verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using GSC as reference. Inner chamber material was collected and freeze dried to give 83% (nitrogen quantification) 40 kDa HEP-GSC as white powder. The HEP-GSC reagent made by this procedure contains a HEP polymer attached to sialic acid cytidine monophosphate via a 4-methylbenzoyl linkage.

### Example 12

### Desilylation of pdFX

To plasma derived Factor X (14.3 mg) was added sialidase (Arthrobacter ureafaciens (AUS), 750 μl, 0.3 mg/ml, 200 U/ml) in 50 mM Hepes, 100 mM NaCl, pH 7.0 (10 ml), and left for 1 hour at room temperature. The reaction mixture was then diluted with 50 mM Hepes, 150 mM NaCl, pH 7.0 (5 ml), and cooled on ice. A solution of 100 mM EDTA (4 ml) was added in small portions. The EDTA treated sample was then applied to a 2×5 ml interconnected HiTrap Q FF ion-exchange columns (Amersham Biosciences, GE Healthcare) with a combined CV=10 ml and equilibrated with 50 mM Hepes, 150 mM NaCl, 0.01% Tween 80, pH 7.0. Sialidase was eluted with 50 mM Hepes, 150 mM NaCl, 0.01% Tween 80, pH 7.0 (4 CV), before eluting asialo-pdFX with 50 mM Hepes, 150 mM NaCl, 10 mM CaCl₂, 0.01% Tween 80, pH 7.0 (10 CV). Asialo-pdFX was in this way isolated in 50 mM Hepes, 150 mM NaCl, 10 mM CaCl₂, 0.01% Tween 80, pH 7.0 (19 ml). Yield (13.1 mg) and concentration (0.69 mg/ml) was determined by HPLC.

### Example 13

### Synthesis of 40 kDa Heparosant-[N]-pdFX

To asialo-pdFX (13.1 mg) in 50 mM Hepes, 150 mM NaCl, 10 mM CaCl₂, 0.01% Tween 80, pH 7.0 (19 ml) was added 40 kDa HEP-GSC (19.4 mg) and rat ST3GaIIII enzyme (2.44 mg; 1.1 unit/mg) in 20 mM Hepes, 120 mM NaCl, 50% glycerol, pH 7.0 (4.9 ml). The reaction mixture was incubated for 16 hours at 32° C. under slow stirring. A solution of 157 mM CMP-NAN in 50 mM Hepes, 150 mM NaCl, 10 mM CaCl2, pH 7.0 (0.71 ml) was then added, and the reaction was incubated at 32° C. for an additional hour. HPLC analysis showed a product distribution containing un-reacted pdFX (56%), mono HEPylated pdFX (37%) and polyHEPylated product (7%). The reaction mixture was divided into 4 portions, and each portion was applied to a HiLoad 16/60 Superdex200 pregrade column (CV=124 ml), equilibrated with 50 mM Hepes, 150 mM NaCl, 10 mM CaCl₂, 0.01% Tween 80, pH 7.0. The column was eluted with the same buffer and fractions containing un-reacted and HEP modified pdFX from all runs were pooled into a single 48 ml fraction. Fractions were cooled on ice, and 100 mM EDTA solution (7 ml) was added in small portions. The EDTA treated sample was then applied to a 2×5 ml HiTrap Q FF ion-exchange column (Amersham Biosciences, GE Healthcare) with a combined CV=10 ml and equilibrated with 10 mM His, 100 mM NaCl, 0.01% Tween 80, pH 7.5. The column was washed with 4 column volumes of 10 mM His, 100 mM NaCl, pH 7.5 and 10 column volumes of 10 mM His, 100 mM NaCl, 10 mM CaCl₂, 0.01% Tween 80, pH 7.5 to eluted unmodified pdFX. The pH was then lowered to 6.0 with 10 mM His, 100 mM NaCl, 10 mM CaCl₂, 0.01% Tween 80, pH 6.0 (10 column volumes). HEPylated pdFX was then eluted off the column with 10 column volumes of 10 mM His, 100 mM NaCl, 10 mM CaCl₂, 0.01% Tween 80, pH 6.0 (40%) and 10 mM His, 1 M NaCl, 10 mM CaCl₂, 0.01% Tween 80, pH 6.0 (60%) buffer mixture. Combined fractions were then dialyzed against 10 mM His, 150 mM NaCl, 5 mM CaCl₂, 0.005% Tween 80, pH 6.4 using a Slide-A-Lyzer cassette (Thermo Scientific) with a cut-off of 10 kDa. Yield (2 mg) and concentration (0.45 mg/ml) was determined by HPLC.

### Example 14

### Conjugation of Heparosan Polymers to the N-Glycans of Thrombin Sensitive FX Molecules

Factor X molecules carrying modifications for example in the activation peptide as described herein, may be conjugated to HEParosan in a similar manner as described in Examples 12-13. To facilitate N-glycan conjugation, the FX molecule is initially treated with sialidase as described in example 12. The process removes sialic acids from the N-glycan termini and allows for sialyltransferase mediated transfer of heparosan modified sialic acids from the HEP-GSC reagents to the asialo-FX molecule. After capping of non-reacted N-glycan termini with sialic acid the HEP-FX molecules are isolated by a size exclusion chromatography, anion/cation exchange chromatography, affinity chromatography or a combination of these chromatographic methods.

### Example 15

### Selective Reduction of a Factor X Single Cysteine Mutant

Factor X single cysteine molecules when produced in mammalian cells are typically isolated with its non-paired cysteine blocked by low molecular thiols as mixed disulfides. To facilitate HEP conjugation, the mixed disulfide initially needs to be unblocked in order to make the thiol group available for coupling. Unblocking can be performed by chemical reduction using phosphine-based reducing reagents. Alternatively, Factor X single cysteine molecules can be reduced using a glutathione based redox buffer system, in similar manner as described for FVIIa407C in US 20090041744. In one method, non-reduced Factor X single cysteine molecules are incubated for 24 hours at room temperature in a mixture of containing GSH, GSSG, and Grx2. The reduced Factor X single cysteine molecule is then isolated by ion-exchange chromatography as described in example 12.

### Example 16

### Synthesis of 40 kDa HEP-[C]-FX Cysteine Molecule

A solution of single cysteine reduced Factor X molecules as prepared in the above Example 15 is reacted with a 41.5 kDa HEP maleimide reagent in an appropriate buffer such as 50 mM Hepes, 100 mM NaCl, 10 mM CaCl₂, pH 7.0. The conjugation process can be followed by HPLC methods as described in Example 8. When conjugation is complete, the HEP-[C]-FX cysteine molecule can be isolated by a size exclusion chromatography, anion/cation exchange chromatography, affinity chromatography or a combination of these chromatographic methods, as described in Example 13.

### Example 17

### GlycoHEPylation of Human Factor X Increase the Circulatory Half-Life of Factor X in Haemophilia a Mice

A pharmacokinetic study of human plasma derived Factor X (pdFX) and a human pdFX that was glycoHEPylated on the N-glycans with 40 kDa heparosan (40 kDa HEP), hereafter referred to as 40 kDa HEP-[N]-pdFX, was performed in FVIII knock-out (FVIII-KO) mice. The objective of the study was to evaluate the effect of protraction on the pharmacokinetics of pdFX. The compounds investigated were pdFX purchased from Molecular Innovations, Inc (Novi Mich., USA); catalog no HCX-0050 Lot. HFX-1212. Based on this material, a glycoHEPylation was performed based on the methods outlined in Examples 12-13 to produce a 40 kDa HEP-[N]-pdFX. A total of 30 FVIII-KO mice of mixed gender, bred at Taconic, were included in the study. They were dosed with a single bolus IV injection of 16.7 nmol/kg equal to 1 mg/kg. The dose volume was 5 ml/kg. Blood samples were collected from the orbital sinus for 18 (pdFX) and 96 (40 kDa HEP-[N]-pdFX) hours post dosing in a sparse sampling schedule (3 mice per time point, 3 samples per mice). The plasma levels of Factor X were measured using a modified commercial Factor X enzyme immunoassay (Human FX ELISA kit, cat no. KSP-134, Nordic BioSite, Copenhagen, Denmark) where Factor X is detected in a monoclonal anti-FX coated plate with a polyclonal anti-FX-biotin and a streptavidin-peroxidase conjugate. The calibrator provided by the kit was exchanged with pdFX and 40 kDa HEP-[N]-pdFX spiked into diluted FVIII KO mouse plasma for analysis of plasma levels of pdFX and HEP-pdFX respectively. QC samples were prepared by spiking pdFX or HEP-pdFX into diluted F8-KO mouse plasma.

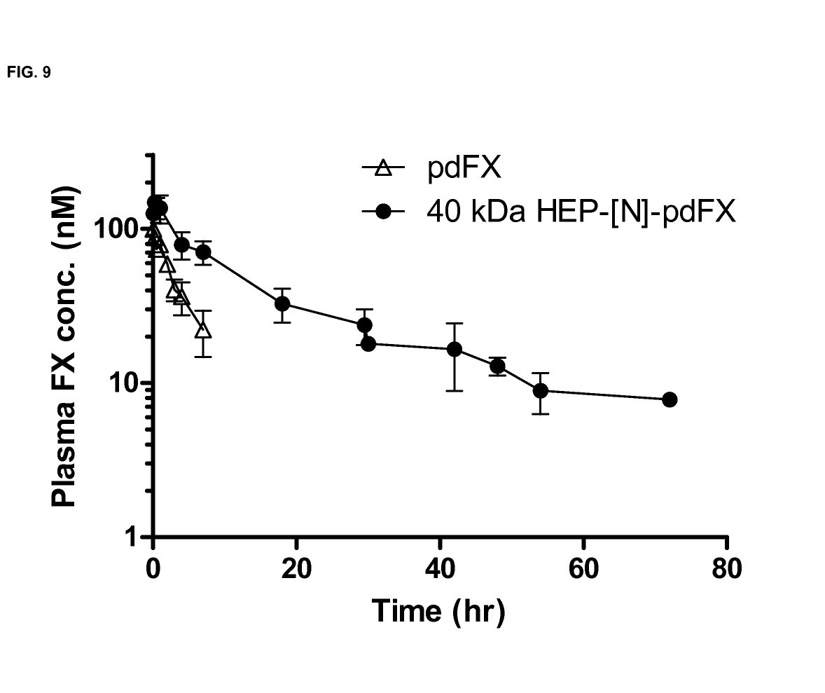

The pharmacokinetic parameters were calculated by non-compartmental analysis (NCA). The plasma profile and pharmacokinetic parameters are shown in FIG. 9 and Table 9, respectively.

The plasma half-life (T½) and mean residence time (MRT) of pdFX in the FVIII-KO mice were 3.8 and 5.2 hours, respectively. By conjugating a 40 kDa heparosan polymer to Factor X the half-life and MRT increased by a factor of 5 to 19.5 and 24.7 hours, respectively. As described below in Example 18, glycoPEGylation of human Factor X showed a 4.4-fold prolonged plasma half-life in C57BL6 mice compared to non-modified human FX. The plasma profiles for Factor X conjugated to HEP and PEG, respectively, were comparable (cf. FIG. 9).

TABLE 9
Mean pharmacokinetic parameters of FX and 40 kDa HEP-[N]-pdFX
after IV administration of 1 mg/kg to FVIII-KO mice

| | Cmax | AUC | Vz | Cl | MRT | T½ |
|---|---|---|---|---|---|---|
| Compound | (nmol/L) | (hr*nmol/L) | (mL/kg) | (mL/hr/kg) | (hr) | (hr) |
| pdFX | 99 | 443* | 205 | 37.7 | 5.2 | 3.8 |
| 40 kDa HEP-[N]- | 148 | 2257 | 209 | 7.4 | 24.7 | 19.5 |
| pdFX | | | | | | |

### Example 18

### Prolonged Circulatory Plasma Half-Life of GlycoPEGylated Human Factor X in Normal Mice

A pharmacokinetic study of human FX and a PEGylated human FX (FX-GP) was performed, in nave mice. The objective of the study was to investigate the effect of protraction of Factor X (in this case a glycopegylation) on the clearance of the compound. The compounds investigated were Factor X (HCX-0050 Lot. AA1208) and FX-GP.

Wild type, plasma-derived Factor X was purchased from Haematologic Technologies (HCX-0050). Based on this material, a glycoPEGylation was performed according to standard procedures used previously at Novo Nordisk for FVIIa and FIX (Neose protocol). The PEGylation and subsequent chromatographic separation gave a preparation of mono-PEGylated devoid of non-PEGylated FX but containing approx 5% of di-PEGylated FX. The site(s) of PEGylation was not determined.

A total of 30 C57BL/6J mice bred at Taconic were dosed with a single bolus IV injection of the compounds. The plasma levels of FX were measured by an enzyme immunoassay (EIA) for 168 hours post dosing by sparse sampling (3 mice per time point, 3 samples per mice, see section 5.1.4.1). The pharmacokinetic parameters were calculated by non-compartmental analysis (NCA). The measured plasma concentrations are presented in Table 10 and obtained PK parameters are shown in Table 11.

TABLE 10
Plasma Concentration at the Time of Observation and Mean Concentrations of FX and
FX-GP in Naïve Mice After Single Dose I.V. Administration of 1 mg/kg

| | | TIME (hr) | | | | | | | |
|---|---|---|---|---|---|---|---|---|---|
| | | 0.080 | 0.25 | 0.50 | 1.0 | 4.0 | 7.0 | 17 | 24 |
| COMPOUND | SUBJECT | Concentration (ug/ml) | | | | | | | |
| FX | 1.0 | 8.6 | | | | | | <LLOQ | |
| | 2.0 | 11 | | | | | | <LLOQ | |
| | 3.0 | 11 | | | | | | <LLOQ | |
| | 4.0 | | 8.4 | | | | 0.89 | | |
| | 5.0 | | 11 | | | | 1.0 | | |
| | 6.0 | | 12 | | | | 1.3 | | |
| | 7.0 | | | 5.8 | | | | | <LLOQ |
| | 8.0 | | | 9.3 | | | | | <LLOQ |
| | 9.0 | | | 12 | | | | | <LLOQ |
| | 10 | | | | 3.8 | | | | |
| | 11 | | | | 6.1 | | | | |
| | 12 | | | | 6.3 | | | | |
| | 13 | | | | | 2.2 | | | |
| | 14 | | | | | 2.3 | | | |
| | 15 | | | | | 3.3 | | | |
| | Mean | 10.123 | 10.273 | 9.083 | 5.387 | 2.597 | 1.070 | NC | NC |
| | SD | 1.423 | 1.716 | 3.233 | 1.394 | 0.637 | 0.203 | NC | NC |
| | CV % | 14.1 | 16.7 | 35.6 | 25.9 | 24.5 | 19.0 | NC | NC |
| FX-GP | 16 | 6.6 | | | | | | 1.7 | |
| | 17 | 8.3 | | | | | | 2.8 | |
| | 18 | 8.3 | | | | | | 1.8 | |
| | 19 | | 7.2 | | | | 4.5 | | |
| | 20 | | 5.8 | | | | 3.1 | | |
| | 21 | | 6.9 | | | | 4.4 | | |
| | 22 | | | 4.4 | | | | | 1.1 |
| | 23 | | | 5.2 | | | | | 0.73 |
| | 24 | | | 6.9 | | | | | 2.1 |
| | 25 | | | | 2.4 | | | | |
| | 26 | | | | 5.0 | | | | |
| | 27 | | | | 2.8 | | | | |
| | 28 | | | | | 5.3 | | | |
| | 29 | | | | | 7.0 | | | |
| | 30 | | | | | 6.8 | | | |
| | Mean | 7.697 | 6.613 | 5.517 | 3.420 | 6.353 | 3.977 | 2.130 | 1.323 |
| | SD | 0.984 | 0.755 | 1.294 | 1.391 | 0.944 | 0.806 | 0.618 | 0.703 |
| | CV % | 12.8 | 11.4 | 23.5 | 40.7 | 14.9 | 20.3 | 29.0 | 53.1 |

| | | TIME (hr) | | | | | | |
|---|---|---|---|---|---|---|---|---|
| | | 30 | 48 | 72 | 96 | 120 | 140 | 170 |
| COMPOUND | SUBJECT | Concentration (ug/ml) | | | | | | |
| FX | 1.0 | | | <LLOQ | | | | |
| | 2.0 | | | <LLOQ | | | | |
| | 3.0 | | | <LLOQ | | | | |
| | 4.0 | | | | <LLOQ | | | |
| | 5.0 | | | | <LLOQ | | | |
| | 6.0 | | | | <LLOQ | | | |
| | 7.0 | | | | | <LLOQ | | |
| | 8.0 | | | | | <LLOQ | | |
| | 9.0 | | | | | <LLOQ | | |
| | 10 | <LLOQ | | | | | <LLOQ | |
| | 11 | <LLOQ | | | | | <LLOQ | |
| | 12 | <LLOQ | | | | | <LLOQ | |
| | 13 | | <LLOQ | | | | | <LLOQ |
| | 14 | | <LLOQ | | | | | <LLOQ |
| | 15 | | <LLOQ | | | | | <LLOQ |
| | Mean | NC | NC | NC | NC | NC | NC | NC |
| | SD | NC | NC | NC | NC | NC | NC | NC |
| | CV % | NC | NC | NC | NC | NC | NC | NC |
| FX-GP | 16 | | | 0.73 | | | | |
| | 17 | | | 0.97 | | | | |
| | 18 | | | <LLOQ | | | | |
| | 19 | | | | <LLOQ | | | |
| | 20 | | | | <LLOQ | | | |
| | 21 | | | | <LLOQ | | | |
| | 22 | | | | | <LLOQ | | |
| | 23 | | | | | <LLOQ | | |
| | 24 | | | | | <LLOQ | | |
| | 25 | 0.66 | | | | | <LLOQ | |
| | 26 | 0.47 | | | | | <LLOQ | |
| | 27 | 0.48 | | | | | <LLOQ | |
| | 28 | | <LLOQ | | | | | |
| | 29 | | <LLOQ | | | | | <LLOQ |
| | 30 | | <LLOQ | | | | | <LLOQ |
| | Mean | 0.537 | NC | 0.850 | NC | NC | NC | NC |
| | SD | 0.107 | NC | 0.170 | NC | NC | NC | NC |
| | CV % | 19.9 | NC | 20.0 | NC | NC | NC | NC |

TABLE 11
Estimated Pharmacokinetic Parameters After I.V.
Administration of FX and FX-GP Based on Sparse
Sampling and Non-Compartmental Analysis

| Com- | Dose | t½ | CL | AUC | AUC % Extrap | MRT |
|---|---|---|---|---|---|---|
| pound | (mg/kg) | (hr) | (ml/hr/kg) | (hr*ug/ml) | (%) | (hr) |
| FX | 1 | 2.3 | 34 | 30 | 12 | 3.0 |
| FX-GP | 1 | 9.9 | 11 | 94 | 11 | 14 |

GlycoPEGylation of human pdFX through conjugation to the N-linked glycans located in the activation peptide of pdFX showed a 4.4-fold prolonged plasma half-life in mice compared to non-modified human pdFX.

### Example 19

### Screening the Thrombin Sensitive FX Molecules to Evaluate the Kinetic Rate Constants for Activation by α-Thrombin (FIIa)

### A. Assay Protocol

The reaction kinetics describing the activation of thrombin sensitive FX molecules by human α-thrombin (FIIa) were evaluated using a classical Michaelis Menten approach in which a range of Factor X or thrombin sensitive FX molecules was used to calculate the kinetic rate constants, where the substrate (thrombin sensitive FX molecules) was at least 10 to 20 fold in excess of the activating protease (FIIa). This method was carried out essentially as described by Louvain-Quintard et al. (2005) JBC, 280: 41352-41359 with minor modifications in the protocol to accommodate screening multiple thrombin sensitive FX molecules concurrently. Briefly, thrombin sensitive FX molecules were diluted in assay buffer (50 mM Hepes, 150 mM NaCl, 10 mM CaCl₂, 0.1% PEG8000, 0.1% BSA, pH 7.4) to an initial working concentration of ˜2 to 4 μM, representing the highest concentration of thrombin sensitive FX molecule tested. The thrombin sensitive FX molecules were further serially diluted 2-fold into assay buffer to generate a dose response curve ranging from 0 nM to 4000 nM in a 96-well polypropylene assay plate. In some cases, stock concentrations only permitted dose response curves ranging from 0 to 1000 nM, 0 to 2000 nM or other final concentration interval between 1000 nM and 4000 nM. Thrombin activation reactions were triggered by the addition of 11 μL of 10 nM α-thrombin diluted in assay buffer to 100 μL of the thrombin sensitive FX for a final α-thrombin concentration of 1 nM in a 111 μL reaction volume. Reactions were incubated at 37° C. for a total of 30 min, 60 min or 120 min depending on the expected reaction rate. Reactions were quenched at the end of the incubation period by withdrawing duplicate 40 μL aliquots and adding to each of two wells in a black 96-well polystyrene assay plate containing 10 μL of 500 nM hirudin (recombinant His-tagged) yielding a final concentration of 100 nM hirudin. The quantity of FXa generated during that assay was determined by adding 50 μL of a 1 mM solution of a specific fluorogenic FXa substrate, Pefafluor FXa (CH₃SO₂-D-CHA-Gly-Arg-AMC; Pentapharm, Switzerland), and by comparison to a standard curve of known amounts of FXa (0 nM to 5 nM). The final concentration of Pefafluor FXa in the quantitation reaction was 0.5 mM.

Reaction progress curves were monitored in a SpectraMax fluorescence plate reader for 10 min at 25° C. and analysed as described below. The catalytically active concentration of the FXa standard was determined by titration with 4-Methylumbelliferyl 4-guanidinobenzoate (MUGB), a fluorogenic ester substrate developed as an active site titrant for serine proteases essentially as described by Payne et al. (1996) Biochemistry, 35(22): 7100-7106. Due to the sensitivity of the assay, in some instances it was necessary to inhibit trace amounts of background FXa activity in the sample by inhibition with 100 μM Glu-Gly-Arg-chloromethylketone (EGR-cmk) for 2 hrs at room temperature followed by extensive dialysis against a storage buffer comprising 10 mM MES, 150 mM NaCl, 10 mM CaCl₂, pH 6.0.

### B. Data Analysis

The raw reaction progress curves of Pefafluor FXa hydrolysis were analysed to determine the kinetic parameters k_{cat} (s⁻¹), K_{M} (nM or M) and k_{cat}/K_{M} (M⁻¹s⁻¹). Raw reaction velocities were initially analysed as fluorescence units/s (FU/s) within the Softmax Pro software suite associated with the SpectraMax fluorescence plate reader and subsequently converted to nM FXa using a standard curve created from the reaction velocities (FU/s) of know amounts of FXa (see above). The concentration of FXa generated during the course of the assay was then transformed into reaction velocities of the form nM FXa/s using equation (1).

$$\frac{nM FXa}{36 C .} = \frac{nM FXa Generated}{Total Reaction Time ( s )} \qquad Equation ( 1 )$$

Reaction velocities (nM FXa/s) were plotted against the concentrations of thrombin sensitive FX molecules and fit to the function of a standard rectangular hyperbola (i.e. Michaelis Menten equation) given by equation (2) to yield the fit values of k_{cat} and K_{M}, where E is the concentration of activating protease (FIIa) and Sₒ is the concentration of thrombin sensitive FX molecule in the dose response curve.

$$Reaction Velocity ( nM FXa \text{/} s ) = \frac{{k}_{cat} E [ {S}_{o} ]}{{K}_{M} + [ {S}_{o} ]} \qquad Equation ( 2 )$$

The specificity constant k_{cat}/K_{M} was calculated directly from the fit values of K_{M} and k_{cat} upon evaluation of equation (2).

For reactions that resulted in an apparent K_{M} that was indeterminate or greater than the highest tested concentration of thrombin sensitive FX molecule in the assay, the data was analysed in the linear dose range of the assay. For data collected using the linear range of the assay, the k_{cat}/K_{M} kinetic constants were directly calculated from the slopes of linear regression analyses of the thrombin sensitive FX concentrations versus the velocity of FXa generation (FXa/s) according to equation (3).

$$\frac{{k}_{cat}}{{K}_{M}} = \frac{slope}{[ FIIa ]} \qquad Equation ( 3 )$$

### C. Results

Tables 12-13 below set forth the kinetic parameters (k_{cat}, K_{M} and k_{cat}/K_{M}) determined for the activation of HPC4-tagged thrombin sensitive FX molecules by α-thrombin (FIIa), as well as recombinant FX (designated as FX-HPC4) and plasma purified FX (Molecular Innovations, Novi Mich., USA). Tables 12 and 13 also provide the standard deviation (S.D.) and the number of assays performed (n). Data are presented in Tables 12 and 13 as the ranked k_{cat}/K_{M} values.

The observed specificity constants (k_{cat}/K_{M}) ranged from no detectable activation by thrombin (designated No Activity) to k_{cat}/K_{M} values of 2.8E+04 M⁻¹s⁻¹ for a variant with a modified fibrinopeptide A (FpA) sequence that has a proline at X₂ (FX ins[194]>[DFLAEGGGPR]-HPC4). This activation rate is 10× the observed activation rate for a variant having the unmodified FpA sequence (FX ins[194]>[DFLAEGGGVR]-HPC4). Despite having the same engineered cleavage site, the method of introducing the cleavage site into the FX molecule significantly affected the activation rate. For instance, the two thrombin sensitive FX molecules, FX [191-194]>[MTPR]-HPC4 and FX [191-194]>[NATMTPR]-HPC4 comprise the X₄-X_{4′} cleavage sequence of MTPR-IVGG, wherein the cleavage occurs between X₁ and X_{1′} (i.e. R—I bond). The FX [191-194]>[MTPR]-HPC4 molecule is readily activated at a rate of 1.4E+03 M⁻¹s⁻¹ and the FX [191-194]>[NATMTPR]-HPC4 molecule cannot be activated (see Tables 12 and 13). While many of the preferred thrombin sensitive FX molecules show favourable activation kinetics with similar k_{cat}/K_{M} values in the range of 1.0E+03 to 3.0E+03 M⁻¹s⁻¹, the aforementioned thrombin sensitive FX molecules are differentiated by variances in the individual k_{cat} and K_{M} values (Table 12: For instance compare FX [191-194]>[MTPR]-HPC4 having a lower K_{M} of 1129 nM with FX ins[194]>[DFLAEGGGVR]-HPC4 having a K_{M} of 2239 nM or FX [191-194]>[LTPR]-HPC4 having a K_{M} of 2703 nM, each of which have 5-10 fold higher k_{cat} values than FX [191-194]>[MTPR]-HPC4.

Table 14 sets forth the kinetic parameters (k_{cat}, K_{M} and k_{cat}/K_{M}) determined for the activation of the thrombin sensitive FX molecule FX ins[194]>[DFLAEGGGVR]-HPC4, which has been conjugated with a 21 kDa, 40 kDa or 73 kDa heparosan polymer for mono-hepylation on a N-glycan in the activation peptide of the molecule. As shown in Table 14, there is no significant effect of hepylation on the observed kinetic parameters for the tested molecule.

TABLE 12
Kinetic Parameters for Activation of Thrombin Sensitive FX Molecules (Hyperbolic)

| | | k_{cat} | ±S.D. | K_{M} | ±S.D. | k_{cat}/K_{M} | ±S.D. | |
|---|---|---|---|---|---|---|---|---|
| Compound Name | X₄-X_{4′} | (s⁻¹) | (s⁻¹) | (nM) | (nM) | (M⁻¹s⁻¹) | (M⁻¹s⁻¹) | n |
| FX ins[194]>[DFLAEGGGPR]-HPC4 | GGPR-IVGG | 1.6E−02 | 1.0E−02 | 592 | 417 | 2.8E+04 | 2.2E+03 | 2 |
| desGla-FX ins[194]>[DFLAEGGGPR]-HPC4 | GGPR-IVGG | 8.8E−03 | 7.8E−03 | 1296 | 964 | 6.4E+03 | 1.3E+03 | 2 |
| FX ins[194]>[STPSILFKPR]-HPC4 | FKPR-IVGG | 7.0E−03 | 1.2E−03 | 1623 | 554 | 4.7E+03 | 2.3E+03 | 2 |
| FX [191-194]>[LTPR]-HPC4 | LTPR-IVGG | 9.0E−03 | 1.1E−03 | 2703 | 317 | 3.4E+03 | 7.9E+02 | 2 |
| FX ins[194]>[PSILFTPR]-HPC4 | FTPR-IVGG | 6.8E−03 | n.d. | 2049 | n.d. | 3.3E+03 | n.d. | 1 |
| FX ins[194]>[DFLAEGGGVR]-HPC4 | GGVR-IVGG | 6.0E−03 | 3.9E−03 | 2239 | 632 | 2.6E+03 | 1.2E+03 | 3 |
| FX ins[194]>[STPSILLKPR]-HPC4 | LKPR-IVGG | 3.0E−03 | 1.9E−03 | 1435 | 800 | 2.0E+03 | 1.9E+02 | 3 |
| FX [191-194]>[MTPR]-HPC4 | MTPR-IVGG | 1.4E−03 | 5.3E−04 | 1129 | 741 | 1.4E+03 | 4.9E+02 | 4 |
| FX [191-194]>[LTPR]-HPC4 | LTPR-IVGG | 1.8E−04 | 1.1E−06 | 141 | 8 | 1.2E+03 | 8.1E+01 | 2 |
| FX [191-194]>[NATLTPR]-HPC4 | LTPR-IVGG | 2.2E−03 | 2.0E−04 | 2328 | 600 | 1.0E+03 | 1.9E+02 | 3 |
| FX [191-194]>[NATLQPR]-HPC4 | LQPR-IVGG | 2.7E−03 | 2.4E−03 | 2669 | 146 | 9.9E+02 | 8.4E+02 | 2 |
| FX ins[194]>[STPSILFTPR]-HPC4 | FTPR-IVGG | 4.8E−03 | n.d. | 6013 | n.d. | 7.9E+02 | n.d. | 1 |
| desGla-FX ins[194]>[STPSILPAPR]-HPC4 | PAPR-IVGG | 1.1E−03 | n.d. | 1517 | n.d. | 7.2E+02 | n.d. | 1 |
| FX [191-194]>[NATLKPR]-HPC4 | LKPR-IVGG | 1.6E−03 | n.d. | 2458 | n.d. | 6.5E+02 | n.d. | 1 |
| FX [191-194]>[NATFKPR]-HPC4 | FKPR-IVGG | 6.3E−04 | n.d. | 1327 | n.d. | 4.8E+02 | n.d. | 1 |
| desGla-FX ins[194]>[NESTTKIKPR]-HPC4 | IKPR-IVGG | 1.0E−03 | 3.5E−05 | 2397 | 91 | 4.4E+02 | 3.1E+01 | 2 |
| desGla-FX ins[194]>[SEYQTFFNPR]-HPC4 | FNPR-IVGG | 1.2E−03 | 4.4E−04 | 2702 | 1086 | 4.3E+02 | 3.2E+01 | 3 |
| FX [191-194]>[NATMQPR]-HPC4 | MQPR-IVGG | 7.0E−04 | 8.1E−04 | 1597 | 1174 | 3.8E+02 | 1.5E+02 | 4 |
| FX [191-194]>[NATMKPR]-HPC4 | MKPR-IVGG | 1.8E−04 | 4.0E−05 | 674 | 103 | 2.8E+02 | 9.9E+01 | 3 |
| FX [191-194]>[NATLSPR]-HPC4 | LSPR-IVGG | 6.5E−05 | 5.4E−06 | 264 | 33 | 2.5E+02 | 1.1E+01 | 2 |
| FX [191-194]>[NATWQPR]-HPC4 | WQPR-IVGG | 6.6E−04 | 6.1E−05 | 4167 | 996 | 1.6E+02 | 4.3E+01 | 3 |
| FX [191-194]>[NATLDPR]-HPC4 | LDPR-IVGG | 1.6E−04 | 2.9E−05 | 1151 | 228 | 1.4E+02 | 2.3E+00 | 2 |

TABLE 13
Kinetic Parameters for Activation of Thrombin Sensitive FX Molecules (Linear)

| | | k_{cat}/K_{M} | ±S.D. | |
|---|---|---|---|---|
| Compound Name | X₄-X_{4′} | (M⁻¹s⁻¹) | (M⁻¹s⁻¹) | n |
| FX ins[194] > [DFLAEGGGPR]-HPC4 | GGPR-IVGG | 1.3E+04 | n.d. | 1 |
| FX ins[194] > [PSILFTPR]-HPC4 | FTPR-IVGG | 8.5E+03 | n.d. | 1 |
| desGla-FX ins[194] > [DFLAEGGGPR ]-HPC4 | GGPR-IVGG | 5.5E+03 | n.d. | 1 |
| FX [191-194] > [LTPR]-HPC4 | LTPR-IVGG | 2.1E+03 | 6.0E+02 | 2 |
| FX ins[194] > [STPSILFKPR]-HPC4 | FKPR-IVGG | 1.9E+03 | 1.0E+02 | 2 |
| FX ins[194] > [DFLAEGGGVR]-HPC4 | GGVR-IVGG | 1.6E+03 | 4.5E+02 | 6 |
| FX ins[194] > [STPSILLKPR]-HPC4 | LKPR-IVGG | 1.2E+03 | 4.3E+02 | 4 |
| FX ins[194] > [PSILMTPR]-HPC4 | MTPR-IVGG | 8.6E+02 | n.d. | 1 |
| FX ins[194] > [STPSILMTPR]-HPC4 | MTPR-IVGG | 7.9E+02 | n.d. | 1 |
| FX ins[194] > [PSILLKPR]-HPC4 | LKPR-IVGG | 7.5E+02 | n.d. | 1 |
| FX ins[194] > [STPSILFTPR]-HPC4 | FTPR-IVGG | 7.1E+02 | n.d. | 1 |
| FX [191- | LDPR-IVGG | 6.2E+02 | 3.0E+02 | 2 |
| 194] > [GGGSGGGKEEEDIEFEEFESSPKPD | | | | |
| GSGGGSGGGNATLDPR]-HPC4 | | | | |
| FX [191-194] > [NATLDPR]-HPC4 | LDPR-IVGG | 6.1E+02 | n.d. | 1 |
| FX [191-194] > [NATITPR]-HPC4 | ITPR-IVGG | 4.6E+02 | 4.5E+01 | 3 |
| desGla-FX ins[194] > [HTHHAPLSPR]-HPC4 | LSPR-IVGG | 4.4E+02 | 5.8E+01 | 3 |
| FX [191-194] > [NATLKPR]-HPC4 | LKPR-IVGG | 3.7E+02 | 1.4E+02 | 4 |
| FX [191- | LDPR-IVGG | 3.5E+02 | 9.0E+01 | 3 |
| 194] > [GGGSGGGSGDPKPSSEFEEFEIDEE | | | | |
| EKGGGSGGGNATLDPR]-HPC4 | | | | |
| FX [191-194] > [NATFKPR]-HPC4 | FKPR-IVGG | 3.2E+02 | 1.1E+02 | 3 |
| desGla-FX ins[194] > [SEYQTFFNPR]-HPC4 | FNPR-IVGG | 2.8E+02 | n.d. | 1 |
| FX [191-194] > [NATLQPR]-HPC4 | LQPR-IVGG | 2.7E+02 | n.d. | 1 |
| desGla-FX ins[194] > [NESTTKIKPR]-HPC4 | IKPR-IVGG | 2.6E+02 | n.d. | 1 |
| FX [191-194] > [NATMQPR]-HPC4 | MQPR-IVGG | 1.3E+02 | 9.3E+01 | 2 |
| FX [191-194] > [NATWQPR]-HPC4 | WQPR-IVGG | 1.3E+02 | n.d. | 1 |
| desGla-FX ins[194] > [TVELQGVVPR]-HPC4 | VVPR-IVGG | 1.1E+02 | 3.0E+01 | 3 |
| desGla-FX ins[194] > [DNEEGFFSAR]-HPC4 | FSAR-IVGG | 3.6E+01 | 2.7E+00 | 3 |
| FX [191-194] > [NATIQPR]-HPC4 | IQPR-IVGG | 2.2E+01 | n.d. | 1 |
| FX [191-194] > [NATLMPR]-HPC4 | LMPR-IVGG | No Activity | n.d. | 2 |
| FX [191-194] > [NATLRPR]-HPC4 | LRPR-IVGG | No Activity | n.d. | 2 |
| FX [191-194] > [NATMMPR]-HPC4 | MMPR-IVGG | No Activity | n.d. | 1 |
| FX [191-194] > [NATMTPR]-HPC4 | MTPR-IVGG | No Activity | n.d. | 2 |
| FX [191-194] > [NATIRPR]-HPC4 | IRPR-IVGG | No Activity | n.d. | 2 |
| FX [191-194] > [NATIKPR]-HPC4 | IKPR-IVGG | No Activity | n.d. | 2 |
| FX [191-194] > [NATLEPR]-HPC4 | LEPR-IVGG | No Activity | n.d. | 1 |
| FX [191-194] > [NATDTPR]-HPC4 | DTPR-IVGG | No Activity | n.d. | 2 |
| FX-HPC4 | NLTR-IVGG | No Activity | n.d. | 3 |

TABLE 14
Kinetic Parameters for Activation of Thrombin Sensitive FX Molecules
Glyco-Conjugated with Heparosan (21, 40 and 73 kDa HEP)

| | | k_{cat} | ±S.D. | K_{M} | ±S.D. | k_{cat}/K_{M} | ±S.D. | |
|---|---|---|---|---|---|---|---|---|
| Compound Name | X₄-X_{4′} | (s⁻¹) | (s⁻¹) | (nM) | (nM) | (M⁻¹s⁻¹) | (M⁻¹s⁻¹) | n |
| FX ins[194]>[DFLAEGGGVR]-HPC4 | GGVR-IVGG | 1.2E−03 | 2.9E−04 | 645 | 323 | 2.2E+03 | 7.8E+02 | 3 |
| 21k-HEP-[N]-FX | GGVR-IVGG | 2.6E−03 | 1.0E−03 | 2154 | 1007 | 1.3E+03 | 3.2E+02 | 3 |
| ins[194]>[DFLAEGGGVR]-HPC4 | | | | | | | | |
| 40k-HEP-[N]-FX | GGVR-IVGG | 1.7E−03 | 3.7E−04 | 1315 | 630 | 1.4E+03 | 3.8E+02 | 3 |
| ins[194]>[DFLAEGGGVR]-HPC4 | | | | | | | | |
| 73k-HEP-[N]-FX | GGVR-IVGG | 9.5E−04 | 1.8E−04 | 510 | 250 | 2.0E+03 | 5.8E+02 | 3 |
| ins[194]>[DFLAEGGGVR]-HPC4 | | | | | | | | |

### Example 20

### Stimulation of Thrombin Generation in Severe Haemophilia a Patient Plasma

### Materials and Methods

The amount of thrombin generated in plasma was measured by Calibrated Automated Thrombography (Hemker et al., “Calibrated Automated Thrombin Generation Measurement in Clotting Plasma,” Pathophysiol Haemost Thromb. 33:4-15 (2003); Hemker et al., “Thrombin Generation in Plasma: Its Assessment via the Endogenous Thrombin Potential,” Thromb Haemost. 74:134-138 (1995)). In a 96-well plate, 72 μL of Factor VIII deficient plasma pool (<1% residual activity, platelet-poor) from severe haemophilia A patients lacking Factor VIII inhibitors (George King Bio-Medical, Overland Park, Kans.) was incubated with 8 μL of recombinant Factor X variant (or HEPES-BSA buffer or recombinant Factor FVIII) for 10 minutes at 37° C. Reactions were started by adding 20 μL Thrombinoscope PPP LOW Trigger (1 μM tissue-factor and 4 μM phospholipid) and mixing with 20 μL fluorogenic substrate (Z-Gly-Gly-Arg-AMC) in HEPES-BSA buffer including 0.1 M CaCl₂. All reagents were pre-warmed to 37° C. The development of a fluorescent signal at 37° C. was monitored at 20 second intervals using a Fluoroskan Ascent reader (Thermo Labsystems OY, Helsinki, Finland). Fluorescent signals were corrected by the reference signal from the thrombin calibrator samples (Hemker et al., “Calibrated Automated Thrombin Generation Measurement in Clotting Plasma,” Pathophysiol Haemost Thromb. 33:4-15 (2003)) and actual thrombin generation in nM was calculated as previously described (Hemker et al., “Thrombin Generation in Plasma: Its Assessment via the Endogenous Thrombin Potential,” Thromb Haemost. 74:134-138 (1995)). Thrombin generation parameters peak thrombin, velocity index and endogenous thrombin potential (ETP) were calculated as previously described (Hemker et al., “Data management in thrombin generation,” Thromb Res 131:3-11 (2013)).

### Results

Table 15 below sets forth the thrombin generation parameters peak thrombin, velocity index and endogenous thrombin potential determined in haemophilia A plasma in the presence of HPC4-tagged thrombin sensitive FX molecules as well as the number of determinations (n).

Several FX molecules were able to stimulate thrombin generation in haemophilia A plasma compared to buffer. The observed peak thrombin concentration in haemophilia A plasma in the presence of HPC4-tagged thrombin sensitive FX molecules ranged from 6 nM to 275 nM with buffer and 100% Factor VIII (1 IU/mL) yielding peak thrombin concentrations of 18 and 112 nM, respectively.

TABLE 15
Thrombin Generation Parameters Determined in Haemophilia A Plasma in
the Presence of 300 nM of HPC4-tagged Thrombin Sensitive FX Molecules
(or HEPES-BSA buffer or 1 IU/mL of Factor VIII)

| | | Peak | Velocity | | |
|---|---|---|---|---|---|
| | | Thrombin | Index | ETP | |
| Compound Name | X₄-X_{4′} | (nM¹) | (nM × min⁻¹) | (nM × min) | n |
| HEPES-BSA Buffer | | 18.3 | 1.1 | 489 | 1 |
| Factor VIII (1 IU/mL) | | 112 | 21 | 1248 | 1 |
| FX [191-194][NATLSPR]-HPC4 | LSPR- | 5.8 | 0.2 | 268 | 1 |
| | IVGG | | | | |
| FX [191-194] > [NATLKPR]-HPC4 | LKPR- | 12.6 | 0.6 | 402 | 1 |
| | IVGG | | | | |
| FX [191-194] > [NATMQPR]-HPC4 | MQPR- | 15.8 | 0.9 | 397 | 1 |
| | IVGG | | | | |
| FX [191-194] > [NATLQPR]-HPC4 | LQPR- | 16.3 | 0.9 | 411 | 1 |
| | IVGG | | | | |
| FX [191-194] > [NATFKPR]-HPC4 | FKPR- | 16.9 | 0.8 | 489 | 1 |
| | IVGG | | | | |
| FX [191-194] > [NATLDPR]-HPC4 | LDPR- | 18.4 | 1.1 | 493 | 1 |
| | IVGG | | | | |
| FX [191-194] > [NATWQPR]-HPC4 | WQPR- | 21.8 | 1.3 | 521 | 1 |
| | IVGG | | | | |
| FX [191-194] > [MTPR]-HPC4 | MTPR- | 33.6 | 2.5 | 626 | 1 |
| | IVGG | | | | |
| FX-HPC4 | NLTR- | 46.1 | 5.8 | 904 | 1 |
| | IVGG | | | | |
| FX ins[194] > [STPSILLKPR]-HPC4 | LKPR- | 49.9 | 3 | 1061 | 1 |
| | IVGG | | | | |
| FX [191-194] > [NATLTPR]-HPC4 | LTPR- | 53.5 | 4 | 1034 | 1 |
| | IVGG | | | | |
| FX [191- | LDPR- | 53.8 | 4.6 | 948 | 1 |
| 194] > [GGGSGGGKEEEDIEFEEF | IVGG | | | | |
| ESSPKPDGSGGGSGGGNATLDPR]- | | | | | |
| HPC4 | | | | | |
| FX ins[194] > [DFLAEGGGVR]- | GGVR- | 58.9 | 4.5 | 999 | 1 |
| HPC4 | IVGG | | | | |
| FX ins[194] > [PSILFTPR]-HPC4 | FTPR- | 70.0 | 5.8 | 1083 | 1 |
| | IVGG | | | | |
| FX [191-194] > [LTPR]-HPC4 | LTPR- | 71.7 | 5.9 | 1166 | 1 |
| | IVGG | | | | |
| FX ins[194] > [DFLAEGGGPR]- | GGPR- | 274.5 | 74.9 | 1566 | 1 |
| HPC4 | IVGG | | | | |

### Example 21

### Cloning and Expression of Thrombin Sensitive FX Molecules

### A. Cloning of Thrombin Sensitive FX Molecules

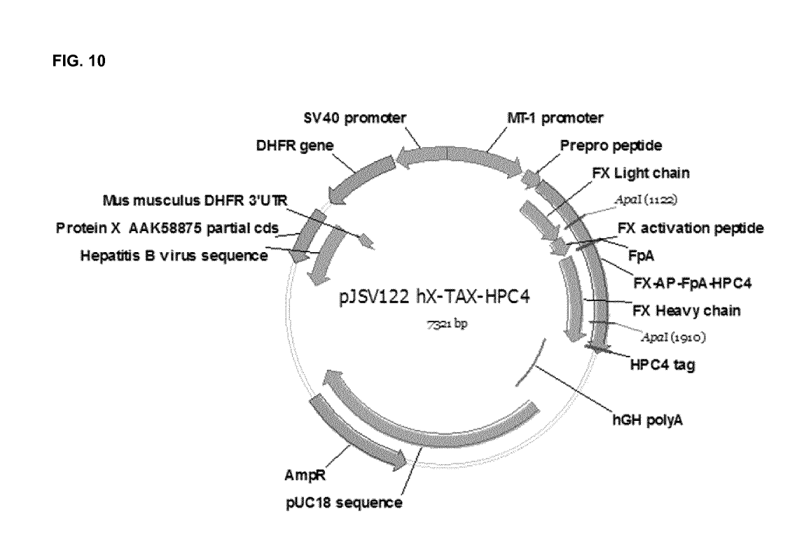

A thrombin sensitive Factor X construct (FX-FpA) cloned in the expression vector pNUT was received from INSERM (WO2004005347-A1 and Louvain-Quintard V B. et al. J Biol Chem. 2005 Dec. 16; 280(50):41352-9). The activation peptide from Factor X was inserted upstream from the FpA recognition sequence for thrombin to generate a construct encoding a protein identical to the protein described in EP2199387A1 as FX-AP-FpA (SEQ ID NO: 3). The generation of this construct was accomplished using the hereafter described cloning strategy. Using Factor X cDNA as a template, two partly overlapping PCR fragments were generated. The first PCR fragment contained a naturally occurring recognition site for the ApaI restriction enzyme located in the 3′ end of the light chain of Factor X, the sequence encoding the Factor X activation peptide and the inserted FpA sequence. The other fragment contained the sequence encoding the FpA sequence, the Factor X DNA sequence 3′ to the activation site of Factor X (Arg194-Ile195 in SEQ ID NO: 1) and included a naturally occurring recognition site for the ApaI restriction enzyme located in the heavy chain of Factor X. The two PCR fragments were mixed in a new PCR reaction to generate a DNA fragment containing the DNA sequence for the FX activation peptide fused to the FpA sequence and flanked by two ApaI restriction sites. Primers used for generating the two PCR fragments and for amplification of the fusion of the two fragments are shown in Table 16. The PCR fragment was cloned into the pNUT FX-FpA vector by digestion of both the PCR fragment and pNUT FX-FpA with ApaI and ligation of the two fragments using a Rapid DNA Ligation kit (Roche Applied Science, USA). A representation of the final construct is shown in FIG. 10.

TABLE 16
Primers Used for Generating the Two PCR Fragments
and for Amplification of the Fusion of the Two
Fragments Used in the Cloning of FX-AP-FpA

| | | SEQ | | SEQ |
|---|---|---|---|---|
| | | ID | | ID |
| | Primer S | NO | Primer AS | NO |
| PCR | GTACACCCTGGCTGA | 243 | GCATTCCTGGCCTC | 244 |
| fragment | CAACGGCAA | | CCACGATCCTCACG | |
| I | | | CCTCCTCCTTCAGCT | |
| | | | AGAAAGTCCCTGGT | |
| | | | GAGGTTGTTGTCGC | |
| PCR | GCGACAACAACCTCA | 245 | CGATGCCTGTCACG | 246 |
| fragment | CCAGGGACTTTCTAG | | AAGTAGGTGT | |
| II | CTGAAGGAGGAGGC | | | |
| | GTGAGGATCGTGGGA | | | |
| | GGCCAGGAATGC | | | |
| Fusion | GTACACCCTGGCTGA | 243 | CGATGCCTGTCACG | 246 |
| of PCR | CAACGGCAA | | AAGTAGGTGT | |
| fragments | | | | |

The full FX-AP-FpA-HPC4 cDNA and a desGla FX-AP-FpA-HPC4 cDNA were cloned into the pTT5 vector (Durocher Y. et al. Nucleic Acids Res. 2002 Jan. 15; 30(2):E9). The FX-AP-FpA CDS was sub-cloned into the pQMCF vector (Icosagen, Tartu, Estonia). Except for two sets of constructs (SEQ ID NOs: 229-236), all thrombin sensitive FX molecules were prepared by introduction of mutations into the FX-AP-FpA cDNA or derivatives of the FX-AP-FpA cDNA by either standard PCR-based site directed mutagenesis known in the art using the KOD Xtreme™ Hot Start DNA Polymerase (Novagen, Germany), followed by ligation of the DNA fragments using the In-Fusion HD Cloning Kit (Clontech, USA) or alternatively by using the QuickChange® Site-Directed Mutagenesis kit from (Stratagene, USA) by following the manufacturer's recommended instructions. For both methods primers were designed according to the manufacturer's recommendations. The two fragments that were not generated by these methods were generated by ordering of synthetic DNA sequences from Geneart (Life Technologies, USA). The ordered DNA fragments comprised a BspEI and AgeI fragment of Factor X and the desired variations in the Factor X gene. The DNA fragments were cloned into a BspEI and AgeI digested pQMCF vector using a Rapid DNA Ligation kit (Roche Applied Science, USA). The resulting variants, irrespective of cloning strategy, were in all cases expressed using the mammalian expression vector pQMCF (Icosagen, Tartu, Estonia) as a construct backbone. Introduction of the desired mutations was verified by DNA sequencing (MWG Biotech, Germany).

### B. Transfection and Expression of Thrombin Sensitive FX Molecules

A total of 10⁷CHO EBNALT85 cells (Icosagen, Estonia) were transfected with 10 μg of DNA using electroporation in a Bio-Rad Genepulser XCell™ apparatus (BioRad, USA). The transfected cells were seeded in 20 mL of QMIX1 media (a 1:1 mix of CD-CHO (Life Technologies, USA) and 293 SFM II (Life Technologies, USA) with 6 mM Glutamax and 10 mL/L of 50× HT supplement (Life Technologies, USA)) containing 5 μg/mL K-vitamin in 125 mL shake flasks (Corning, USA) immediately after transfection. The cells were cultured at 37° C., 8% CO₂ and 125 rpm in a Kuhner Climo-Shaker ISF1-X (Adolf Kuhner A G, Switzerland). A total of 10 mL fresh media and Geneticin (Life technologies, USA) to a final concentration of 700 μg/mL were added to the cells on day one or two after transfection.

Transfected CHO EBNALT85 cells were subcultured in QMIX1 media containing 5 μg/mL K-vitamin and 700 μg/mL Geneticin, by splitting the cells to a cell density of 3×10⁵ c/mL every three or four days. The culture volume was gradually increased to 100-200 mL. When viability of the transfected cells reached >90%, production was initiated by adding fresh media to the cells to a final volume of 1 L and a final cell density of 3×10⁵ c/mL. Production was performed by culturing cells for 7 days in 3 L shake flasks (Corning, USA) at 37° C., 8% CO₂ and 90 rpm. On day 3 or 4 cells were fed with 20% of the initial volume with Feed B (Life Technologies, USA) containing 6 mM Glutamax (Life Technologies, USA). On day 7, the culture media was harvested by centrifugation at 4600 rpm for 20 minutes. The supernatant was subsequently sterile filtered through a 0.22 μm Corning bottle-top vacuum filter (Corning, USA).

For larger scale production runs on a 10 L scale, CHO EBNALT85 cells transfected with FX molecule DNA were cultured in a 20 L Sartorius cultivation bag with an initial working volume of 8.5 L. The culture medium used consists of a basal medium (QMIX1 media) supplemented with 6 mM glutamine, 10 mL/L of 50× HT supplement (Life Technologies, USA)), 5 mg/L Vitamin K1, 700 mg/L Geneticin, and a feed medium, being CHO CD Efficient Feed B with 6 mM L-glutamine. The feed was supplied as a single bolus. The chosen process type for the production of the variants was a one week fed-batch process. The cultivation conditions are as follows; agitation was at 25-30 rpm with a rocking angle of 7°. Aeration was set to 5% CO₂ in air to headspace, 0.5-1 L/min and a temperature of 36.5° C. A 3% solution of Antifoam C (Sigma) was added to control foaming. Expression proceeded on the following schedule; on day 0 the seed culture was inoculated in basal medium to reach a target VCD of 0.3×10⁶ c/mL, on day 4 the feed solution was added (20% of initial volume) and on day 7 the culture was stopped and advanced to clarification. Off-line analysis of the cultures (days 0, (2), 4, (6), 7) included the following analytical assays: cell count and viability (Cedex HiRes), key metabolites (Nova Bioprofile), pH, pO₂, and pCO₂ (Siemens RapidLab/RapidPoint). Sampling for final product analysis (days 6 and 7) were taken as 2×200 μL cell-free supernatant in Micronic tubes (stored at −20° C.) and 1×1000 μL cell-free supernatant in glass HPLC vials with screw caps (stored at −20° C.). For clarification of harvest media, the harvest was filtered into sterile bags using a consecutive filter train consisting of disposable capsule filters; 3 μm Clarigard, Opticap XL10 (Millipore, USA) and 0.22 μm Durapore, Opticap XL10 (Millipore, USA). The clarified harvest was stored at 4° C. and delivered for immediate purification (or alternatively stored frozen for long term storage).

### Example 22

### Purification of Thrombin Sensitive Factor X Molecules

Typically 10 mM EDTA and 5 mM Benzamidine was added to the FX molecule harvests before being stored <72 h at +4° C. or >72 h but <14 days at −80° C. The purification was made with in-line dilution of the harvest with typically 30% Buffer A (30 mM HEPES pH 8.3, 10 mM EDTA and 5 mM Benzamidine) resulting in the starting sample having approximately a pH of 7.5 and having a conductivity of circa 10 mS/cm.

The first chromatography column was a Poros 50HQ AIEX column (GE Healthcare) equilibrated with 5 CV Buffer B (20 mM HEPES pH 7.5, 2 mM CaCl₂ and 5 mM Benzamidine). After applying the diluted harvest it was washed with 5 CV Buffer B and eluted with a step gradient to 100% elution buffer using 7 CV Buffer C (20 mM HEPES pH 7.5, 10 mM CaCl₂, 300 mM NaCl and 5 mM Benzamidine). The whole elution peak was collected and processed further.

The second chromatography step was an anti-HPC4 affinity column making use of the anti-HPC4 affinity towards the C-terminal HPC4 tag on the FX molecule. The anti-HPC4 antibody was covalently coupled to an epoxy-activated Sepharose 6B matrix (GE Healthcare) using a standard immobilisation technique. The affinity column was equilibrated with 5 CV of Buffer D (20 mM HEPES pH 7.5, 1 mM CaCl₂, 100 mM NaCl, 0.005% Tween 80 and 5 mM Benzamidine) and then the collected pool was directly loaded onto the column. The column was then washed through with 3 CV Buffer D, 4 CV Buffer E (20 mM HEPES pH 7.5, 1 mM CaCl₂, 1 M NaCl, 0.005% Tween 80 and 5 mM Benzamidine) and 3 CV Buffer D. The protein was eluted employing Buffer F (20 mM HEPES pH 7.5, 5 mM EDTA, 15 mM NaCl, 0.005% Tween 80 and 5 mM Benzamidine) and the entire elution peak was collected.

The third chromatography column was a small Poros 50HQ AIEX column (GE Healthcare), typically 5% of the CV of the previous affinity column. The Poros 50HQ AIEX column was equilibrated with Buffer B and after applying the sample, subsequently washed with Buffer B. Factor X molecules were then eluted with a step gradient employing Buffer C. The whole elution peak was collected and processed further.

As a last step an exchange of buffer using a PD-10 Desalting Column was done. The protein was applied and buffer exchanged according to the suppliers (GE Healthcare) instructions using Buffer G (10 mM MES pH 6.5, 1 mM CaCl₂ and 100 mM NaCl). The protein was then stored at −80° C.

### Example 23

### Oligonucleotide Primers Used in the Generation of Thrombin Sensitive Factor X Molecules

Table 17 below sets forth the oligonucleotide primers used for Factor X mutagenesis. The primer names correspond to the mutation, designated by the nomenclature outlined in Example 1 above, produced as a result of the mutagenesis using the primer. Primers are designated in the 5′ to 3′ direction and as either forward (-For) or reverse (-Rev) primer sets.

TABLE 17
Olionucleotide Primers for Used for Generation of Thrombin
Sensitive Factor X Molecules

| Primer Name | Primer Sequence (5′ to 3′) |
|---|---|
| desGla-FX ins[194] > [ATNATLDPR]- | GGTGAGGTTGTTGTCGCCCCTCTC |
| HPC4-For | AGGCTGCGTCTGGTTG |
| desGla-FX ins[194] > [ATNATLDPR]- | GGGGCGACAACAACCTCACCAGGA |
| HPC4 -Rev | AGGCCACCAATGCCACCCTGGATC |
| | CCAGAATCGTGGGAGGCCAGG |
| desGla-FX | GGTGAGGTTGTTGTCGCCCCTCTC |
| ins[194] > [DFLAEGGGPR]-HPC4 - | AGGCTGCGTCTGGTTG |
| For | |
| desGla-FX | GGGGCGACAACAACCTCACCAGGG |
| ins[194] > [DFLAEGGGPR]-HPC4 - | ATTTCCTGGCCGAGGGCGGCGGCC |
| Rev | CCAGAATCGTGGGAGGCCAGG |
| desGla-FX | GGTGAGGTTGTTGTCGCCCCTCTC |
| ins[194] > [DFLAEGGGVR]-HPC4 - | AGGCTGCGTCTGGTTG |
| For | |
| desGla-FX | GGGGCGACAACAACCTCACCAGGG |
| ins[194] > [DFLAEGGGVR]-HPC4 - | ATTTCCTGGCCGAGGGCGGCGGCC |
| Rev | CCAGAATCGTGGGAGGCCAGG |
| desGla-FX | GGTGAGGTTGTTGTCGCCCCTCTC |
| ins[194] > [DNEEGFFSAR]-HPC4 - | AGGCTGCGTCTGGTTG |
| For | |
| desGla-FX | GGGGCGACAACAACCTCACCAGGG |
| ins[194] > [DNEEGFFSAR]-HPC4 - | ATAATGAGGAGGGCTTCTTCAGCG |
| Rev | CCAGAATCGTGGGAGGCCAGGAAT |
| | GC |
| desGla-FX | GGTGAGGTTGTTGTCGCCCCTCTC |
| ins[194] > [DNSPSFIQIR]-HPC4-For | AGGCTGCGTCTGGTTG |
| desGla-FX | GGGGCGACAACAACCTCACCAGGG |
| ins[194] > [DNSPSFIQIR]-HPC4 - | ATAATAGCCCCAGCTTCATCCAGAT |
| Rev | CAGAATCGTGGGAGGCCAGGAATG |
| | CAAG |
| desGla-FX | GGTGAGGTTGTTGTCGCCCCTCTC |
| ins[194] > [HTHHAPLSPR]-HPC4 - | AGGCTGCGTCTGGTTG |
| For | |
| desGla-FX | GGGGCGACAACAACCTCACCAGGC |
| ins[194] > [HTHHAPLSPR]-HPC4 - | ACACCCACCACGCCCCCCTGAGCC |
| Rev | CCAGAATCGTGGGAGGCCAGG |
| desGla-FX | GGTGAGGTTGTTGTCGCCCCTCTC |
| ins[194] > [LSKNNAIEPR]-HPC4 - | AGGCTGCGTCTGGTTG |
| For | |
| desGla-FX | GGGGCGACAACAACCTCACCAGGC |
| ins[194] > [LSKNNAIEPR]-HPC4 - | TGAGCAAGAATAATGCCATCGAGC |
| Rev | CCAGAATCGTGGGAGGCCAGGAAT |
| | GCA |
| desGla-FX | CAACAACCTCACCAGGAACGAGTC |
| ins[194] > [NESTTKIKPR]-HPC4 -For | CACCACCAAGATCAAGCCCAGAAT |
| | CGTGGGAGGCCAGG |
| desGla-FX | CCTGGCCTCCCACGATTCTGGGCT |
| ins[194] > [NESTTKIKPR]-HPC4 - | TGATCTTGGTGGTGGACTCGTTCCT |
| Rev | GGTGAGGTTGTTG |
| desGla-FX | GGTGAGGTTGTTGTCGCCCCTCTC |
| ins[194] > [NRLAAALGIR]-HPC4 - | AGGCTGCGTCTGGTTG |
| For | |
| desGla-FX | GGGGCGACAACAACCTCACCAGGA |
| ins[194] > [NRLAAALGIR]-HPC4 - | ATAGACTGGCCGCCGCCCTGGGCA |
| Rev | TCAGAATCGTGGGAGGCCAGG |
| desGla-FX ins[194] > [PDNIAWYLR]- | GGTGAGGTTGTTGTCGCCCCTCTC |
| HPC4 -For | AGGCTGCGTCTGGTTG |
| desGla-FX ins[194] > [PDNIAWYLR]- | GGGGCGACAACAACCTCACCAGGC |
| HPC4 -Rev | CCGATAATATCGCCGCCTGGTACC |
| | TGAGAATCGTGGGAGGCCAGGAAT |
| | G |
| desGla-FX | CCAGACCTTCTTCAACCCCAGAATC |
| ins[194] > [SEYQTFFNPR]-HPC4 - | GTGGGAGGCCAGGAATGC |
| For | |
| desGla-FX | GTTGAAGAAGGTCTGGTACTCGCT |
| ins[194] > [SEYQTFFNPR]-HPC4 - | CCTGGTGAGGTTGTTGTCGCCC |
| Rev | |
| desGla-FX | GGTGAGGTTGTTGTCGCCCCTCTC |
| ins[194] > [STPSILPAPR]-HPC4 -For | AGGCTGCGTCTGGTTG |
| desGla-FX | GGGGCGACAACAACCTCACCAGGA |
| ins[194] > [STPSILPAPR]-HPC4 - | GCACCCCCAGCATCCTGCCCGCCC |
| Rev | CCAGAATCGTGGGAGGCCAGG |
| desGla-FX | GGTGAGGTTGTTGTCGCCCCTCTC |
| ins[194] > [TVELQGVVPR]-HPC4 - | AGGCTGCGTCTGGTTG |
| For | |
| desGla-FX | GGGGCGACAACAACCTCACCAGGA |
| ins[194] > [TVELQGVVPR]-HPC4 - | CCGTGGAGCTGCAGGGCGTGGTG |
| Rev | CCCAGAATCGTGGGAGGCCAGG |
| desGla-FX | |
| ins[194] > [YDEDENQSPR]-HPC4 - | GGTGAGGTTGTTGTCGCCCCTCTC |
| For | AGGCTGCGTCTGGTTG |
| desGla-FX | GGGGCGACAACAACCTCACCAGGT |
| ins[194] > [YDEDENQSPR]-HPC4 - | ACGATGAGGATGAGAATCAGAGCC |
| Rev | CCAGAATCGTGGGAGGCCAGGAAT |
| | GCA |
| FX [191-194] > [FTPR]-HPC4 -For | GACAACTTCACCCCCAGGATC |
| FX [191-194] > [FTPR]-HPC4 -Rev | CTGGGGGTGAAGTTGTCGCC |
| FX [191-194] > [ITPR]-HPC4 -For | AACATCACCCCCAGGATCGTGGGA |
| | GGCCAGGAA |
| FX [191-194] > [ITPR]-HPC4 -Rev | CCTGGGGGTGATGTTGTCGCCCCT |
| | CTCAGGCTGC |
| FX [191-194] > [LTPR]-HPC4 -For | GAGGGGCGACAACCTGACCCCCAG |
| | GATCG |
| FX [191-194] > [LTPR]-HPC4 -Rev | CGATCCTGGGGGTCAGGTTGTCGC |
| | CCCTC |
| FX [191-194] > [MTPR]-HPC4 -For | AACATCACCCCCAGGATCGTGGGA |
| | GGCCAGGAA |
| FX [191-194] > [MTPR]-HPC4 -Rev | CCTGGGGGTCATGTTGTCGCCCCT |
| | CTCAGGCTGC |
| FX [191-194] > [NATDTPR]-HPC4 - | CCACCGACACCCCCAGGATCGTGG |
| For | GAGGCCAGGAATGC |
| FX [191-194] > [NATDTPR]-HPC4 - | CTGGGGGTGTCGGTGGCGTTGTTG |
| Rev | TCGCCCCTCTCAGG |
| FX [191-194] > [NATFKPR]-HPC4 - | ACAACGCCACCTTCAAGCCCAGGA |
| For | T |
| FX [191-194] > [NATFKPR]-HPC4 - | ATCCTGGGCTTGAAGGTGGCGTTG |
| Rev | T |
| FX [191-194] > [NATFRPR]-HPC4 - | GCCACCTTCAGGCCCAGGATCGTG |
| For | GGAGGCCA |
| FX [191-194] > [NATFRPR]-HPC4 - | CCTGGGCCTGAAGGTGGCGTTGTT |
| Rev | GTCGCCCC |
| FX [191-194] > [NATFTPR]-HPC4 - | ACAACGCCACCTTCACCCCCAGGA |
| For | T |
| FX [191-194] > [NATFTPR]-HPC4 - | ATCCTGGGGGTGAAGGTGGCGTTG |
| Rev | T |
| FX [191-194] > [NATIKPR]-HPC4 - | CCACCATCAAGCCCAGGATCGTGG |
| For | GAGGCCAGGAATGC |
| FX [191-194] > [NATIKPR]-HPC4 - | CTGGGCTTGATGGTGGCGTTGTTG |
| Rev | TCGCCCCTCTCAGG |
| FX [191-194] > [NATIMPR]-HPC4 - | GCCACCATCATGCCCAGGATCGTG |
| For | GGAGGCCA |
| FX [191-194] > [NATIMPR]-HPC4 - | CCTGGGCATGATGGTGGCGTTGTT |
| Rev | GTCGCCCC |
| FX [191-194] > [NATIQPR]-HPC4 - | GCCACCATCCAGCCCAGGATCGTG |
| For | GGAGGCCA |
| FX [191-194] > [NATIQPR]-HPC4 - | CCTGGGCTGGATGGTGGCGTTGTT |
| Rev | GTCGCCCC |
| FX [191-194] > [NATIRPR]-HPC4 - | GCCACCATCAGGCCCAGGATCGTG |
| For | GGAGGCCA |
| FX [191-194] > [NATIRPR]-HPC4 - | CCTGGGCCTGATGGTGGCGTTGTT |
| Rev | GTCGCCCC |
| FX [191-194] > [NATITPR]-HPC4 - | GCCACCATCACCCCCAGGATCGTG |
| For | GGAGGCCA |
| FX [191-194] > [NATITPR]-HPC4 - | CCTGGGGGTGATGGTGGCGTTGTT |
| Rev | GTCGCCCC |
| FX [191-194] > [NATLDPR]-HPC4 - | CCACCCTGGACCCCAGGATCGTGG |
| For | GAGGCCAGGAATGC |
| FX [191-194] > [NATLDPR]-HPC4 - | CTGGGGTCCAGGGTGGCGTTGTTG |
| Rev | TCGCCCCTCTCAGG |
| FX [191-194] > [NATLEPR]-HPC4 - | CCACCCTGGAGCCCAGGATCGTGG |
| For | GAGGCCAGGAATGC |
| FX [191-194] > [NATLEPR]-HPC4 - | CTGGGCTCCAGGGTGGCGTTGTTG |
| Rev | TCGCCCCTCTCAGG |
| FX [191-194] > [NATLKPR]-HPC4 - | ACAACGCCACCCTGAAGCCCAGGA |
| For | T |
| FX [191-194] > [NATLKPR]-HPC4 - | ATCCTGGGCTTCAGGGTGGCGTTG |
| Rev | T |
| FX [191-194] > [NATLMPR]-HPC4 - | GCCACCCTGATGCCCAGGATCGTG |
| For | GGAGGCCA |
| FX [191-194] > [NATLMPR]-HPC4 - | CCTGGGCATCAGGGTGGCGTTGTT |
| Rev | GTCGCCCC |
| FX [191-194] > [NATLQPR]-HPC4 - | CCACCCTGCAGCCCAGGATCGTGG |
| For | GAGGCCAGGAATGC |
| FX [191-194] > [NATLQPR]-HPC4 - | CTGGGCTGCAGGGTGGCGTTGTTG |
| Rev | TCGCCCCTCTCAGG |
| FX [191-194] > [NATLRPR]-HPC4 - | GCCACCCTGAGGCCCAGGATCGTG |
| For | GGAGGCCA |
| FX [191-194] > [NATLRPR]-HPC4 - | CCTGGGCCTCAGGGTGGCGTTGTT |
| Rev | GTCGCCCC |
| FX [191-194] > [NATLSPR]-HPC4 - | GCCACCCTGTCCCCCAGGATCGTG |
| For | GGAGGCCA |
| FX [191-194] > [NATLSPR]-HPC4 - | CCTGGGGGACAGGGTGGCGTTGTT |
| Rev | GTCGCCCC |
| FX [191-194] > [NATLTPR]-HPC4 - | CCACCCTGACCCCCAGGATCGTGG |
| For | GAGGCCAGGAATGC |
| FX [191-194] > [NATLTPR]-HPC4 - | CTGGGGGTCAGGGTGGCGTTGTTG |
| Rev | TCGCCCCTCTCAGG |
| FX [191-194] > [NATMKPR]-HPC4 - | ACAACGCCACCATGAAGCCCAGGA |
| For | T |
| FX [191-194] > [NATMKPR]-HPC4 - | ATCCTGGGCTTCATGGTGGCGTTG |
| Rev | T |
| FX [191-194] > [NATMMPR]-HPC4 - | GCCACCATGATGCCCAGGATCGTG |
| For | GGAGGCCA |
| FX [191-194] > [NATMMPR]-HPC4 - | CCTGGGCATCATGGTGGCGTTGTT |
| Rev | GTCGCCCC |
| FX [191-194] > [NATMQPR]-HPC4 - | CCACCATGCAGCCCAGGATCGTGG |
| For | GAGGCCAGGAATGC |
| FX [191-194] > [NATMQPR]-HPC4 - | CTGGGCTGCATGGTGGCGTTGTTG |
| Rev | TCGCCCCTCTCAGG |
| FX [191-194] > [NATMRPR]-HPC4 - | GCCACCATGAGGCCCAGGATCGTG |
| For | GGAGGCCA |
| FX [191-194] > [NATMRPR]-HPC4 - | CCTGGGCCTCATGGTGGCGTTGTT |
| Rev | GTCGCCCC |
| FX [191-194] > [NATMTPR]-HPC4 - | GCCACCATGACCCCCAGGATCGTG |
| For | GGAGGCCA |
| FX [191-194] > [NATMTPR]-HPC4 - | CCTGGGGGTCATGGTGGCGTTGTT |
| Rev | GTCGCCCC |
| FX [191-194] > [NATWQPR]-HPC4 - | ACAACGCCACCTGGCAGCCCAGGA |
| For | T |
| FX [191-194] > [NATWQPR]-HPC4 - | ATCCTGGGCTGCCAGGTGGCGTTG |
| Rev | T |
| FX ins[194] > [DFLAEGGGPR]- | GACCAGTTCTGCCACGAGGAAC |
| HPC4 -For | |
| FX ins[194] > [DFLAEGGGPR]- | GGCCTCCCACGATCCTGGGGCCTC |
| HPC4 -Rev | CTCC |
| FX ins[194] > [PSILFKPR]-HPC4 - | ACCAGTTCTGCCACGAGGAAC |
| For | |
| FX ins[194] > [PSILFKPR]-HPC4 - | TCCCACGATTCTGGGCTTGAACAG |
| Rev | GATAGAGGGTCTGGTC |
| FX ins[194] > [PSILFTPR]-HPC4 - | ACCAGTTCTGCCACGAGGAAC |
| For | |
| FX ins[194] > [PSILFTPR]-HPC4 - | TCCCACGATTCTGGGGGTGAACAG |
| Rev | GATAGAGGGTCTGGTC |
| FX ins[194] > [PSILLKPR]-HPC4 - | CCCTCTATCCTGCTGAAGCCCAGA |
| For | ATCGTGGGAGGCCAGGAATGCAAG |
| | G |
| FX ins[194] > [PSILLKPR]-HPC4 - | CTTCAGCAGGATAGAGGGTCTGGT |
| Rev | CAGGTTGTTGTCGCCCCTC |
| FX ins[194] > [PSILLRPR]-HPC4 - | ACCAGTTCTGCCACGAGGAAC |
| For | |
| FX ins[194] > [PSILLRPR]-HPC4 - | TCCCACGATTCTGGGCCTCAGCAG |
| Rev | GATAGAGGGTCTGGTC |
| FX ins[194] > [PSILMKPR]-HPC4 - | ACCAGTTCTGCCACGAGGAAC |
| For | |
| FX ins[194] > [PSILMKPR]-HPC4 - | TCCCACGATTCTGGGCTTCATCAG |
| Rev | GATAGAGGGTCTGGTC |
| FX ins[194] > [PSILMTPR]-HPC4 - | ACCAGTTCTGCCACGAGGAAC |
| For | |
| FX ins[194] > [PSILMTPR]-HPC4 - | TCCCACGATTCTGGGGGTCATCAG |
| Rev | GATAGAGGGTCTGGTC |
| FX ins[194] > [PSILWQPR]-HPC4 - | ACCAGTTCTGCCACGAGGAAC |
| For | |
| FX ins[194] > [PSILWQPR]-HPC4 - | TCCCACGATTCTGGGCTGCCACAG |
| Rev | GATAGAGGGTCTGGTC |
| FX ins[194] > [STPSILFKPR]-HPC4 - | ACCAGTTCTGCCACGAGGAAC |
| For | |
| FX ins[194] > [STPSILFKPR]-HPC4 - | TCCCACGATTCTGGGCTTGAACAG |
| Rev | GATAGAGGGGGTAG |
| FX ins[194] > [STPSILFTPR]-HPC4 - | ACCAGTTCTGCCACGAGGAAC |
| For | |
| FX ins[194] > [STPSILFTPR]-HPC4 - | TCCCACGATTCTGGGGGTGAACAG |
| Rev | GATAGAGGGGGTAG |
| FX ins[194] > [STPSILLKPR]-HPC4 - | CCCTCTATCCTGCTGAAGCCCAGA |
| For | ATCGTGGGAGGCCAGGAATGCAAG |
| | G |
| FX ins[194] > [STPSILLKPR]-HPC4 - | CTTCAGCAGGATAGAGGGGGTAGA |
| Rev | TCTGGTCAGGTTGTTGTCGCCCCT |
| | C |
| FX ins[194] > [STPSILLRPR]-HPC4 - | ACCAGTTCTGCCACGAGGAAC |
| For | |
| FX ins[194] > [STPSILLRPR]-HPC4 - | TCCCACGATTCTGGGCCTCAGCAG |
| Rev | GATAGAGGGGGTAG |
| FX ins[194] > [STPSILMKPR]-HPC4- | ACCAGTTCTGCCACGAGGAAC |
| For | |
| FX ins[194] > [STPSILMKPR]-HPC4- | TCCCACGATTCTGGGCTTCATCAG |
| Rev | GATAGAGGGGGTAG |
| FX ins[194] > [STPSILMTPR]-HPC4- | ACCAGTTCTGCCACGAGGAAC |
| For | |
| FX ins[194] > [STPSILMTPR]-HPC4- | TCCCACGATTCTGGGGGTCATCAG |
| Rev | GATAGAGGGGGTAG |
| FX ins[194] > [STPSILWQPR]-HPC4- | ACCAGTTCTGCCACGAGGAAC |
| For | |
| FX ins[194] > [STPSILWQPR]-HPC4- | TCCCACGATTCTGGGCTGCCACAG |
| Rev | GATAGAGGGGGTAG |
| FX-HPC4 -For | CTCACCAGGATCGTGGGAGGCCAG |
| | GAATGC |
| FX-HPC4 -Rev | TCCCACGATCCTGGTGAGGTTGTT |
| | GTCGCC |

### Example 24

### Efficacy of Human Thrombin Sensitive FX Molecules in an Acute Haemophilia a Bleeding Model

FVIII deficient, FVIII-KO mice, 12-16 weeks old, male and females are divided into 3 groups of 12 animals, one for the test molecule, one negative control, and one positive control. Extra groups can be added in order to test more than one test compound. In each group, eight animals are subjected to tail bleeding and 4 animals are used in parallel for ex vivo efficacy testing using ROTEM analysis.

The mice are anaesthetised with isoflurane and placed on a heating pad. The tails are placed in pre-heated saline at 37° C. for 5 min.

Human concept molecules (wherein the concept molecule is any thrombin sensitive Factor X molecule listed in SEQ ID NO: 3-236) or vehicle or positive control (recombinant FVIII) is dosed i.v. in a dose volume of 5 ml/kg. The dose of the concept molecule is sufficient (as determined by in vitro characterisation of the concept molecule) to normalize the bleeding phenotype. The dose of the positive control, 5 U/kg recombinant FVIII, is also sufficient to normalize the bleeding phenotype.

After dosing the tail is placed back in the pre-heated saline for 5 minutes. For animals undergoing tail bleeding, a template-guided transection of the tail vein is performed exactly at the point where the tail diameter is 2.7 mm. After transection the tail is resubmerged in the pre-heated saline. Blood is collected over 60 minutes, and the haemoglobin concentration in the container is measured by spectrophotometry at 550 nm in order determine total blood loss.

Parallel animals are used for blood sampling and subsequent analysis of their clotting parameters (ex vivo efficacy). A blood sample is taken from the pen-orbital plexus with 20 μL capillary tubes without additive. The blood sample is diluted 1:10 in 0.13M sodium citrate and carefully mixed and stored at room temperature for immediate thromboelastography by ROTEM. The blood sample is re-calcified by adding 7 μL CaCl₂ to a mini cuvette (StarTEM).

Data are physically recorded throughout the experiment, aggregated and analysed in order to demonstrate the efficacy of the test molecules at reducing blood loss (tail bleeding) and clotting time (thromboelastography).

### Example 25

### Dose response study of Thrombin Sensitive FX Molecules in an Acute Haemophilia A Bleeding Model

FVIII deficient, FVIII-KO mice, 12-16 weeks old, male and females are divided into groups of 8 animals. Three to five groups are treated with increasing doses of the test molecule, one with vehicle (negative control), and one with recombinant FVIII (positive control). Further groups for extra doses of the test molecule can be added to the study.

The mice are anaesthetised with isoflurane and placed on a heating pad. The tails are placed in pre-heated saline at 37° C. for 5 min. Human concept molecules (wherein the concept molecule is any thrombin sensitive Factor X molecule listed in SEQ ID NO: 3-236), vehicle, or recombinant FVIII is dosed i.v. in a dose volume of 5 ml/kg. The doses of the test molecule are selected based on vitro characterisation in such a manner that they cover the dose/response window. The dose of the positive control, 5 U/kg recombinant FVIII, is sufficient to normalize the bleeding phenotype.

After dosing the tail is placed back in the pre-heated saline, and 5 minutes later a template-guided transection of the tail vein is performed exactly at the point where the tail diameter is 2.7 mm. The tail is resubmerged in the pre-heated saline. Blood is collected over 60 min and the haemoglobin concentration in the container is measured by spectrophotometry at 550 nm in order to determine total blood loss.

Data are physically recorded throughout the experiment, aggregated and analysed in order to determine the dose/response profile of the test molecule.

### Example 26

### Efficacy of Thrombin Sensitive FX Molecules in an Acute Haemophilia A Bleeding Model

An experiment as described in Example 24 is conducted, including an extra group of mice for each thrombin sensitive molecule to be tested (wherein the test molecule is any thrombin sensitive Factor X molecule listed in SEQ ID NO: 3-236). Recombinant FVIII is included as a positive control, vehicle as a negative, and for reference, the best concept molecule selected from experiments described in Examples 24 and 25 shall be included.

The collected data are analysed in order to demonstrate, how enhancing the activation of a thrombin sensitive FX molecule by thrombin affects blood loss and clotting time.

### Example 27

### Duration of Action of the Selected Lead Molecule(s) in an Acute Haemophilia A Bleeding Model

An experiment similar to the experiment described in Example 24 is conducted, including 8 groups of mice dosed with a single dose of the selected lead molecule (wherein the lead molecule is any thrombin sensitive Factor X molecule listed in SEQ ID NO: 3-236). The dose is selected based on the experiments described in Examples 24-26. These groups are treated i.v. at 5 minutes or 1, 3, 5, 12, 24, 48 or 72 hours prior to tail vein transection (animals for tail bleeding) or blood sampling (animals for ex vivo analysis by ROTEM). The collected data are analysed to determine blood loss (from tail bleeding) or clotting time (from ROTEM) in order to characterize the duration of action of the selected lead molecule.

### Example 28

### Verification of Efficacy in an Acute Bleeding Model of Inhibitor Complicated Haemophilia A

Female New Zealand white rabbits weighing approximately 2-3 kg are divided into 3 groups of each 8 animals. Two groups are made transiently haemophilic by i.v. administration of a monoclonal anti-FVIII-antibody (FVIII 4F30), thus mimicking the absence of FVIII activity and the presence of neutralizing antibodies found in inhibitor patients. The last group is left normal for reference. After 10 minutes, the rabbits are dosed intravenously with the test molecule (wherein the test molecule is any thrombin sensitive Factor X molecule listed in SEQ ID NO: 3-236) or vehicle, followed by induction of cuticle bleeding and a 60-minutes observation period. Blood is collected over the 60 minutes and the haemoglobin concentration in the container is measured by spectrophotometry at 550 nm in order to determine the total blood loss. Data are physically recorded throughout the experiment, aggregated, and analysed in order to demonstrate the efficacy at reducing blood loss in the inhibitor complicated haemophilia model.

### Example 29

### Establishment of the Thrombin Sensitive FX Concept as a Means of Bleeding Prophylaxis in Haemophilia A

Tolerance to human Factor X is induced by in rats with haemophilia A (FVIII-KO) by neonatal exposure to the human protein. During adolescence (from approximately 12 weeks of age), where haemophilia A rats experience spontaneous and frequently recurring bleeds, the rats are treated in a long term regimen mimicking clinical prophylaxis. The effect is assessed by monitoring the frequency and severity of bleeds as well as the resolution of their clinical manifestation. Data are analysed in order to demonstrate the effect of the test molecule as a prophylactic therapy in comparison with historic data on FVIII-KO rats undergoing on-demand treatment and/or prophylactic treatment with FVIII.

As an alternative to inducing tolerance to human Factor X, a rat specific surrogate of the test molecule can be utilized.

### Example 30

### Establishment of the Thrombin Sensitive FX Concept as a Treatment Principle in an Additional Non-Rodent Species

In addition to the rabbit study described in Example 28, pharmacodynamic experiments are conducted in dogs with haemophilia, which have accurately predicted effects as well as dosing requirements for other haemophilia treatments. The test molecule(s) (wherein the test molecule is any thrombin sensitive Factor X molecule listed in SEQ ID NO: 3-236) are administered i.v. using a dose volume of maximally 5 ml/kg in dogs with haemophilia A, at least 6 months of age. The effect is assessed ex vivo using surrogate markers, e.g. thrombelastography (as previously described in Knudsen et al. (2011) Haemophilia, 17: 962-970), or in vivo, e.g. using a standardized bleeding challenge and monitoring total blood loss, or bleeding time. Finally, test molecules may be administered to treat spontaneously bleeding dogs. In this setting, effects are monitored by assessing the resolution of clinical manifestation in comparison with historical data from an established treatment principle.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A thrombin sensitive Factor X molecule comprising 2 to 10 amino acid modifications N-terminally of the “IVGG” motif (amino acids 195 to 198 in SEQ ID NO: 1) in wild type Factor X, said modifications being in any of the positions X₁₀ to X₁:
X₁₀, X₉, X₈, X₇, X₆, X₅, X₄, X₃, X₂, X₁, I, V, G, G
wherein X₁₀ to X₁ can be any naturally occurring amino acid.

2. The thrombin sensitive Factor X molecule according to claim 1, wherein
X₈ is N
X₇ is N
X₆ is A
X₅ is T
X₄ is selected from the group consisting of L, I, M, F, V, P and W
X₃ is selected from the group consisting of Q, M, R, T, W, K, I, and V
X₂ is P, and
X₁ is R.

3. The thrombin sensitive Factor X molecule according to claim 1, wherein
X₈ is R
X₇ is G
X₆ is D
X₅ is N
X₄ is selected from the group consisting of L, I, M, F, W, P and W
X₃ is selected from the group consisting of T and S
X₂ is P, and
X₁ is R.

4. The thrombin sensitive Factor X molecule according to claim 1, wherein
X₉ is A
X₈ is T
X₇ is N
X₆ is A
X₅ is T
X₄ is selected from the group consisting of F, L, M, W, A, I, V and P
X₃ is selected from the group consisting of T, K, Q, P, S, Y, R, A, V, W, I and H
X₂ is P, and
X₁ is R.

5. The thrombin sensitive Factor X molecule according to claim 4, wherein X₃ is selected from the list consisting of: T, K and Q.

6. The thrombin sensitive Factor X molecule according to claim 4, wherein X₄ is selected from the list consisting of: F, L and M.

7. The thrombin sensitive Factor X molecule according to claim 4, wherein X₃ is T and X₄ is F.

8. The thrombin sensitive Factor X molecule according to claim 4, wherein X₃ is T and X₄ is M.

9. The thrombin sensitive Factor X molecule according to claim 1, wherein
X₁₀ is P
X₉ is E
X₈ is R
X₇ is G
X₆ is D
X₅ is N
X₄ is selected from the group consisting of L, I, M, F, V, P and W
X₃ is selected from the group consisting of T and S
X₂ is P, and
X₁ is R.

10. The thrombin sensitive Factor X molecule according to claim 1, wherein
X₁₀ is P
X₉ is E
X₈ is R
X₇ is G
X₆ is D
X₅ is N
X₄ is L
X₃ is T
X₂ is P, and
X₁ is R.

11. The thrombin sensitive Factor X molecule according to claim 1, wherein
X₁₀ is P
X₉ is E
X₈ is R
X₇ is N
X₆ is A
X₅ is T
X₄ is L
X₃ is T
X₂ is P, and
X₁ is R.

12. The thrombin sensitive Factor X molecule according to claim 1, wherein
X₁₀ is P
X₉ is E
X₈ is R
X₇ is G
X₆ is D
X₅ is N
X₄ is F
X₃ is T
X₂ is P, and
X₁ is R.

13. The thrombin sensitive Factor X molecule according to claim 1, wherein
X₁₀ is P
X₉ is E
X₈ is R
X₇ is G
X₆ is D
X₅ is N
X₄ is M
X₃ is T
X₂ is P, and
X₁ is R.

14. The thrombin sensitive Factor X molecule according to claim 1, wherein
X₁₀ is S
X₉ is T
X₈ is P
X₇ is S
X₆ is I
X₅ is L
X₄ is L
X₃ is K
X₂ is P, and
X₁ is R.

15. The thrombin sensitive Factor X molecule according to claim 1, wherein
X₁₀ is T
X₉ is R
X₈ is P
X₇ is S
X₆ is I
X₅ is L
X₄ is F
X₃ is T
X₂ is P, and
X₁ is R.

16. The thrombin sensitive Factor X molecule according to claim 1, wherein
X₁₀ is D
X₉ is F
X₈ is L
X₇ is A
X₆ is E
X₅ is G
X₄ is G
X₃ is G
X₂ is P, and
X₁ is R.

17. The thrombin sensitive Factor X molecule according to claim 1, wherein
X₁₀ is N
X₉ is E
X₈ is S
X₇ is T
X₆ is T
X₅ is K
X₄ is I
X₃ is K
X₂ is P, and
X₁ is R.

18. A pharmaceutical formulation comprising the thrombin sensitive Factor X molecule according to claim 1 and optionally one or more pharmaceutically acceptable excipients.

19. (canceled)

20. A method for treating a subject suffering from hemophilia, said method comprising administering to said subject a therapeutically effective amount of the pharmaceutical formulation according to claim 18.
